(19) United States
(12) Patent Application Publication
(10) Pub. No.: US 2016/0051579 A1
(43) Pub. Date: Feb. 25, 2016

(54) NITRIC OXIDE TREATMENT OF BOVINE RESPIRATORY DISEASE COMPLEX AND OTHER RESPIRATORY CONDITIONS

(71) Applicants: Alex STENZLER, Long Beach, CA (US); Chrlstopher C. MILLER, (US); Gilly REGEV-SHOSHANI, Vancouver (CA)

(72) Inventors: Alex Stenzler, Long Beach, CA (US); Christopher C. Miller, North Vancouver (CA); Gilly Regev-Shoshani, Vancouver (CA)

(73) Assignee: Bovicor Pharmatech, Inc., Vancouver, BC (CA)

(21) Appl. No.: 14/779,541

(22) PCT Filed: Dec. 30, 2014
(86) PCT No.: PCT/US2014/072868

§ 371 Date: Sep. 23, 2015

Related U.S. Application Data
(60) Provisional application No. 61/953,045, filed on Mar. 14, 2014.
(60) Provisional application No. 62/059,801, filed on Oct. 3, 2014.

(51) Int. Cl. A61K 33/00; A61K 9/00

(52) U.S. Cl. CPC A61K 33/00; A61K 9/0043

(57) ABSTRACT

Methods, devices, and systems for treating a respiratory condition using gNO delivered from a nitric oxide releasing solution (NORS) are disclosed and described. In one embodiment, the respiratory condition may be bovine respiratory disease complex (BRDc).

### PRIORITY DATA

This application is a 371 U.S. Nationalization of International Patent Application Serial No. PCT/US2014/072868, filed Dec. 30, 2014, which claims the benefit of U.S. Provisional Patent Application Ser. Nos. 61/953,045 and 62/059,801, filed on Mar. 14, 2014 and Oct. 3, 2014 respectively, each of which is incorporated herein by reference.

### FIELD OF TECHNOLOGY

The present technology relates to compositions, dosage forms, devices, systems, and associated methods for treating various diseases, disorders, and conditions. Accordingly, invention embodiments involve the fields of chemistry, pharmaceutical sciences, veterinary sciences, medicine, and other health sciences.

### BACKGROUND

Undifferentiated fever, also known as Bovine Respiratory Disease Complex (BRDc), is one of the more significant challenges faced by cattle producers and feedlot managers. It is the most economically important disease of beef cattle, accounting for 75% of the morbidity and over 50% of the mortality in feedlot cattle. The impact of BRDc is often two fold, namely, direct increased health costs for treating the condition, and secondly, a reduced growth performance seen in affected animals. Several viruses and bacteria have been associated with BRDc. The main bacterial pathogen of BRDc is Mannheimia haemolytica, which produces a potent leukotoxin that is its principal virulence factor.

It is clear that in cattle, as in other mammalian species, an active viral infection dramatically increases susceptibility to contracting bacterial pneumonia. This has been demonstrated experimentally in cattle infected with any one of several bovine respiratory viruses such as bovine herpes virus 1 (BHV-1) or bovine respiratory syncytial virus (BRSV), which render cattle highly susceptible to a secondary bacterial infection when challenged with M. haemolytica. These observations suggest that viral infection impairs host defense mechanisms against M. haemolytica, or amplifies undesirable aspects of the host response to this bacterial pathogen. Interestingly, by themselves, these pathogens are rarely capable of causing diseases in healthy cattle. However, when these two pathogens are combined with a compromised immune system and environmental stresses, animals are then even more likely to develop BRDc. Stress on an animal further contributes to illness susceptibility. Because of these factors, calves are at their most vulnerable point upon arrival at the feedlot and more likely to develop BRDc or other respiratory issues.

Consequently, the current practice is to administer antibiotics to all calves upon arrival at the feedlot during processing as a prophylactic treatment to reduce the incidence of BRDc. However, this practice is becoming less desirable due to, in large part, the emergence of drug resistant microorganisms and consumer concerns about residual antibiotics in the final beef or dairy product.

### BRIEF DESCRIPTION OF THE DRAWINGS

Invention features and advantages will be apparent from the detailed description which follows, and are further enhanced in conjunction with the accompanying drawings, which together illustrate, by way of example, various invention embodiments; and, wherein:

FIG. 1 is a graph showing an effect of pH on gNO production in one invention embodiment;

FIG. 2 is a graph showing an effect of pH on peak gNO concentration in one invention embodiment;

FIG. 3 is a graph showing an effect of nitrite concentration on gNO production in one invention embodiment;

FIG. 4 depicts a Hathback and Chemiluminescence device;

FIG. 5 comprises FIGS. 5A-5B, which depict the amount of gNO detected at 3, 8, 15 minutes (A) as well as 3, 4 (A) and 24 hours (B), where the X scale is Time (minutes) from start of measuring point (showing pre-measuring amount as 0-0.1 ppm) and Y scale showing amount of gNO (measured in ppb);

FIG. 6 is a graph depicting the antibacterial efficacy of NORS against A. baumanii, methicillin-resistant S. aureus, and E. coli using NORS of varying nitrite concentrations (0.07-0.41%) at pH 3.7, with controls of—saline, nitrites only at 0.41% (pH 6) and saline with reduced pH to 3.7, and wherein error bars indicate standard deviation for three experiments with 3 repetitions each;

FIG. 7 depicts LA/BHI agar plates, plated with A. baumanii, methicillin-resistant S. aureus, and E. coli following 10 min exposure to NORS;

FIG. 8 is a graph depicting the antiviral efficacy of NORS against Influenza H1N1 using NORS of varying nitrite concentrations (0.007-0.14%) at pH 3.7; where controls were—saline, nitrites only at 0.14% (pH 6) and saline with reduced pH to 3.7; and where error bars indicate standard deviation for three experiments with 3 repetitions each;

FIG. 9 comprises FIGS. 9A-9B which show 2 photos of plaque assay plates for A. saline control and NORS at 0.07% strength, B—pH control and NORS at 0.14% strength;

FIG. 10 is a schematic diagram of an apparatus constructed to test the effect of headspace gases generated by NORS on the mycelial growth;

FIG. 11 is a graph depicting the antifungal efficacy of NORS against Trichophyton mentagrophytes (FIG. 11A) and Trichophyton rubrum (FIG. 11B) using NORS of varying nitrite concentrations (0.007-0.07%); where error bars indicate standard deviation for three experiments with 3 repetitions each, and where an * represents significant (P<0.05) difference from control;

FIG. 12 is comprised of FIGS. 12A-12B which show a chromatogram of headspace gases found after 30 minutes of NORS exposure, and where there is shown a chromatogram produced by a GC-MS demonstrating the constituents of the headspace gas following 30 min exposure of 0.14% NORS, wherein GC-MS method was calibrated to quantify NO, N₂O, and NO₂ levels, and wherein A—GC chromatogram for MW=30—molecules detected are labeled above each peak, and B—MS chromatogram with the molecular weight detected at 5.4 min;

FIG. 13 shows antifungal activity of the head space gases produced from NORS, with two line graphs demonstrating: 1) Viability count of T. mentagrophytes mycelia (left Y axis) and 2) Nitrite levels in the exposed fungi solution (right Y axis) measured by Griess Reagent™ and showing both after being exposed to gases generated from NORS into the headspace for 2, 4, 8, 16, and 24 hours, with mycelial viability count shown as squares while nitrite concentration is shown as triangles, and error bars indicate standard deviation from triplicates;

FIG. 14 is a graph showing incidence of BRDc after 7 and 14 days post arrival to feedlot in one example;

FIG. 15 is a graph showing: (a) Initial weight (on arrival to feedlot—grey) vs. final weight (on Slaughter day—white) and (b) Average Temp for Tx and Control groups on each Tx day (grey=control, white=Tx) in one example;

FIG. 16 is a graph showing (a) MetHg levels before Tx, 5 and 30 min after Tx for the control animals, (b) MetHg levels before Tx, 5 and 30 min after Tx for the NO treated animals, and (c) Average difference in MetHg values 5 and 30 min post treatment compare to the values measured before treatment in one example grey=control, white=NO treatment;

FIG. 17 is a graph showing exhaled NO measure by chemiluminesence in (a) control and (b) NO treatment groups in one example;

FIG. 18 is a graph showing (a) The difference in nitrite concentration in samples after treatment (concentration in the 5 or 30 min post Tx samples minus concentration in the pre Tx samples), and (b) The average change per group in cortisol levels, calculated as cortisol level 30 min post Tx minus level pre Tx in one example, where grey=control, white=NO treatment.

FIG. 19 is a graph showing behavior analysis comparing control (grey) and NO treatment (white) group in one example, where values represent average of all animals in the group (4 for control and 5 for treatment).

FIG. 20 is a graph showing the consolidation and pleuritis (Gross observation), Tracheal and Lung (microscopic) score for the total 4 animals in each group in one example. Black=control, chessboard=treat-ment, Stripes=5× dose treatment;

FIG. 21 is a graph showing nitrites measured in different organs in one example. Black=control, chessboard=treat-ment, Stripes=5× dose treatment;

FIG. 22 is a graph showing viability of serotype 1 (stripes) and 6 (squares) of M. haemolytica after treatment with 60 mM NORS for 0.5, 1, 2, and 5 min in one example, and a star represents complete kill;

FIG. 23 is a graph showing the viability of virus using NORS and different initial titers for control (triangle), 1 min treatment (square) and 10 min treatment (circle) in one example, where saline was used as control and (a) IBR, (b) BRSV, (c) PI3;

FIG. 24 is a graph showing baseline weight distribution and across pens in one example;

FIG. 25 is a graph showing baseline weight gain distributions across pens in one example;

FIG. 26 is a graph showing total weight gain of antibiotic cohort (grey) and NORS cohort (white) during first 35 and 150 days on feed in one example;

FIG. 27 is a graph showing BRDc positive incidences in one example. The percentage of sick animals per pen and in each cohort in total;

FIG. 28 is a graph showing the average sick animals per cohort measure by 4 different ways in one example with antibiotic (grey) and NORS (white) cohorts;

FIG. 29 is a graph showing Haptoglobin levels in blood samples taken on arrival, at pull day and at day 35 in one example;

FIG. 30 is a graph showing total mortality during the first 35 days and during day 35-150 in one example, with antibiotic (grey) and NORS (white) cohorts, and where the red line represents the number of animals that died from respiratory disease;

FIG. 31 comprises two photos depicting the administration of NORS as a mist to ferrets;

FIG. 32, comprising FIGS. 33A-33B, depicts the changes in temperature (FIG. 32A) and viral titer (FIG. 32B) 1-5 days post viral installation and treatment with either saline (control) or NORS (0.41%) to ferrets;

These figures are provided to illustrate various aspects certain invention embodiments and are not intended to be limiting in scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Such alterations or variations may become apparent after a review of the present application. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a subject” includes a plurality of subjects.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, the term “veterinary subject” refers to a non-human animal or individual that may benefit from the administration of NORS or gNO produced by NORS, while the term “subject” simply refers to an animal or individual. Some non-limiting examples of veterinary subjects can include a bovine, goat, swine, foul, canine, feline, horse, bison, alpaca, llama, sheep, and the like. In one embodiment, the veterinary subject can be a bovine. In another embodiment, the veterinary subject can be a chicken, rooster, duck, goose, pheasant, or other fowl. In another embodiment, the veterinary subject can be a pig or other swine. In another embodiment, the veterinary subject can be a dog. In another embodiment, the veterinary subject can be a cat. In a further embodiment, can be a ferret or a mink. In yet another embodiment, the veterinary subject can be a commercially salable animal.

As used herein, the terms “treat,” “treatment,” or “treating” when used in conjunction with the administration of NORS, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition. Further, these terms can encompass metaphylactic acts of administering NORS to bovine in anticipation of an expected outbreak of disease. Moreover, a “treatment outcome” refers to a result obtained at least in part, due to behavior or an act taken with regard to a subject. Treatment outcomes can be expected or unexpected. In one specific aspect, a treatment outcome can be a delay in occurrence or onset of a disease or conditions or the signs or symptoms thereof.

As used herein, the term “metaphylactic” refers to acts of mass medication of a group of subjects as a matter of policy or procedure to minimize or prevent the outbreak of a disease or disorder.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases nitric oxide.

As used herein “NORS” refers to a nitric oxide (NO) releasing solution, composition or substance. In one aspect, NO released from NORS may be a gas.

As used herein a “therapeutic agent” refers to an agent that can have a beneficial or positive effect on a subject when administered to the subject in an appropriate or effective amount. In one aspect, NO can be a therapeutic agent.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen,” or a “metaphylactic dosing regimen” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the exact numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Nitric Oxide (NO) is a free-radical which is lipophilic with a small stokes radius making it an excellent signaling molecule enabling it to readily cross the plasma membrane into the cytosol, and is therefore believed to be suitable for treatment of a variety of indications, including in veterinary subjects. For example, undifferentiated fever, also known as Bovine Respiratory Disease Complex (BRDc), is one of the more significant challenges faced by cattle producers and feedlot managers. It is the most economically important disease of beef cattle with at least a two-fold impact. The first impact is direct increased health costs for treating the condition. The second impact is reduced growth performance seen in affected animals. Several viruses and bacteria have been associated with BRDc, but the main bacterial pathogen of BRDc is Mannheimia haemolytica. It is clear that in cattle, as in other animal species, an active viral infection dramatically increases susceptibility to contracting bacterial pneumonia. This has been demonstrated experimentally in cattle infected with any one of several bovine respiratory viruses such as bovine herpes virus 1 (BHV-1) or bovine respiratory syncytial virus (BRSV), which render cattle highly susceptible to a secondary bacterial infection when challenged with M. haemolytica. These observations suggest that viral infection impairs host defense mechanisms against M. haemolytica, or amplifies undesirable aspects of the host response to this bacterial pathogen. By themselves, these pathogens are rarely capable of causing diseases in healthy cattle. However, when these two pathogens are combined with a compromised immune system and/or environmental stresses, animals are then even more likely to develop BRDc. Because of these factors, calves are at their most vulnerable point upon arrival at the feedlot and more likely to develop BRDc.

The current method of preventing BRDc in cattle is to administer metaphylactic antibiotics to all calves upon arrival at the feedlot. However, this practice presents major concerns, in large part, because of the emergence of drug resistant microorganisms and the residual antibiotics in the final product. Hence, there is a growing urgency to find more effective non-antibiotic based alternatives for metaphylactic treatment of cattle to curb BRDc incidence. BRDc is only one non-limiting example of a health condition that can cause great hardship upon livestock, pets, and other animal subjects. A variety of other non-limiting health conditions are also described herein.

Invention embodiments relate to formulations, devices, systems, and methods for treating a disease or disorder in a subject. Such embodiments can include administration of a liquid nitric oxide releasing solution (NORS) to a subject as a vehicle for releasing an effective amount of gaseous nitric oxide (gNO) to a site or situs of administration and/or to a targeted treatment site or situs that is distal to the administration site. Administration of the liquid NORS provides for the quick delivery of the liquid NORS to the targeted treatment site, followed by an extended and prolonged release of gNO at the treatment site or situs. However, gNO can be administered utilizing a variety of formulations.

The present technology provides a number of advantages over currently used NO treatments in veterinary and other applications. For example, as presented herein, it has been unexpectedly found that in one embodiment, the NORS of the present technology is capable of releasing a therapeutically effective amount of gNO for an extended period of time while using a lower amount of one or both of a nitrite component and an acidifying agent than previously thought. Also as presented herein, it has been unexpectedly found that when the compositions of the instant invention are formulated as a liquid rather than as a cream or lotion, a surprising and significantly more effective administration of gNO is achieved, including a longer duration of gNO release and therefore the ability to use of a reduced amount or dosage of the composition. Moreover, unlike topical applications that are applied directly to the lesion and therefore have an area of treatment limited to only the site of application, gNO released from a liquid NORS can also treat lesions or microbes that are not at the site of NORS application. For example, the liquid NORS can be sprayed into the nostrils of the subject, resulting in the extended release of gNO into the subject's aspirated air stream over minutes to hours. Furthermore, the duration of treatment can be reduced to a single treatment versus multiple treatments over weeks to months.

The NORS can provide an extended release of gNO to a subject in need thereof. By “extended release,” it is meant that an effective amount of gNO is released from the formulation at a controlled rate for a specified duration such that therapeutically beneficial levels (but below toxic levels) of the component are maintained over an extended period of time following NORS administration. Thus for example, release can occur about 5 seconds to about 24 hours, thus, providing, for example, a 30 to 60 minute, or several hour, dosage form. In one embodiment, the NO gas is released over a period of at least 30 minutes. In another embodiment, the NO gas is released over a period of at least 8 hours. In another embodiment, the NO gas is released over a period of at least 12 hours. In another embodiment, the NO gas is released over a period of at least 24 hours. An extended release NORS is beneficial in that the solution can be administered to the subject over a short period of time, while the release of NO from the solution continues following administration. Moreover, the use of an extended release NORS allows the subject to remain ambulatory following administration of the solution, as opposed to remaining stationary while being connected to a NO-releasing device in order to receive treatment.

In some regards, the release performance of a NORS can be quantified using a relationship between the amount of time required to administer the NORS and the amount of time over which NO is released from the NORS. For example, when NORS administration takes 5 seconds and NO release extends for 24 hours. In one embodiment, the ratio of NORS administration time to NO release time can be from about 1:1 to about 1:90,000. In another embodiment, the ratio can be at least about 1:10,000. In another embodiment, the ratio can be at least 1:1000. In a further embodiment, the ratio can be at least about 1:500. In yet another embodiment, the ratio can be at least about 1:100. In an additional embodiment, the ratio can be at least about 1:50.

In one aspect, the NORS can have antibacterial, antifungal, and/or antiviral properties, and therefore may be useful as antibacterial, antifungal, and/or antiviral agents. In one embodiment, the NORS can be an antibacterial agent effective against Acinetobacter baumanii. In another embodiment, the NORS can be an antibacterial agent effective against Methicillin-resistant Staphylococcus aureus. In another embodiment, the NORS can be an antibacterial effective against Escherichia coli. In one embodiment, the NORS can be an antifungal agent effective against Trichophyton rubrum. In another embodiment, the NORS can be an antiviral agent effective against Influenza, for example H1N1.

In one embodiment, a method is described for administering a NORS to a subject. Such a method can include providing a nitric oxide releasing compound or agent and an acidifying compound or agent. The nitric oxide releasing agent and the acidifying agent can be combined to provide an activated NORS. Any suitable nitric oxide releasing compounds and acidifying compounds can be used, as described herein. Strong acidifying agents can cause rapid production and release of gNO. Weaker acidifying agents can produce a prolonged production and release of gNO. Careful control of the amounts and combinations of acidifying agents incorporated in the NORS can prolong the release of a therapeutically effective amount of gNO, thus allowing the NORS to be prepared well in advance of administration. However, this is not always a desirable scenario. In some cases, it can be desired to produce a strong and sudden burst of gNO at the site of the disease, disorder, or condition being treated, and thus combining the nitric oxide releasing compound and the acidifying agent just prior to administration or during administration, or at the treatment site can be preferred. Alternatively, it may be desirable to administer either the acidifying agent or the nitric oxide releasing agent to the subject and subsequently activate it by administration of the corresponding nitric oxide releasing agent or acidifying agent. Hence, the NORS can be administered to a subject before, during, or after activation of the NORS. In one aspect, the NORS can be activated up to 24 hours before administration. In one aspect, the NORS can be activated up to 8 hours before administration. In one aspect, the NORS can be activated up to 1 hour before administration. In one aspect, the NORS can be activated up to 30 minutes before administration. In one aspect, the NORS can be activated up to 10 minutes before administration. In one aspect, the NORS can be activated up to 5 minutes before administration. In one aspect, the NORS can be activated up to 1 minute before administration. In another aspect, the NORS can be activated during administration. In another aspect, the NORS can be activated after administration.

The NORS may be administered to the subject as an extended release formulation of gNO, and optionally with a carrier formulation, such as microspheres, microcapsules, liposomes, etc. The NORS can be administered topically or internally, such as an intranasal injection, to treat a microbial (i.e. viral or bacterial) disease or disorder. Additionally, the NORS can be administered as a liquid, a spray, a vapor, micro-droplets, mist, footbath, or any other form that provides the desired release of gNO from the NORS.

The duration of administering the NORS to the subject may be varied in order to optimize delivery. In one embodiment, the NORS is administered to the subject over a time period of about 1 second or less. In another embodiment, administration time can be about 5 seconds or less. In another embodiment, administration time can be about 5 seconds. In another embodiment, the administration time can be about 30 seconds or less. In another embodiment, the administration time can be about 1 minute or less. In another embodiment, the administration time can be about 2 minutes or less. In another embodiment, the administration time can be about 10 minutes or less. In another embodiment, the administration time can be about 30 minutes or less.

One embodiment includes a method of treating a subject for a condition, disease, or disorder for which NO provides a therapeutic effect (i.e. which is responsive to NO therapy), by delivering a NORS to a treatment site of the subject to treat any disease, disorder, or condition. In some aspects, administration of NORS may be made to a site that is different from, including distal from, a target administration site, but which location still allows NO to reach the intended treatment or administration site and have a therapeutic effect. Such diseases can include, but are not limited to respiratory diseases, respiratory infections, sinus infections, throat infections, ear infections, eye infections, wounds, burns, topical infections, inflammatory diseases, and the like. In another aspect, the disease, disorder, or condition can include a surgical site. In one embodiment, NORS can be administered as part of routine preventative post-operative care.

In one embodiment, the method comprises the treatment of a respiratory disease or disorder in a subject. Exemplary respiratory diseases or disorders treated include bovine respiratory disease (BRDc), bovine herpes virus 1 (BVH-1), bovine respiratory syncytial virus (BRSV), porcine respiratory disease complex (PRDC), and the like. In another embodiment, the method can include treatment of and reduction of inflammation of the upper respiratory tract. In certain embodiments, the method comprises the treatment of a respiratory disease or disorder caused by a bacterial, fungal, or viral infection. In some embodiments, the infection is caused by a bacterium. In other embodiments, the infection is caused by a virus. Such treatment of a respiratory disease can include the delivery of a NORS into the upper respiratory tract of the subject to be treated. For example, in certain embodiments, the NORS may be injected, sprayed, inhaled, or instilled into the respiratory tract. The NORS may be administered to the respiratory tract via the nasal cavity or oral cavity. In one embodiment, the NORS is sprayed into the upper respiratory tract. In one embodiment, the NORS is administered intranasally. In one embodiment, the NORS is administered to the nasal passages or the sinuses or both. The NORS provides for extended gNO production, thereby providing continuous delivery of therapeutic gNO to the upper and/or lower respiratory tract.

In one embodiment, the method comprises the treatment of a respiratory disease or disorder, wherein the disease or disorder is caused by an infection. For example, the infection may be caused by a virus, a fungus, a protozoan, a parasite, an arthropod or a bacterium, including a bacterium that has developed resistance to one or more antibiotics. In some embodiments, the infection is caused by a bacterium. In other embodiments, the infection is caused by a virus.

One embodiment includes a method of administering a therapeutically effective amount of gaseous nitric oxide (gNO) to a subject, wherein occurrence of a respiratory condition is reduced. In one aspect, the method can be used to treat a condition that includes at least one of bovine respiratory disease (BRDc), bovine herpes virus 1 (BVH-1), bovine respiratory syncytial virus (BRSV), porcine respiratory disease complex (PRDC), and the like. In one specific aspect, the method can be used to treat BRDc. In one aspect, the method can be used on an individual basis as a prophylactic treatment of such conditions. In one aspect, the method can also be used with large groups of subjects as a metaphylactic treatment of such conditions.

In one embodiment, the method comprises the treatment of an infection in a subject, including infections caused by a virus, a fungus, a protozoan, a parasite, an arthropod or a bacterium, including a bacterium that has developed resistance to one or more antibiotics. In some embodiments, the infection is caused by a bacterium. In one embodiment, the infection is by a virus. In other embodiments, the infection is caused by a fungus.

In one embodiment, the NORS can include the use of water or a saline-based solution or substance and at least one NO releasing compound, such as nitrite or a salt thereof. In one embodiment, the NORS is a saline-based solution or substance. In one embodiment, the NO releasing compound is a nitrite, a salt thereof, or any combinations thereof. Non-limiting examples of nitrites include nitrite salts such as sodium nitrite, potassium nitrite, barium nitrite, and calcium nitrite, mixed salts of nitrite such as nitrite orotate, and nitrite esters such as amyl nitrite. In one embodiment, the NO releasing compound is selected from the group consisting of sodium nitrite and potassium nitrite, or any combinations thereof. In another embodiment, the NO releasing compound is sodium nitrite. In one embodiment, the NORS can comprise a sodium nitrite in a saline solution. In another embodiment, the solution can comprise a potassium nitrite in a saline solution.

In one embodiment, the concentration of NO releasing compound, for example, nitrite (i.e. NO₂), in the NORS can be from 0.07% w/v to about 1.0% w/v. In one embodiment, the concentration of nitrites in the solution is no greater than about 0.5% w/v. In another embodiment, the concentration of nitrites in the solution is about 0.1% w/v. In a further embodiment, the concentration of nitrites in the solution is about 0.2% w/v. In an additional embodiment, the nitrite concentration is about 0.3% w/v. In another embodiment, the nitrite concentration is about 0.4% w/v. In yet another embodiment, the concentration of nitrite in the solution is about 0.28% w/v. In an additional embodiment, the nitrite concentration in the solution is about 0.32% w/v. In an additional embodiment, the nitrite concentration in the solution is about 0.38% w/v. In another embodiment, the nitrite concentration in the solution is about 0.41% w/v. In a further embodiment, the nitrite concentration in the solution is about 0.46% w/v. In another embodiment, the nitrite concentration in the solution is from about 0.07% w/v to about 0.5% w/v. In a further embodiment, the nitrite concentration in the solution can be from about 0.05% w/v to about 10% w/v. As used herein, the term “w/v” refers to the (weight of solute in grams/100 mLs of volume of solution)×100%. In one embodiment, when sodium nitrite is used in the solution, the concentration of sodium nitrite can be from about 0.41% w/v to about 0.69% w/v. Other nitrite salts can be used as a source of NO₂ and the specific amount of each required to provide appropriate NO₂ concentrations and concentration ranges as herein described can be determined by one of ordinary skill in the art in view of the present disclosure.

In an additional embodiment, the amount of NO releasing agent, for example nitrite (i.e. NO₂), can be a concentration of from about 1 mM to about 1M. In another embodiment, the nitrite concentration can be from about 10 mM to about 500 mM. In yet a further embodiment, the nitrite concentration in the solution can be from about 100 mM to about 200 mM. In an additional embodiment, the nitrite concentration in the solution can be from about 40 mM to about 180 mM. In a further embodiment, the nitrite concentration in solution can be about 160 mM. In an additional embodiment, the nitrite concentration in solution can be from about 40 mM to about 120 mM. In another embodiment, the nitrite content can be from about 51 mM to about 100 mM. In another embodiment, the nitrite concentration can be about 60 mM. In yet another embodiment, the concentration can be 100 mM. In an additional embodiment the concentration of nitrite in the solution can be about 109 mM or less. In a further embodiment, when sodium nitrite is used in the solution, the concentration of sodium nitrite can be about 72 mM. Again, other nitrite salts can be used as a source of NO₂ and the specific amount of each required to provide appropriate NO₂ concentrations and concentration ranges as herein described can be determined by one of ordinary skill in the art in view of the present disclosure.

In one embodiment, the NORS can also contain at least one acidifying agent. As described elsewhere herein, the addition of at least one acidifying agent to the NORS solution contributes toward increased production (i.e. attenuates production) of NO from the NORS solution or substance. Any acidifying agent which contributes to NO production is contemplated by the present technology. In one embodiment, the acidifying agent can be an acid. In one aspect, the acid can be an organic acid. In another aspect, the acid can be an inorganic acid. Non-limiting examples of acids include ascorbic acid, salicylic acid, malic acid, lactic acid, citric acid, formic acid, benzoic acid, tartaric acid, carbonic acid, hydrochloric acid, sulfuric acid, nitric acid, nitrous acid and phosphoric acid. In one embodiment, the acid is selected from the group consisting of ascorbic acid, citric acid, malic acid, hydrochloric acid, and sulfuric acid, or any combinations thereof. In another embodiment, the acid is citric acid. Alternatively, the acidifying agent can include an acidifying gas such as NO, N₂O, NO₂, CO₂, and other acidifying gases. In one aspect, the acidifying gas may be NO. In another aspect, the acidifying agent can be an acidifying polymer or protein, such as alginic acid, an acidified gelatin, polyacrylic acid, and other acidifying polymers or proteins. In addition, acidifying agents may include compounds or molecules that produce or release an acid, including any of the aforementioned acids, upon addition to the NORS solution.

As described above, the amount of acidifying agent present in the solution can affect the rate of the reaction to produce NO. In one embodiment, the amount of acidifying agent is no greater than about 5.0% w/v of the solution. In another embodiment, the amount of acidifying agent is no greater than about 0.5% w/v. In another embodiment, the amount of acidifying agent is about 0.2% w/v. In a further embodiment, the amount of acidifying agent is about 0.07% w/v. In an additional embodiment, the amount of acidifying agent is about 0.07% w/v. In a further embodiment, the amount of acidifying agent is about 0.04% w/v. In yet another embodiment, the amount of acidifying agent is between about 0.07-5.0% w/v. In another embodiment, the amount of acidifying agent can be from about 2 mM to about 600 mM. In another embodiment, the amount of acidifying agent can be from about 5 mM to about 100 mM. In another embodiment, the amount of acidifying agent can be from about 5 mM to about 50 mM. In another embodiment, the amount of acidifying agent can be from about 100 mM to about 600 mM. It will be recognized that different acidifying agents can lower the NORS pH at different rates and to different degrees depending their specific properties and nature and suitable concentrations and concentration ranges of a given acidifying agent that are suitable for use as recited herein can be determined by one of ordinary skill in view of the present disclosure.

In one embodiment, a therapeutically effective amount of gNO can be administered to or within the nares or nasal cavity of a subject or to an area sufficiently proximate to the nares to facilitate inhalation of the gNO. Once the gNO is inhaled it can travel through the nasal passage and trachea into the lungs. In one aspect, gNO can be administered as a NORS. In another aspect, the NORS can be a solution. In another aspect the NORS can include a carrier or additive other than water. In a more specific aspect, the solution can be administered as at least one of a liquid and an aerosol. In some aspects, the NORS may enter the subject's sinus cavity. In some aspects, the NORS can be deposited beyond the nasal vestibule of the subject. In one aspect, from about 40% to about 100% of the NORS can be deposited beyond the nasal vestibule. In another aspect, from about 10% to about 80% of the NORS can be deposited beyond the nasal vestibule of the subject. In another aspect, from about 40% to about 60% of the NORS can be deposited beyond the nasal vestibule.

In one aspect, a therapeutically effective amount can be from about 40 to about 1000 ppm gNO. In one embodiment, the therapeutically effective concentration of gNO is from about 4 ppm to about 400 ppm gNO. In another aspect, the therapeutically effective amount of gNO can be from about 100 to about 220 ppm gNO. In another embodiment, the therapeutically effective concentration is from about 50 to about 200 ppm gNO. In a more specific aspect, the therapeutically effective amount can be about 160 ppm gNO. In another aspect, the therapeutically effective amount can be less than 160 ppm. A NORS that releases a therapeutically effective amount of gNO can be deposited in or on the subject's nose in an amount from about 1 to 100 nil. In another aspect, a NORS that releases a therapeutically effective amount of gNO can be deposited in or on the subject's nose in an amount from about 1 to about 50 ml. In a more specific aspect, a NORS that releases a therapeutically effective amount of gNO can be deposited in or on the subject's nose in an amount of about 32 nil. Such amounts can be made through a single administration or an administration event that includes multiple administrations.

Nearly any device capable of administering NORS to or within the nares or nasal cavity or to the tissue or areas surrounding the nares or nasal cavity can be used. In many cases, at least one of a syringe or spray device can be used. In another aspect, the NORS can be wiped or smeared into a portion of the nares or surrounding tissues or area. In another aspect, the muzzle of the subject can be immersed in the NORS for a brief period of time, such as from about 1 to about 5 seconds. Hence, the NORS can be administered to or deposited in or on the nose of the subject, or both. Further, the NORS can be administered to each nostril simultaneously or in succession. The NORS may be administered in a single dosage to each nostril or as two or more dosages in succession.

In some embodiments, the formulation may be an immediate release formulation. In other embodiments, the formulation can be a controlled release formulation which releases g NO for an extended period of time. In some embodiments, the carrier of the solution may be water, or a saline solution. In other embodiments, other carriers can be used.

A NORS can be prepared as a single phase or multi-phase formulation. In one embodiment the NORS can be formulated as a two-phase or two-part composition that includes at least one nitrite or salt thereof in a first part of the two-part composition, and an acidifying agent in the second part of the two-part composition. The acidifying agent can be a liquid, gas, or solid as previously mentioned.

When administering a NORS and/or a gNO to an internal tissue (or any in-vivo situs, location, or environment), in some embodiments, the NORS can be prepared as a liquid solution that can be delivered as a solution or as an aerosol. In some aspects, a liquid solution can minimize the stress caused to the subject upon administration of the NORS and throughout the treatment period.

In one embodiment, the NORS can be characterized as having multiple states of activity, such as a dormant state and an active state. Furthermore, the active state may have one or more sub-states where release rate or activity of the NORS varies. As contemplated herein, the dormant state of the NORS is one in which the pH of the solution or substance is above 5.0 and exhibits a minimal or undetectable production level of nitric oxide gas. In one embodiment, the pH of the dormant state of the NORS is between a pH of about 5.0 and a pH of about 7.0. The active state of the NORS is one in which the pH of the solution is below 5.0 and exhibits an increased or enhanced production level of nitric oxide gas, including production at a therapeutically effective level, and in some embodiments, over an extended period of time. In one embodiment, the pH of the active state of the NORS is between a pH of about 1.0 and a pH of about 5.0 and may include a number of sub-states characterized by amount or rate of gNO release. In another embodiment, the pH of the active state of the NORS is between a pH of about 3.0 and a pH of about 5.0. In one embodiment, the pH is about 3.2. In another embodiment, the pH is about 3.6. In yet another embodiment, the pH is about 3.5. In yet a further embodiment, the pH is about 3.4. In an additional embodiment, the pH is about 3.3. In yet another embodiment, the pH can be from about 3.0 to about 3.5. Because the NORS of the present invention can have multiple states of activity or inactivity, the solution or substance can be pre-made, transported and set up for administration while in its dormant state (pH greater than 5.0), without losing any appreciable amount of gNO or without losing its ability to produce a therapeutically effective amount of gNO. Once a user is ready to deliver or administer the solution or substance to a subject, the solution or substance can be activated prior to administration to the subjection, for example, immediately prior (pH driven below 5.0), thereby maximizing the amount of gNO produced by the administered dosage of NORS. In an alternative embodiment, the NORS can be acidified beforehand and administered at a later time. In some embodiments a mechanism or device for storing or otherwise formulating an acidified NORS can be used which preserves its potency and therapeutic effect when administered.

For example, by introducing sodium nitrite (or other salts of nitrites) to a saline solution it will very slowly produce nitric oxide gas, but in an undetectable amount (as measured by chemiluminescence analysis methodology (ppb sensitivity)). The rate of NO produced from the solution increases as pH decreases. The rate of gNO production increases significantly once the solution's pH is below 4.0. In one embodiment, where an acidifying gas is used to lower the NORS pH, such as an acidifying NO gas, an unexpected result is that the amount of NO gas evolving from (i.e. coming out of) the NORS is more than the amount of NO gas added. Generally, NO is produced based on the following equilibrium equations:

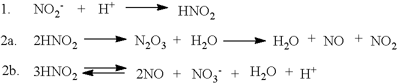

The acid (e.g. from the acidifying agent) donates the H⁺ to the nitrite (NO₂⁻). The amount of H⁺ present, affects the rate at which the reaction moves towards HNO₂. Additionally, NO production is facilitated by HNO₂ concentration. As can be seen from these equations, increasing the concentration of nitrites (i.e. NO releasing compound or agent) present in the NORS (for example 60 mM versus 20 mM), requires more acid to achieve the same pH. In other words, the more HNO₂ produced, the lower the pH will be. Interestingly, either NO₂ or pH alone, even at optimal levels, is insufficient to have an antimicrobial, or therapeutic effect on a subject.

Some example embodiments can be used to help illustrate the need for increasing the acid concentration in a NORS to achieve an equivalent pH as the nitrite concentration increases. In one example embodiment, a pH of from about 3.45 to about 3.65 can be achieved in saline solution including about 20 mM nitrites (about 0.09% w/v) by adding a sufficient amount of citric acid to achieve a concentration of about 7.3 mM (about 0.14% w/v). At a higher concentration of nitrites, such as 60 mM nitrites (about 0.28% w/v), a sufficient amount of citric acid can be added to the saline solution to achieve a concentration of 15.6 mM (about 0.3% w/v) in order to reduce the pH to between about 3.45 and 3.65. At a concentration of 100 mM nitrites (about 0.46% w/v), a sufficient amount of citric acid can be added to the saline solution to achieve a concentration of 36 mM (about 0.7% w/v) in order to reduce the pH to between about 3.45 and 3.65.

In yet another example embodiment, ascorbic acid can be used to illustrate the need for more acid as the concentration of nitrites increases. Specifically, a sufficient amount of ascorbic acid can be added to a saline solution including 20 mM nitrites (about 0.09% w/v) to achieve a concentration of 127 mM (about 2.25% w/v) ascorbic acid in order to reduce the pH to between about 3.45 and 3.65. At a higher concentration of nitrites, such as 60 mM nitrites (about 0.28% w/v), a sufficient amount of ascorbic acid can be added to the saline solution to achieve a concentration of 352 mM (about 6.2% w/v) in order to reduce the pH to between about 3.45 and 3.65. At a concentration of 100 mM nitrites (about 0.46% w/v), a sufficient amount of ascorbic acid can be added to the saline solution to achieve a concentration of 545 mM (about 9.6% w/v) in order to reduce the pH to between about 3.45 and 3.65.

In a more specific embodiment, using a particular nitric oxide releasing compound, a pH of from about 3.45 to about 3.65 can be achieved in saline solution including about 20 mM sodium nitrate (about 0.14% w/v) by adding a sufficient amount of citric acid to achieve a concentration of about 7.3 mM (about 0.14% w/v). At a higher concentration of sodium nitrite, such as 60 mM sodium nitrite (about 0.4% w/v), a sufficient amount of citric acid can be added to the saline solution to achieve a concentration of 15.6 mM (about 0.3% w/v) in order to reduce the pH to between about 3.45 and 3.65. At a concentration of 100 mM sodium nitrite (about 0.7% w/v), a sufficient amount of citric acid can be added to the saline solution to achieve a concentration of 36 mM (about 0.7% w/v) in order to reduce the pH to between about 3.45 and 3.65.

In yet another specific embodiment, a sufficient amount of ascorbic acid can be added to a saline solution including 20 mM sodium nitrite (about 0.14% w/v) to achieve a concentration of 127 mM (about 2.25% w/v) ascorbic acid in order to reduce the pH to between about 3.45 and 3.65. At a higher concentration of sodium nitrite, such as 60 mM sodium nitrite (about 0.4% w/v), a sufficient amount of ascorbic acid can be added to the saline solution to achieve a concentration of 352 mM (about 6.2% w/v) in order to reduce the pH to between about 3.45 and 3.65. At a concentration of 100 mM sodium nitrite (about 0.7% w/v), a sufficient amount of ascorbic acid can be added to the saline solution to achieve a concentration of 545 mM (about 9.6% w/v) in order to reduce the pH to between about 3.45 and 3.65.

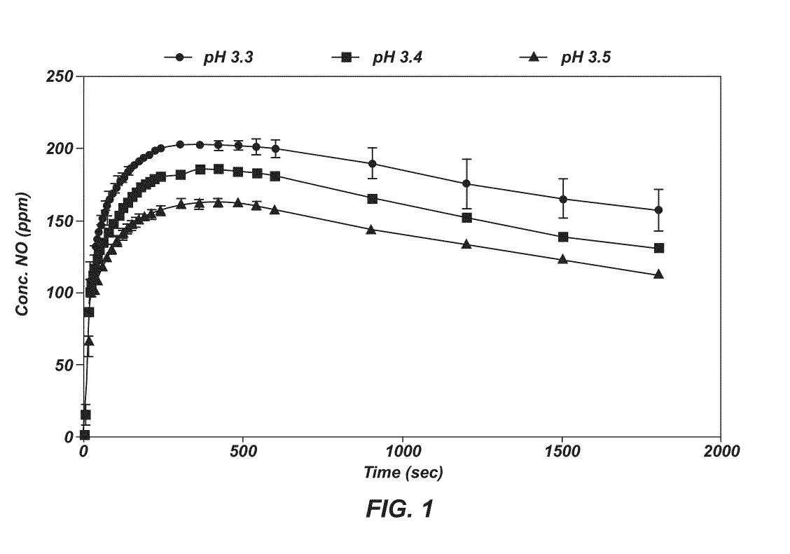

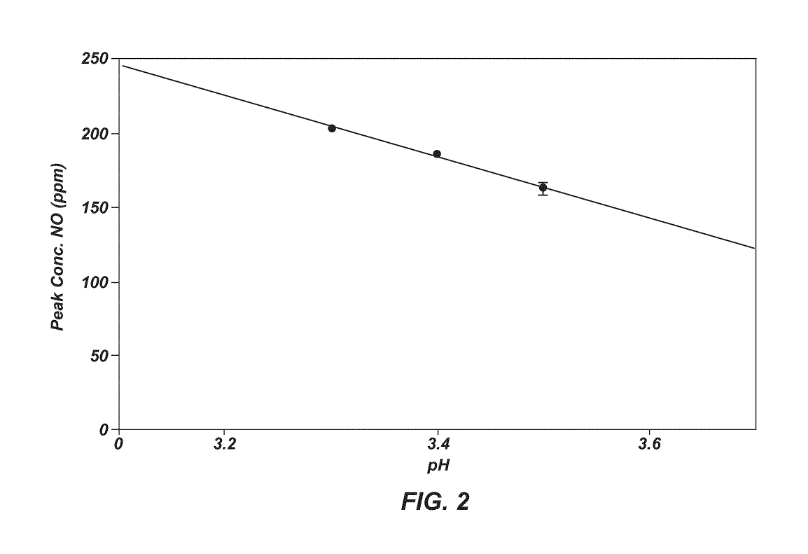

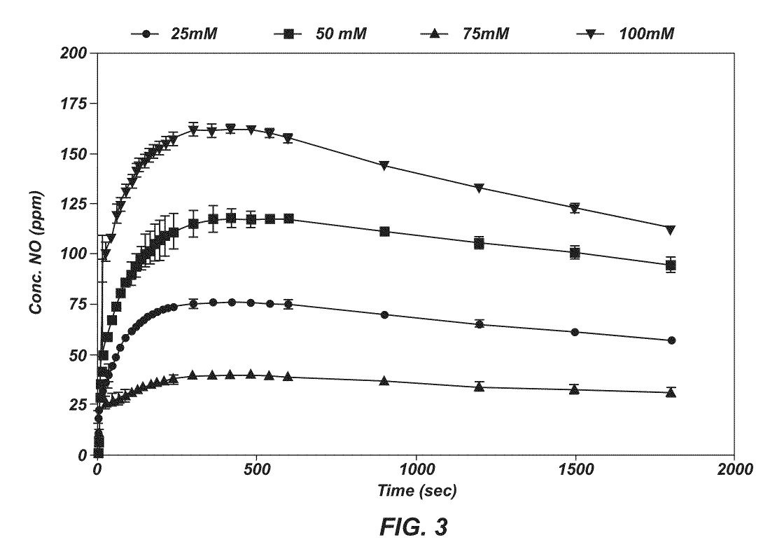

The effect of pH on gNO production is further illustrated in FIG. 1. FIG. 1 shows a comparative analysis between NORS compositions prepared at the same concentration (100 mM) but at differing pH. As can be seen, decreasing pH values results in increased gNO production. Additionally, as shown in FIG. 2, peak gNO production increases with decreasing pH. Alternatively, increasing concentrations of nitrite in NORS also increases initial gNO production, but also results in a greater drop in gNO release over time. This is shown in FIG. 3, where various NORS samples were prepared at differing concentrations but at constant pH (3.45). Some exemplary results are illustrated below in Table 1, as used to determine the rate constant k′ for the production of gNO.

TABLE 1
Determination of Rate Constant (K′) for Production of gNO

| [NO₂⁻] | | [NO]ₛₛ | δ[NO]ₛₛ | [NO]ₛₛ | δ[NO]ₛₛ | k′ | Δk′ |
|---|---|---|---|---|---|---|---|
| (mM) | pH | (ppm) | (ppm) | (mM) | (mM) | (min⁻¹) | (min⁻¹) |
| 100 | 3.3 | 203.1 | 1.3 | 0.008290 | 5E−05 | 1.654 | 0.010 |
| 100 | 3.4 | 186.5 | 0.1 | 0.007612 | 4E−06 | 1.912 | 0.001 |
| 100 | 3.5 | 162.1 | 3.1 | 0.006616 | 1E−4 | 2.092 | 0.040 |
| 25 | 3.45 | 40.2 | 0.2 | 0.001639 | 8E−06 | 1.850 | 0.009 |
| 50 | 3.45 | 76.3 | 1.2 | 0.003112 | 5E−05 | 1.754 | 0.027 |
| 75 | 3.45 | 118.0 | 4.5 | 0.004816 | 2E−4 | 1.810 | 0.069 |

| | | |
|---|---|---|
| Average | 1.845 | 0.026 |

Based on the results above, k′ can be assumed to be roughly equal to 1.85±0.03 (min⁻¹). Therefore, the rate of NO production from a NORS with nitrite concentration between 25 mM to 100 mM and pH 3.2 to 3.7 is given by equation 3 below, where d[NO]/dt is in mM/min and [NO₂⁻] is in mM:

$$\frac{d [ NO ]}{d t} = 1.85 ( \pm 0.03 ) [ {NO}_{2}^{-} ] {10}^{- p H} \qquad ( 3 )$$

Hence, in some embodiments, the NORS can be configured or prepared to provide an initial burst or dose of gNO, followed by an extended release thereof at a therapeutically effective amount. In some embodiments, the gNO released can be according to a desired or pre-designed profile. In one embodiment, the profile can specify the release of gNO in an amount that is the equal to the amount released initially over the course of the period. In another aspect the profile can specify release of gNO in an amount that decreases as compared to the amount released initially over the course of the period. In some embodiments, the decrease may be from 10% to 90% over the specified period. In other embodiments, the decrease may be from about 30% to about 80% over the specified period. In a further aspect, the decrease may be from about 40% to about 70% over the specified period. In an additional aspect, the decrease may be about 50% over the specified period. In some embodiments, the period can be from about 5 minutes to about 1 week. In another embodiment, the specified period can be from about 1 day to about 1 week. In a further embodiment the specified period can be from about 5 minutes to about 24 hours. In yet an additional embodiment, the specified period can be from about 5 minutes to about 12 hours. In another embodiment, the specified period can be from about 5 minutes to about 1 hour. In another embodiment, the specified period can be from about 5 minutes to about 30 minutes.

The NORS may be administered as a controlled or extended release formulation of gNO, and optionally with a carrier formulation (i.e. a carrier other than water). In one aspect, the extended release may release an effective amount of gNO from the formulation at a controlled rate such that therapeutically effective levels (but below toxic levels) of the component are maintained over an extended period of time. In one aspect, the period of time may range from about 1 minute to about 24 hours. In another aspect, the period of time can be greater than 30 minutes. In another aspect, the period of time can range from about 30 to about 60 minutes. In another aspect, the time period may be several hours. In another embodiment, the period of time can be from about 10 to about 45 minutes. In yet a further embodiment, the time period may be at least 15 minutes. In one embodiment, the time period can be at least 30 minutes. In one embodiment, the time period can be at least 8 hours. In another embodiment, the time period can be at least 12 hours. In one aspect, such interval or period may be about or at least 4 hours, about 8 hours, about 12 hours, from about 2 hours to about 12 hours, or other suitable time period. Thus, the administered NORS provides for continuous or otherwise extended delivery of NO to the treatment site of the subject, or a location distal therefrom. Further, the amount of administered NORS may be varied in order to optimize the duration of NO production and delivery.

Some invention embodiments provide a method of treating a subject by delivering a NORS to a treatment site of the subject and allowing gNO to be produced by the NORS. In one aspect, the amount of gNO released is a therapeutically effective amount. In some aspects, the amount of NORS delivered may be selected in order to allow a specific amount of gNO to be released. In certain embodiments, the NORS can be prepared just prior to administration (e.g. within 5 or 10 minutes) to the subject by activating a dormant NORS with an acidifying agent. For example, as described elsewhere herein, an organic acid may be added to the dormant NORS, such as citric acid. In other embodiments, an acidifying gas, such as NO or NO₂ containing gas can be used. Once the acidifier is added to the dormant NORS the NORS is activated and can be administered. As previously mentioned, the activated NORS can provide for extended production of NO. Further, in some embodiments, the acidifying agent can be added from about 2 seconds to about 2 hours prior to administration of the NORS to a subject. In another aspect, the acidifying agent can be added from about 1 minute to about 1 hour prior to administration of the NORS to a subject. In an additional aspect, the acidifying agent can be added from about 4 to about 48 hours prior to administration. In a further aspect, the acidifying agent may be added at administration of the NORS to a subject. In a further embodiment, the acidifying agent may be added following administration of the NORS, or of a gNO releasing compound or agent to a subject. In this embodiment, the NORS solution is formed in-vivo and releases gNO thereafter.

In some embodiments, the NORS may be reapplied (ie administered) one or more times, as necessary to provide effective treatment. For example, administration to the nares or surrounding areas can occur as a single dose or as multiple doses at one or more specified locations as part of a treatment regimen. In one aspect the interval can be about once every day. In another aspect, the interval can be about once every 7 days. In another aspect, the interval can be about once every 14 days. In a further aspect, the interval can be about once every 28 or 30 days. In another aspect, the interval can be from about every hour to every 28 days. Other suitable intervals can also be used, such as every 1-12 months. Nearly any interval identified as particularly effective in treating a given condition or disorder can be used.

The present technology allows for delivery of NO to an ambulatory subject. For example, the extended production and delivery of NO to the treatment site by way of the administered NORS allows for the treated subject to remain ambulatory during treatment. Thus, the subject is not constrained to a nitric oxide delivery device during the entire duration of NO delivery. The present method of treatment can be suitable for all subjects that are commercially-salable. Furthermore, the current method of treatment can use gNO as any one of a microbicidal agent, a bactericidal agent, and a virucidal agent, or combinations thereof.

When properly implemented, the current methods can reduce the occurrence of a respiratory condition in a subject or group of subjects. In one aspect, the metaphylactic implementation of this method can reduce the occurrence of a respiratory condition amongst a group of subjects by greater than 20%. In another aspect, the metaphylactic implementation of this method can reduce the occurrence of a respiratory condition amongst the group by greater than 40%. In another aspect, the prophylactic treatment can reduce the occurrence of a respiratory condition in a subject by greater than 20%. In another aspect, the prophylactic treatment of a subject via the current method can reduce the occurrence of a respiratory condition by greater than 40%. In another aspect, the implementation of this method can delay the occurrence or onset of the respiratory condition by at least 3 days. In another aspect, the implementation of this method can delay the occurrence or onset of the respiratory condition by at least 7 days. In another aspect, the implementation of this method can delay the occurrence or onset of the respiratory condition by at least 14 days.

Additionally, the method can increase blood Methemoglobin (MetHg) levels of the subject upon administration of gNO, which can indicate delivery and bioavailability of gNO to the lungs of the subject. Hence, MetHG can be an indicator of bioavailability of administered gNO in the subject. Furthermore, the method can allow the blood MetHg levels to return to about pre-treatment levels within about 60 minutes of administration. In one aspect, the method can allow the blood MetHg levels to return to about pre-treatment levels within about 120 minutes of administration. In one aspect the method can allow the blood MetHG levels to return to about pre-treatment levels within about 90 minutes. In one aspect, the method can allow the blood MetHg levels to return to about pre-treatment levels within about 30 minutes of administration.

Likewise, the method can increase the fraction of exhaled nitric oxide of the subject upon administration of gNO, which can also indicate delivery of gNO to the lungs of the subject. Hence, fraction of exhaled nitric oxide can be an indicator of bioavailability of administered gNO in the subject. The method can allow the fraction of exhaled nitric oxide to return to about pre-treatment levels within about 120 minutes of administration. In one aspect, the method can allow the fraction of exhaled nitric oxide to return to about pre-treatment levels within about 90 minutes of administration. In another aspect, the method can allow the fraction of exhaled nitric oxide to return to about pre-treatment levels within about 60 minutes of administration. In another aspect, the method can allow the fraction of exhaled nitric oxide to return to about pre-treatment levels within about 30 minutes of administration.

Similarly, the method can increase blood nitrite levels of the subject upon administration of gNO, which can also indicate delivery of gNO to the lungs of the subject. Hence, blood nitrite levels can be an indicator of bioavailability of administered gNO in the subject. Additionally, the method can allow the blood nitrite levels to return to about pre-treatment levels within about 60 minutes of administration. In one aspect, the method can allow the blood nitrite levels to return to about pre-treatment levels within about 120 minutes of administration. In one aspect, the method can allow the blood nitrite levels to return to about pre-treatment levels within about 90 minutes of administration. In one aspect, the method can allow the blood nitrite levels to return to about pre-treatment levels within about 30 minutes of administration.

Furthermore, with the current method, the administration of gNO or NORS does not substantially affect blood cortisol levels in the subject, which can be an indicator of additional stress caused by the gNO or NORS themselves. Also with the current method, the nitrite levels in meat obtained from commercially-salable animals are substantially unchanged post-treatment with gNO or NORS. This can be a significant advantage over anti-biotic treatments, which often require a waiting period after treatment before the animal can be used for meat production.

In another embodiment, another method of treating bovine respiratory disease in a subject is described. This method can include administering a therapeutically effective amount of gNO to the subject, wherein at least one treatment outcome after treatment with gNO is equivalent to the treatment outcome found after treatment with dose, or comparable dose of tilmicosin or equivalent antibiotic. It is to be understood that while various methods of treatment are set forth expressly herein, that support for corresponding secondary use claims is afforded thereby. For example, “a method of treating bovine respiratory disease in a veterinary subject, comprising administering a therapeutically effective amount of gaseous nitric oxide (gNO) to the subject from a nitric oxide releasing solution (NORS), wherein at least one treatment outcome after treatment with gNO is equivalent to the treatment outcome found after treatment with tilmicosin” can provide express support for “use of a nitric oxide releasing solution (NORS) for the manufacture of a veterinary medicament for treatment of a respiratory disease or disorder in a subject, wherein the NORS releases a therapeutically effective amount of gaseous nitric oxide (gNO), and wherein at least one treatment outcome resulting from treatment with the NORS is equivalent to the treatment outcome resulting from treatment with tilmicosin.”

In one embodiment, the subject can have a respiratory disease or condition, or a propensity to obtain a respiratory disease or condition. Suitable subjects can include subjects listed in other embodiments, aspects, and examples described herein. Similarly, administration and therapeutically effective amounts can include any suitable administration and therapeutically effective amount, including methods of administration and therapeutically effective amounts in other embodiments, aspects, and examples described herein.

Tilmicosin or an equivalent antibiotic can be used to prophylactically or metaphylactically treat BRDc and other respiratory conditions. Such treatment is generally performed with the expectation that the subject treated with the antibiotic will not suffer reduced weight gain, morbidity, and death, but will experience an improved outcome in all of these areas. Hence, the outcome of the current method can be the same as, or effectively the same as, or otherwise similar to any outcome obtained by treatment with tilmicosin or equivalent antibiotic. In some embodiments, such an outcome resulting from the treatment with gNO is equivalent or non-inferior to the outcome resulting from the treatment with tilmicosin or equivalent antibiotic. In one aspect, the equivalent or non-inferior treatment outcome of this method can include at least one of weight gain, morbidity, mortality, and combinations thereof. In one aspect, the equivalent or non-inferior treatment outcome can include weight gain. In one aspect, the equivalent or non-inferior treatment outcome can include morbidity. In one aspect, the equivalent or non-inferior treatment outcome can include mortality.

Tilmicosin dosages can range depending on the body weight of the subject. In one aspect the tilmicosin dosage can be from about 10 mg to about 20 mg per kg of subject body weight. In another aspect, the tilmicosin dosage can be from about 11 mg to about 15 mg per kg of subject body weight. In yet another aspect, the tilmicosin dosage can be from about 12 mg to about 14 mg per kg of subject body weight.

Any number of devices can be used to administer the NORS to the nares of a subject, such as a bovine. Generally, such devices will include a reservoir for holding NORS (in either an activated or deactivated state) and an applicator fluidly coupled to the reservoir for directing the NORS to the desired location of application or treatment site (i.e. a nasal cavity of a subject). Further such a device will include a mechanism for driving or otherwise moving or flowing NORS from the reservoir and out of the applicator, such as a pump, or other suitable device.

The current technology can be illustrated by a number of non-exclusive example embodiments as follows:
In one example, a liquid nitric oxide releasing solution (NORS) for veterinary use is described, which comprises at least one nitric oxide releasing compound and at least one acidifying agent, wherein the NORS provides an extended release of a therapeutically effective amount of nitric oxide gas (gNO).
In one example, the at least one nitric oxide releasing compound is selected from the group consisting of a nitrite, a salt thereof, or any combination thereof.
In one example, the nitric oxide releasing compound is sodium nitrite.
In one example, the amount of the at least one nitric oxide releasing compound is less than about 0.5% w/v.
In one example, the at least one acidifying agent is an acid.
In one example, the acid is citric acid.
In one example, the amount of the at least one acidifying agent is less than about 0.5% w/v.
In one example, the therapeutically effective concentration of gNO is about 160 ppm.
In one example, the NORS is a saline-based solution.
In one example, the gNO is released over a period of at least 30 minutes.
In one example, the gNO is released over a period of at least 4 hours.
In one example, the gNO is released over a period of at least 8 hours.
In one example, the gNO is released over a period of at least 12 hours.
In one example, the gNO is released over a period of at least 24 hours.
In one example, a liquid nitric oxide releasing solution (NORS) for veterinary use is described, comprising at least one nitric oxide releasing compound and at least one acidifying agent, wherein either the at least one nitric oxide releasing compound or the at least one acidifying agent is present at a concentration of less than about 0.5% w/v, and wherein the NORS releases a therapeutically effective amount of nitric oxide gas (gNO).
In one example, the at least one nitric oxide releasing compound is selected from the group consisting of a nitrite, a salt thereof, or any combination thereof.
In one example, the nitric oxide releasing compound is sodium nitrite.
In one example, the at least one acidifying agent is an acid.
In one example, the acid is citric acid.
In one example, the concentration of the at least one acidifying agent is less than about 0.5% w/v.
In one example, the concentration of the at least one nitric oxide releasing compound is less than about 0.5% w/v.
In one example, the concentrations of both the at least one nitric oxide and the at least on acidifying agent are less than about 0.5% w/v.
In one example, the therapeutically effective concentration of gNO is about 160 ppm.
In one example, the NORS is a saline-based solution.
In one example, a method for the treatment of a disease or disorder in a veterinary subject is described, comprising administering to the subject a liquid nitric oxide releasing solution (NORS) having at least one nitric oxide releasing compound and at least one acidifying agent, wherein either the at least one nitric oxide releasing compound or the at least one acidifying agent is present at a concentration of less than 0.5% w/v, and wherein the NORS releases a therapeutically effective amount of nitric oxide gas (gNO).
In one example, the disease or disorder is a respiratory disease or disorder.
In one example, the concentration of the at least one nitric oxide releasing compound is less than about 0.5% w/v.
In one example, the concentration of the at least one acidifying agent is less than about 0.5% w/v.
In one example, the concentrations of both the at least one nitric oxide releasing compound and the at least one acidifying agent are less than about 0.5% w/v.
In one example, the NORS is administered as a spray.
In one example, the NORS is administered intranasally to the veterinary subject.
In one example, the veterinary subject is a bovine.
In one example, the disease or disorder is Bovine Respiratory Disease Complex (BRDc).
In one example, administration of the NORS at an administration situs provides a therapeutic effect at a situs distal from the administration situs.
In one example, a method of treating a respiratory condition in a veterinary subject is described, comprising administering a therapeutically effective amount of gaseous nitric oxide (gNO) to the subject from a nitric oxide releasing solution (NORS) deposited in the subject's nose, wherein occurrence of the respiratory condition is reduced.
In one example, the subject is a bovine.
In one example, the respiratory condition includes at least one of bovine respiratory disease (BRDc), bovine herpes virus 1 (BVH-1), bovine respiratory syncytial virus (BRSV).
In one example, the administration is metaphylactic.
In one example, the administration is prophylactic.
In one example, the NORS is dispensed into the subject's nostrils.
In one example, the NORS enters the subject's nasal cavity.
In one example, the NORS enters the subject's sinus cavity.
In one example, the NORS has a nitrite content of from about 51 mM to about 100 mM.
In one example, the amount of NORS deposited is from about 1 to about 100 ml.
In one example, the amount of NORS deposited is from about 1 to about 50 ml.
In one example, the amount of NORS deposited is about 32 ml.
In one example, the NORS has an acid content of from about 5 mM to about 600 mM.
In one example, the acid is citric acid.
In one example, the acid content is from about 5 mM to about 50 mM.
In one example, the acid is ascorbic acid.
In one example, the acid content is from about 100 mM to about 600 mM.
In one example, about 40% to about 100% of the NORS is deposited beyond the nasal vestibule.
In one example, about 40% to about 80% of the NORS is deposited beyond the nasal vestibule.
In one example, about 40% to about 60% of the NORS is deposited beyond the nasal vestibule.
In one example, the therapeutically effective amount is from about 40 to about 400 ppm gNO.
In one example, the therapeutically effective amount is from about 100 to about 220 ppm gNO.
In one example, the therapeutically effective amount is about 160 ppm gNO.
In one example, the occurrence of a respiratory condition is reduced by greater than about 40%.
In one example, the occurrence of a respiratory condition is delayed by at least 7 days.
In one example, the occurrence of a respiratory condition is delayed by at least 10 days.
In one example, the occurrence of a respiratory condition is delayed by at least 14 days.
In one example, blood MetHg levels of the subject increase upon administration.
In one example, blood MetHg levels return to about pre-treatment levels within about 60 minutes of administration.
In one example, blood MetHg levels return to about pre-treatment levels within about 30 minutes of administration.
In one example, blood nitrites of the subject increase upon administration.
In one example, blood nitrites return to about pre-treatment levels within about 60 minutes of administration.
In one example, blood nitrites return to about pre-treatment levels within about 30 minutes of administration.
In one example, a fraction of exhaled nitric oxide (F_{E}NO) is increased upon administration.
In one example, gNO or NORS do not substantially affect blood cortisol levels in the subject.
In one example, the veterinary subject is a commercially-salable animal.
In one example, nitrite levels in meat obtained from the commercially-salable animal are substantially unchanged post-treatment.
In one example, gNO is a microbicidal agent.
In one example, gNO is a bactericidal agent.
In one example, gNO is a virucidal agent.
In one example, a method of treating bovine respiratory disease in a veterinary subject is described, comprising administering a therapeutically effective amount of gaseous nitric oxide (gNO) to the subject from a nitric oxide releasing solution (NORS), wherein at least one treatment outcome after treatment with gNO is equivalent to the treatment outcome found after treatment with tilmicosin.
In one example, a dosage of tilmicosin is from about 10 mg to about 20 mg per kg of subject body weight.
In one example, the dosage of tilmicosin is from about 11 mg to about 15 mg per kg of subject body weight.
In one example, the dosage of tilmicosin is from about 12 mg to about 14 mg per kg of subject body weight.
In one example, the treatment outcome includes at least one of subject weight gain, morbidity, mortality, or a combination thereof.
In one example, the treatment outcome includes subject weight gain.
In one example, the treatment outcome includes subject morbidity.
In one example, the treatment outcome includes subject mortality.
In one example, a method of treating a respiratory condition in a veterinary subject is described, comprising administering a therapeutically effective amount of gaseous nitric oxide (gNO) to the subject from a nitric oxide releasing solution (NORS) administered to the subject's nose, wherein at least one indicator of bioavailability of the administered gNO in the subject is increased.
In one example, the NORS is deposited in the nose of the subject.
In one example, the NORS is deposited on the nose of the subject.
In one example, the NORS is deposited both on and in the nose of the subject.
In one example, the at least one indicator of bioavailability of gNO includes at least one of increased MetHg levels, increased blood nitrate levels, increased fraction of exhaled gNO, or combinations thereof as compared to levels present in the subject prior to NORS administration.
In one example, the at least one indicator of bioavailability of gNO includes increased MetHg levels.
In one example, the at least one indicator of bioavailability of gNO includes increased blood nitrate levels.
In one example, the at least one indicator of bioavailability of gNO includes increased F_{E}NO.
In one example, the at least one indicator of bioavailability of gNO returns to about baseline levels within about 90 minutes of administration of the NORS.
In one example, the at least one indicator of bioavailability of gNO returns to about baseline levels within about 60 minutes of administration of the NORS.
In one example, the at least one indicator of bioavailability of gNO returns to about baseline levels within about 30 minutes of administration of the NORS.
In one example, the respiratory condition includes inflammation of the upper respiratory tract.
In one example, the respiratory condition includes a microbial infection.
In one example, the microbial infection includes at least one of a viral infection and a bacterial infection.
In one example, the microbial infection includes a viral infection.
In one example, the microbial infection includes a bacterial infection.
In one example, a method of administering a nitric oxide releasing solution (NORS) to a veterinary subject is described, comprising:
   providing a nitric oxide releasing agent;
   providing an acidifying agent;
   combining the nitric oxide releasing agent and the acidifying agent to provide an activated NORS up to 24 hours before administration; and
   administering the activated NORS to the subject.
In one example, the NORS is activated up to 8 hours before administration.
In one example, the NORS is activated up to 1 hour before administration.
In one example, the NORS is activated up to 30 minutes before administration.
In one example, the NORS is activated up to 10 minutes before administration.
In one example, the NORS is activated up to 5 minutes before administration.
In one example, the NORS is activated up to 1 minute before administration.
In one example, the NORS is activated during administration.
In one example, a nitric oxide releasing solution (NORS) for veterinary use is described, comprising at least one nitric oxide releasing agent and at least one acidifying agent, wherein the NORS provides a therapeutically effective amount of gaseous nitric oxide (gNO), and wherein the NORS has an antimicrobial effect on Mannheimia haemolytica.
In one example, the NORS has an antimicrobial effect on at least one of Infectious Bovine Rhinotracheitis (IBR), Bovine Respiratory Syncytial Virus (BRSV), and Bovine Parainfluenza-3 (PI-3).
In one example, the nitric oxide releasing agent is present at a concentration of from about 51 mM to about 100 mM.
In one example, the nitric oxide releasing agent is present at a concentration of less than 0.5% w/v.
In one example, the therapeutically effective amount of gNO is from about 100 ppm to about 220 ppm.
In one example, the therapeutically effective amount of gNO is about 160 ppm.
In one example, the acidifying agent is present at a concentration of from about 5 mM to about 600 mM.
In one example, the acidifying agent is present at a concentration of less than 0.5% w/v.
In one example, a nitric oxide releasing solution (NORS) for veterinary use is described, comprising at least one nitric oxide releasing compound and at least one acidifying agent, wherein the NORS releases a therapeutically effective amount of gaseous nitric oxide (gNO) that is less than 160 ppm for an extended period.
In one example, the therapeutically effective amount of gNO is from about 40 ppm to about 155 ppm.
In one example, the therapeutically effective amount of gNO is from about 60 ppm to about 120 ppm.
In one example, the therapeutically effective amount of gNO is from about 80 ppm to about 100 ppm.
In one example, the therapeutically effective amount of gNO is released over a period of at least 30 minutes.
In one example, the therapeutically effective amount of gNO is released over a period of at least 4 hours.
In one example, the therapeutically effective amount of gNO is released over a period of at least 8 hours.
In one example, the therapeutically effective amount of gNO is released over a period of at least 12 hours.
In one example, the therapeutically effective amount of gNO is released over a period of at least 24 hours.
In one example, a nitric oxide releasing solution (NORS) for veterinary use is described, comprising at least one nitric oxide releasing compound and at least one acidifying agent, wherein the NORS releases a therapeutically effective amount of gaseous nitric oxide (gNO) for longer than 30 minutes.
In one example, the therapeutically effective amount of gNO is from about 40 ppm to about 400 ppm.
In one example, the therapeutically effective amount of gNO is from about 100 ppm to about 220 ppm.
In one example, the therapeutically effective amount of gNO is about 160 ppm.
In one example, use of a nitric oxide releasing solution (NORS) for the manufacture of a veterinary medicament for treatment of a respiratory disease or disorder in a subject is described, wherein the NORS releases a therapeutically effective amount of gaseous nitric oxide (gNO), and wherein at least one treatment outcome resulting from treatment with the NORS is equivalent to the treatment outcome resulting from treatment with tilmicosin.
In one example, a dosage of tilmicosin is from about 10 mg to about 20 mg per kg of subject body weight.
In one example, the dosage of tilmicosin is from about 11 mg to about 15 mg per kg of subject body weight.
In one example, the dosage of tilmicosin is from about 12 mg to about 14 mg per kg of subject body weight.
In one example, the treatment outcome includes at least one of subject weight gain, morbidity, mortality, or a combination thereof.
In one example, the treatment outcome includes subject weight gain.
In one example, the treatment outcome includes subject morbidity.
In one example, the treatment outcome includes subject mortality.
In one example, use of a nitric oxide releasing solution (NORS) for the manufacture of a veterinary medicament for treatment of a respiratory disease or disorder is described, wherein the NORS releases a therapeutically effective amount of gaseous nitric oxide (gNO), and wherein the NORS is deposited simultaneously in both nostrils of a subject.
In one example, the NORS has a nitrite content of from about 51 mM to about 100 mM.
In one example, the amount of NORS deposited in both nostrils is from about 1 to about 100 ml.
In one example, the amount of NORS deposited in both nostrils is from about 1 to about 50 ml.
In one example, the amount of NORS deposited in both nostrils is about 32 ml.
In one example, the therapeutically effective amount of gNO is from about 40 ppm to about 400 ppm.
In one example, the therapeutically effective amount of gNO is from about 100 ppm to about 220 ppm.
In one example, the therapeutically effective amount of gNO is about 160 ppm.
In one example, use of a nitric oxide releasing solution (NORS) in the manufacture of a veterinary medicament for the treatment of a disease or disorder in a subject is described, wherein a nitric oxide releasing agent and an acidifying agent are combined to provide an activated NORS up to 24 hours prior to administration, and wherein the activated NORS is administered to the subject.
In one example, the NORS is activated up to 8 hours prior to administration.
In one example, the NORS is activated up to 1 hour prior to administration.
In one example, the NORS is activated up to 30 minutes prior to administration.
In one example, the NORS is activated up to 10 minutes prior to administration.
In one example, the NORS is activated up to 5 minutes prior to administration.
In one example, the NORS is activated up to 1 minute prior to administration.
In one example, the NORS is activated during administration.

### EXAMPLES

Certain invention embodiments are further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

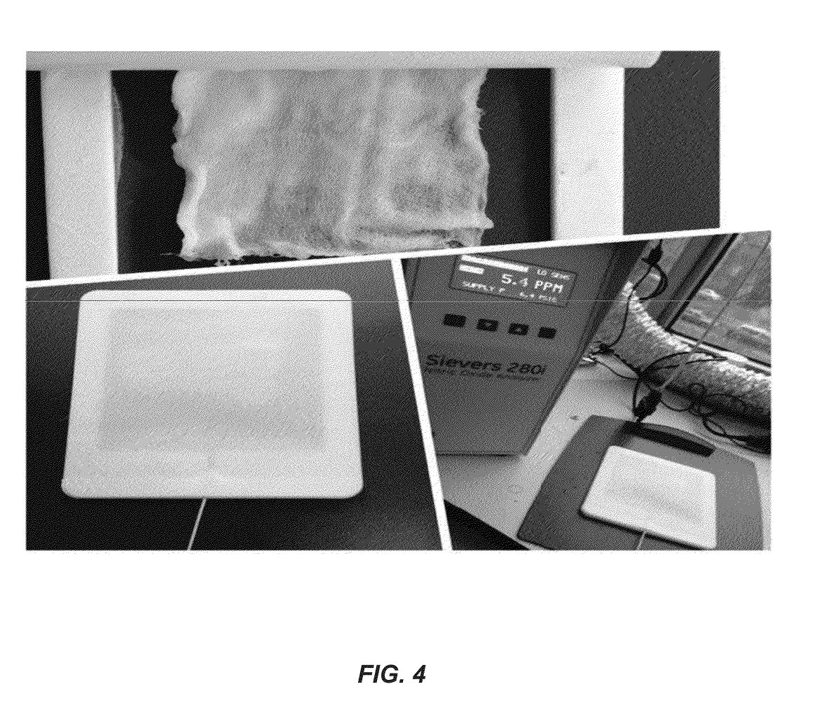

A NORS was prepared at a nitrite strength of 0.3% w/v and pH 3.7. Once ready, a 3×3 inch gauze was dipped into the solution, lightly squeezed to discard excess liquid and placed in a “Hath Bath” device (FIG. 4). At different time points, the gNO that was being released was measured with a chemiluminescence analyzer.

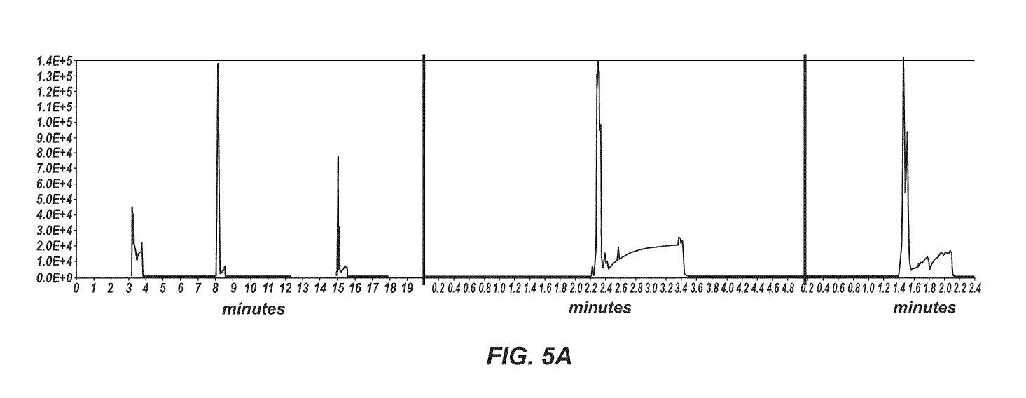

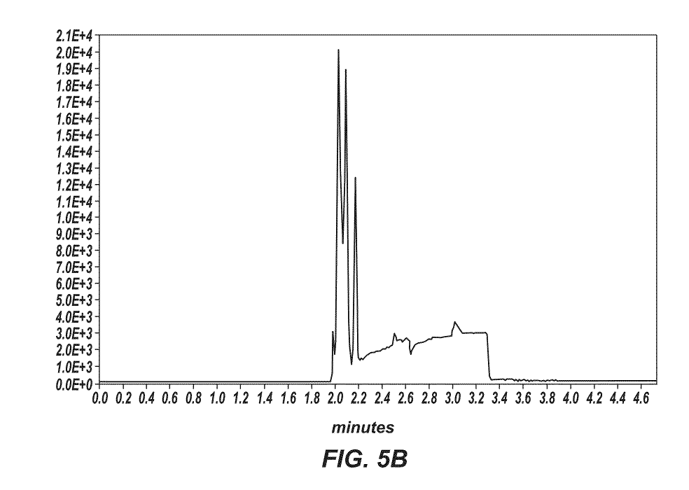

FIG. 5 shows the amount of gNO detected at 3, 8, and 15 minutes as well as 3, 4 (5A), and 24 (5B) hours. The X scale is Time (minutes) from start of measuring point (showing pre-measuring amount as 0-0.1 ppm) and Y scale showing amount of NO (measured in ppb).

The Chemiluminescent analyzer has a sample draw rate of 200 cc per min and thus, there is an initial peak and reduction in NO concentration following that. The “Hathback” may not be completely sealed and thus some NO may “escape”. However, release of NO was still detected 24 hours after gauze was saturated with the NORS solution.

### Example 2

All of the following bacteria are common in wound infections:

A. baumannii is a species of pathogenic bacteria, referred to as an aerobic gram-negative bacterium, which is resistant to most antibiotics.

E. coli—gram negative, common bacteria.

S. aureus is a common cause of surgical-site infection. It's a gram positive and it is frequently part of skin flora.

Methicillin-Resistant Staphylococcus aureus—MRSA is, by definition, a S. aureus bacteria that has developed resistance to beta-lactam antibiotics which include the penicillins (methicillin, dicloxacillin, nafcillin, oxacillin, etc.) and the cephalosporins.

A. baumanii, MRSA, and E. coli bacterial culture were obtained from American Type Culture Collection (ATCC #BAA-747, #700698 and #25922). Bacteria were grown in Lysogeny broth (LB) (E. coli and A. baumanii) or Brain-Heart Infusion Broth (BHI) (MRSA) to 0.5 McFarland standard. 1 mL aliquots of these preparations containing approximately 2.5×10⁸ cfu/mL were stored at −70° C. On the day of the experiments the fresh stock was removed from the freezer, thawed, and 2 mL of LB or BHI was added. Cultures were further diluted with LB or BHI to 10⁶ colony forming units per milliliter (cfu/mL).

NORS was prepared by mixing a specific concentration of sodium nitrite (0.07-0.41%) in saline and then reducing the pH to 3.7 with citric acid. Controls were—saline, sodium nitrite at 0.41% and pH of 6, and saline at pH 3.7 (reduced with citric acid).

100 μl of bacteria (10⁶ cfu/mL) was mixed with 900 μl NORS. After 10 min, samples were serially diluted and plated on either LB or BHI agar plates. Cultures were incubated at 37° C. overnight (0/N) and then cfus were counted to quantify bacterial growth. Each experiment was done in triplicate and each experiment repeated three times.

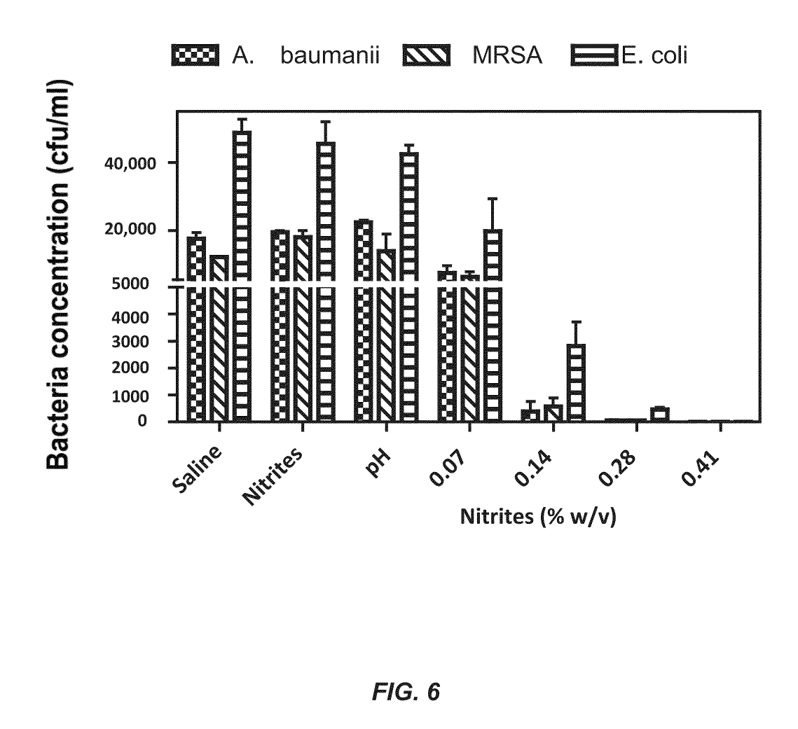

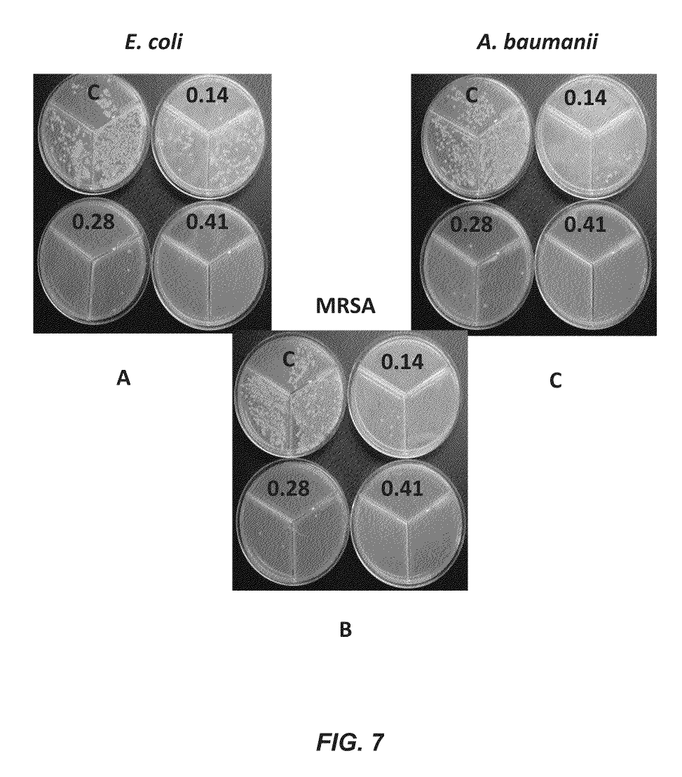

NORS included citric acid and each with a different concentration of nitrites in saline solution were tested on 3 different bacteria species (A. baumanii, MRSA and E. coli) at 10 min exposure time in order to evaluate antibacterial efficacy of the NORS. 0.41% nitrites at pH 3.7 (0.2% w/v citric acid) resulted in complete eradication of all three bacteria (FIGS. 6 and 7).

### Example 3

Madin-Darby Canine Kidney Epithelial (MDCK) cells (ATCC CCL-34) were obtained from the American Type Culture Collection and maintained in Dulbecco minimal essential medium (DMEM) supplemented with 5% fetal bovine serum (FBS) and incubated at 37° C. in a humidified atmosphere with 5% CO₂ without antibiotics or antimycotic agents. MDCK cells were grown as monolayers in 75-cm² cell culture flasks. Passages between 3 and 15 were used for these experiments.

Viral strain was obtained from the laboratory stock from the British Columbia Center for Disease Control. Stocks of influenza A viruses, A/Denver/1/1957 (H1N1), were grown in MDCK for 48 hours, with medium containing 2 μg/mL modified trypsin (treated with TPCK) without serum. Stock virus was prepared as clarified cell-free supernatants. Virus concentration for stocks was determined by standard plaque assay on MDCK cells. Virus titer for this stock was 6×10⁶ plaque forming units (PFU)/mL respectively.

Aliquots of virus, diluted in phosphate buffer solution (PBS), usually 204, were spotted onto the appropriate sterile glass surface, spread into a film by means of a sterile tip, and allowed to dry, within a biosafety cabinet (normally 15-20 min). Each sample received 2 sprays (100 μL) of different concentration of NORS (0.007-0.14% w/v) at pH 3.7. Controls consisted of equivalent samples sprayed with just saline, nitrites (0.14% at pH 6) and saline at pH 3.7. After 5 minutes, all samples, and equivalent control samples were reconstituted in 1.0 mL PBS and assayed by plaque formation (plaque forming units, pfu) in the appropriate cells.

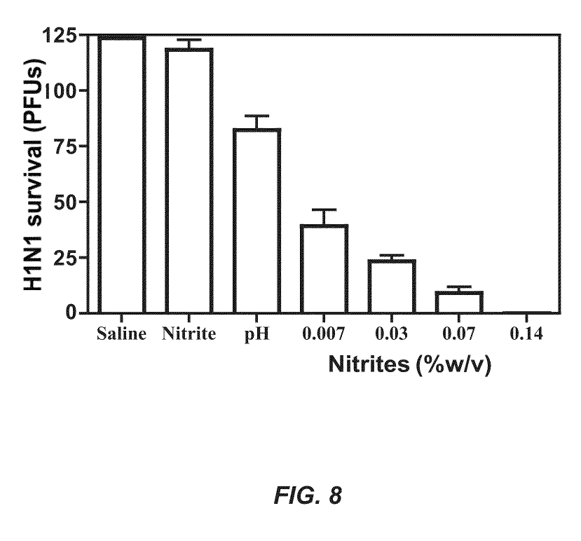

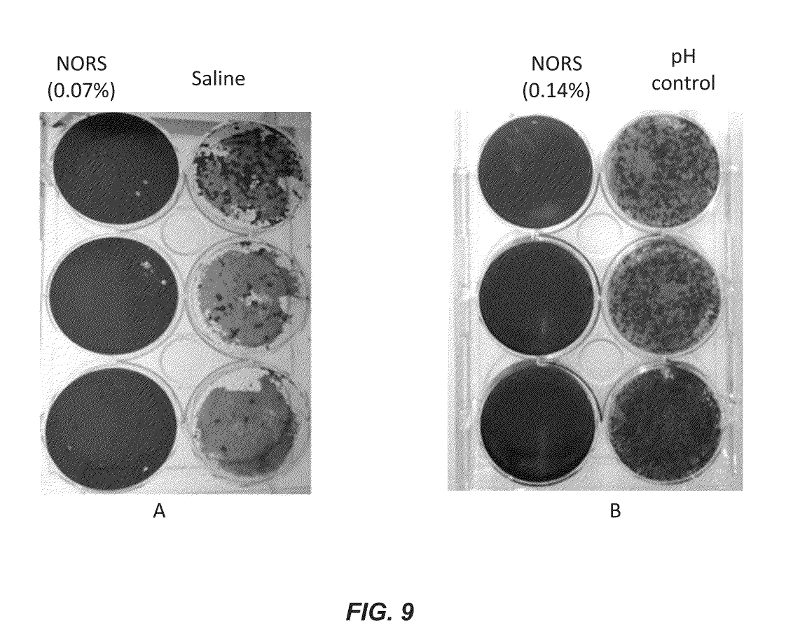

NORS comprised of citric acid and each with a different concentration of nitrites in saline solution were tested on Influenza H1N1 in order to evaluate the antiviral efficacy of the NORS. A nitrite strength of 0.07% w/v at pH 3.7 (0.08% w/v citric acid) resulted in over 90% reduction (FIGS. 8 and 9A), while 0.14% caused complete eradication of the virus (FIGS. 8 and 9B).

### Example 4

Trichophyton rubrum and Trichophyton mentagrophytes were obtained from the American Type Culture Collection (ATCC). Fungi were grown at 30° C. in Sabouraud Broth (SAB) for three days to a mycelial biomass of 1 mg/mL. Experiments on mycelial viability were done with this concentration. Conidia were isolated by shaking (on a Fisher shaker at 100 RPM) glass beads (Soda Lime 2 mm, VWR) for 60 seconds on the surface of mycelia grown on SAB agar plates for a minimum of seven days. Conidia covered glass beads were vortexed in sterile saline to suspend conidia in solution.

NORS was prepared utilizing sodium nitrite and citric acid, as previously described. Specifically, this was done by dissolving solid sodium nitrite (NaNO₂) into sterile distilled water (dH₂O) to reach a final concentration of 0.007-0.14% w/v. Then, those solutions were acidified to pH 3.7 using a predetermined mass of citric acid (up to 0.1%).

NORS containing NaNO₂ at concentrations of 0.007, 0.14, 0.35, 0.7% w/v were tested for their efficacy as antifungal agents. Sterile water (pH 6) was used as control. Sterile water adjusted to pH 3.7 using citric acid, and sterile water with 0.14% NaNO₂ (pH 6) were tested as well to determine whether either solution possessed a fungicidal effect by themselves. NORS (4 mL) was prepared and added to separate 5 mL sterile plastic tubes. 100 μl of culture containing mycelia at a biomass of 1 mg/mL was then added to each tube and incubated for 10, 20 and 30 minutes. Following incubation, samples from each tube were serially diluted and were plated on SAB agar plates. Plates were incubated at 30° C. until growth could be detected and counted (about 3 days for T. rubrum and 2 days for T. mentagrophytes). Each experiment was done in triplicate and repeated three times.

A set of control experiments were done in order to eliminate the potential antifungal effect of the citric acid concentration in the treatment solution. Different concentrations of citric acid were prepared and pH was raised to 3.7 using NaOH. The same experimental methodologies with water as a control were used to perform these tests.

The concentration of NO and other gases released from a NORS into the head space were determined by gas chromatography with a mass spectrometer detector (GC-MS). A NORS (20 mmole l⁻¹ with activator) was prepared inside the sterile 5 mL plastic tubes. Each tube was then sealed for 20 minutes after which, 1 mL of the head space above the solution was analyzed by GC-MS. GC-MS (Varian™ CP-3800 Gas Chromatograph connected to a Varian™ 1200 Quadrupole MS) analysis was performed using a standard method that had previously been created and calibrated to separate and quantify NO, NO₂ and N₂O molecules, using calibration gases. The method was set to a constant temperature of 31° C. with a sampling flow rate of 1 mL/min with helium gas as the carrier gas. Injector temperature was set to 120° C.

The concentration of NO and other gases released from the NORS into the headspace were determined by gas chromatography with a mass spectrometer detector (GC-MS). NORS (0.14% nitrites w/v) was prepared inside the sterile 5 mL plastic tubes described above. Each tube was then sealed for 30 minutes, after which 1 mL of the headspace above the solution was analyzed by GC-MS. GC-MS (Varian™ CP-3800 Gas Chromatograph connected to a Varian™ 1200 Quadrupole MS) analysis was performed using a standard method that had previously been created and calibrated to separate and quantify NO, NO₂ and N₂O molecules, using calibration gases. The method was set to a constant temperature of 31° C. with a sampling flow rate of 1 mL/min with helium gas as the carrier gas. Injector temperature was set to 120° C.

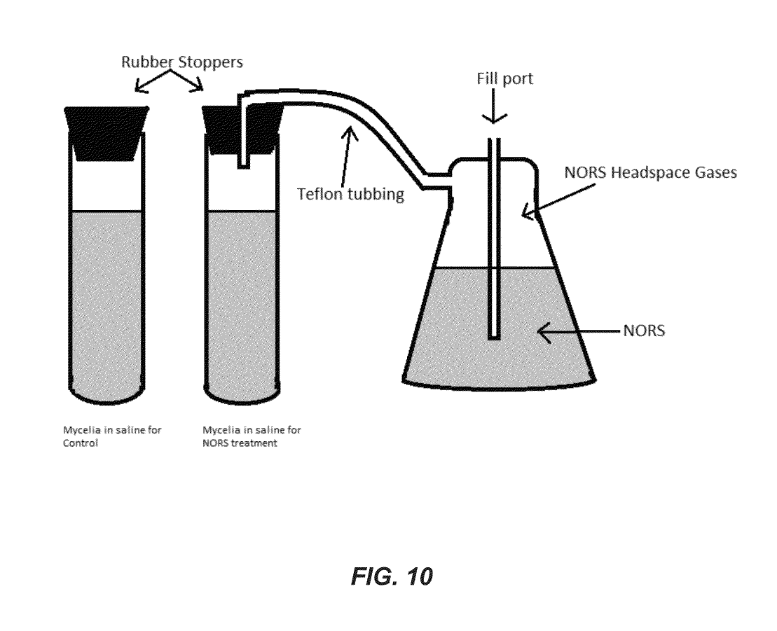

In order to demonstrate that NO, found in the headspace, is responsible for the fungicidal effect of NORS, mycelia of T. mentagrophytes (10 mL at 1 mg/mL) were combined with 20 mL of sterile saline inside a sterile glass test tube connected via Teflon tubing to a separate glass apparatus, as illustrated in FIG. 10. Sterile saline (0.9% sodium chloride) was used in replacement of sterile dH₂O in order to ensure any fungicidal activity measured was not the result of osmotic imbalances. NORS was added to the glass apparatus using a 50 mL syringe through the ‘fill port’ (FIG. 10) then sealed using paraffin laboratory film and plastic wrap. A higher strength NORS was required to produce a sufficient volume of gas to account for the much greater headspace volume in the apparatus as opposed to the 5 mL tubes previously described. The apparatus was then left at room temperature for 2, 4, 8, 16 and 24 hours (each performed separately) after which, samples from the glass test tube were plated onto SAB agar plates, incubated at 30° C. for 48 hours and fungal viability determined. The growth from the exposed test tube was compared to a control of the same contents kept alongside the exposure in a sealed glass test tube (FIG. 10). Another control study was performed with the same apparatus, using saline instead of NORS. Nitrite concentration in the attached glass test tube was measured after each time point, using Griess reagent.

T. rubrum and T. mentagrophytes were grown from conidia for a minimum of 72 hours to a mycelial biomass of 1 mg/mL. Mycelia was added to treatment or control tubes and incubated for up to 30 minutes, after which, samples were plated and concentration (cfu/mL) was determined. As NORS is formulated from nitrites and citric acid (lowering pH to 3.7), the individual exposure effect of water at pH 3.7 and 0.14% w/v nitrites at pH 6 was tested and compared to an appropriate control. Minimal to no effect was detected after a 30-minute exposure with either 0.14% sodium nitrite (pH 6) or citric acid at pH 3.7.

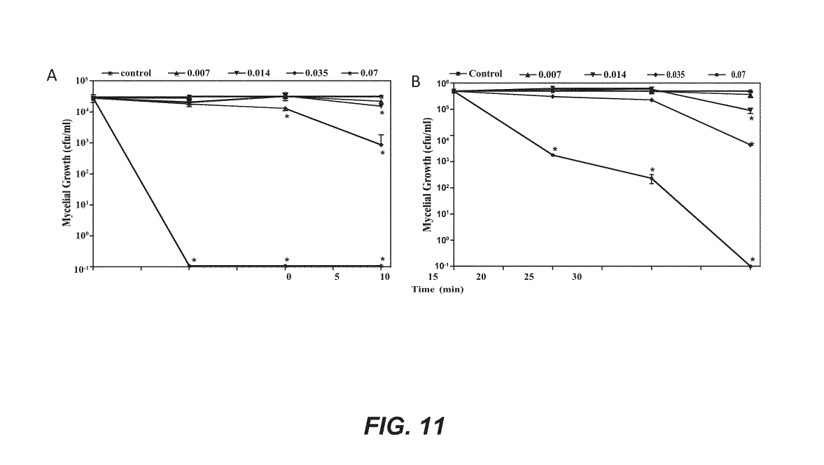

FIG. 11 shows the mycelia viability following exposure as a percentage of control. T. mentagrophytes (FIG. 11A) and T. rubrum (FIG. 11B) both demonstrated similar responses to different concentrations of NORS. Both species were tolerant to up to 0.014% w/v nitrite at pH 3.7 for up to 20 minutes demonstrating a reduction of less than 25%. While using a higher concentration of 0.035% w/v nitrite at pH 3.7 rendered a time dependent fungicidal effect starting from a significant 25% reduction after 10 minutes and reaching a 98% reduction after 30 minutes for both species. An increase to 0.7% w/v nitrite and 0.08% w/v citric acid was highly effective at eradicating mycelia resulting in a greater than 99% reduction at 10 minutes and complete kill at 30 minutes for T. mentagrophytes and a complete kill at all time points for the T. rubrum. A concentration of 0.14% w/v nitrite at pH 3.7 showed a complete kill, even after 10 minutes, for both organisms (not shown on graph). Controls with citric acid at pH 3.7, and nitrites alone, had no significant effect on mycelial growth when compared to water control.

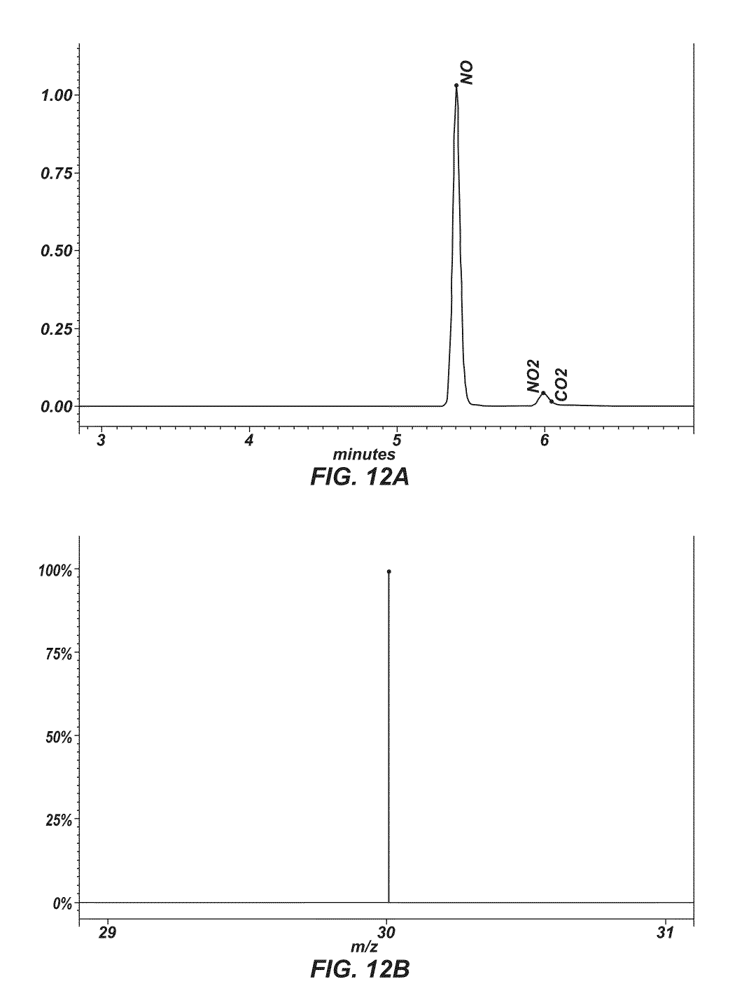

A headspace sample from the tube (containing 4 mL of 0.14% NORS) after 20 minutes was analyzed by GC-MS to determine which gaseous molecules could be detected. Specific detection was set to identify NO, NO₂, N₂O and CO₂ and their respective concentrations were determined. As revealed by the chromatogram in FIG. 12A, three types of gas molecules were detected (excluding water vapor, not shown). NO eluted at 5.4 minutes, NO₂ at 5.98 and CO₂ eluted at 6.03 minutes. No other peaks were detected in a scan program for MW 18-100. FIG. 12B shows the molecular weight of 30 for the peak at 5.4 minutes, which correlates to NO. NO concentration was found to be 170(±30) ppm; NO₂ was 40(±10) ppm. CO₂ (coming from ambient air) was found as well but not quantified. N₂O was not detected. As a comparison, headspace from a control tube had only ambient levels of CO₂ present in it.

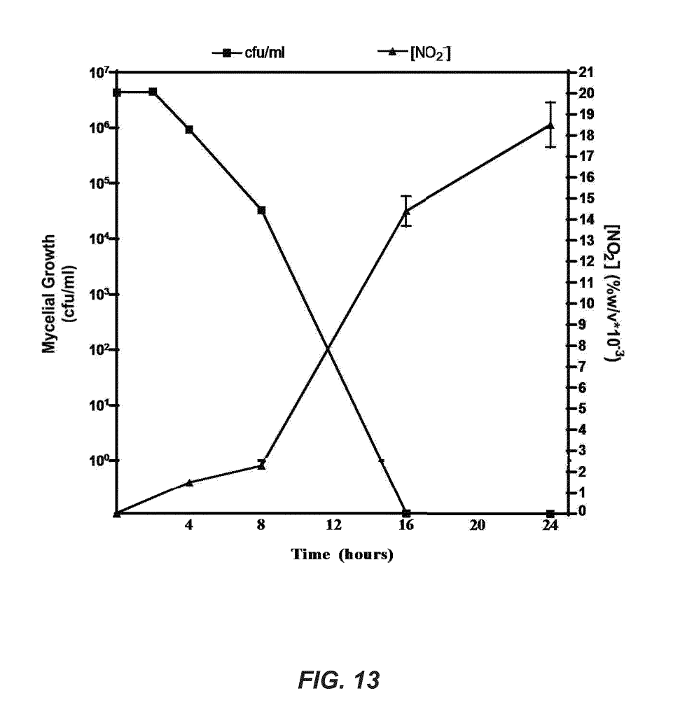

In order to demonstrate that the NO being produced by the NORS is likely the active agent responsible for the antifungal activity observed, an apparatus was constructed to ensure no direct contact occurred between fungal mycelia and the NORS, allowing only for the exchange of headspace gases (FIG. 10). The antifungal activity of the NORS headspace gases was tested on T. mentagrophytes mycelia at 1 mg/mL. Both mycelial viability and nitrite concentrations were measured after 2, 4, 8, 16, and 24 hours. FIG. 13 illustrates the antifungal activity of the NORS gases over a 24 hour period. Some antifungal effect was observed after 4 and 8 hours of exposure, where a one log₁₀ reduction in mycelial viability was observed. Complete kill resulted after 16 hours of exposure. Mycelia controls showed no significant change in concentration during these time periods. Nitrite concentrations were shown to inversely correlate with mycelial viability. Following 16 hours, where complete kill of the mycelia was reached, a nitrite concentration of 0.014% w/v was measured. Mycelia controls showed nitrite concentrations to be negligible. When using saline in the apparatus, instead of NORS, no mycelial kill was found and no gNO was found in the headspace.

### Example 5

Eighty-five, crossbred, multiple sourced, commingled commercial weaned beef calves were obtain for these studies. All studies were conducted at the Lacombe Research Centre beef research facility and all management practices followed Canadian Council of Animal Care guidelines and Canadian Beef Cattle Code of Practice guidelines. In addition, the research protocols were reviewed and approved by the Lacombe Research Centre animal care committee. The calves were procured through a conventional auction system and all animals had been exposed to between 4-6 h of transport prior to the study. These calves were chosen in order to provide study groups displaying a bovine respiratory disease (BRDc) incidence range of 30-60% which is typical of the beef industry in Canada for these “put together” herds of cattle. On arrival at Lacombe the calves were off loaded, weighed, and sampled for saliva and blood using methods known in the art.

The calves were randomized to treatment and control groups, labeled with color coded ear tags and numbers. NORS was delivered with a spray device. This solution was tested and verified to release 160 ppm NO in a 3 l/m flow of medical air (Praxair, Canada), for 30 min. In brief, 32 ml of the solution was sprayed into a two inch diameter vinyl chloride tube and inserted into environmentally controlled system where NO was measured using chemiluminescence (Sievers Nitric Oxide Analyzer NOA 280i). Animals were restrained in a conventional hydraulic cattle-handling catch and given either a placebo (saline) or treatment (NO) by an individual blinded as to the intervention. Each animal received 1 spray (8 ml), alternating into each nostril, twice, for a total of 32 ml before being released into the feeding lot pen areas. The duration of treatment administration was less than 5 s.

Animals were then placed into outdoor pens measuring approximately 60×60 m and were bunk feed ad libitum a balanced cereal silage diet, which met or exceeded National Research Council recommendations. The animals also had free access to water and were provided a straw bedding area with a roof covering.

While contained in their receiving pens the calves were monitored daily by trained personnel, whom were blinded as to the treatment interventions, for clinical signs of illness. Briefly, clinical scores were designed to identify BRDc and were based on the appearance of four criteria as follows:

Respiratory insult: (0-5): 0=no insult, normal breath sounds (NBS); 1=Very Fine Crackle (rale) (VFCR) on auscultation and/or a moderate cough; 2=Fine Crackle (subcrepitant) (FCR) on auscultation and/or a moderate nasal discharge and moderate cough; 3=Medium Crackle (crepitant) (MCR) on auscultation and/or a moderate to severe viscous nasal discharge with cough; 4=Course Crackles (CCR), tachypnoea (>15% of the norm) and/or a severe nasal discharge with respiratory distress and obtunded lung sounds and 5=CCR with dyspnoea, tachypnoea, marked respiratory distress and/or lung consolidation.

Digestive insult: (0-5): 0=no insult, normal, eating and drinking; 1=mild or slight diarrhoea with slight dehydration (<5%) and reduced eating; 2=moderate diarrhoea with 10% dehydration and reduced feed intake (<50%); 3=moderate to severe diarrhoea with 10% or less of feed intake and more than 10% dehydration; 4=severe diarrhoea, and less than 10% of normal feed intake and 5=severe diarrhea and not eating, not drinking and dehydrated.

Temperature score: Core temperature (rectal) (0-5): 0=<37.7° C.; 1=37.7-38.2° C.; 2=38.3-38.8° C.; 3=38.9-39.4° C.; 4=39.5-40.0° C. and 5=>40° C. Rectal or core temperatures for the calves were collected at the start and end of the study only as these were the times that the animals were restrained.

Disposition or lethargy score: (0-5): 0=no lethargy, normal posture; 1=mild anorexia or listlessness, depressed appearance; 2=moderate lethargy and depression, slow to rise, anorectic; 3=recumbent or abnormal posture, largely depressed; 4=prostrate, recumbent or abnormal posture and 5=death.

Animals displaying overt clinical symptoms of BRDc as identified by a blinded pen keeper were rescued and subsequently received immediate treatment as recommended by the Lacombe Research Centre veterinarian followed by continued monitoring and re-treatment if required. These animals were classified as true positive (TP) in the statistical analysis.

The determination of an animal true positive or negative for BRDc was based on the comparison to a set of “gold standard” values as known in the art. This approach is commonly promoted in both veterinary and human medical diagnostic studies. In the current study, the criteria for a true positive animal for BRDc was defined as an animal displaying three or more of the following signs; a core temperature of >40° C. (or 103.5° F.), a white blood cell count of less than 7 or greater than 11×1000/1 L, a clinical score of >3 or a neutrophil/lymphocyte ratio of <0.1 (leucopaenia) or >0.8 (neutrophilia). A true negative animal was defined as an animal displaying a score of 0 or 1.

Salivary and serum cortisol levels were analyzed using an enzymatic assay known in the art. Hematology values were measured on a Cell-Dyn 700 Hematology Analyser (Sequoia—Turner Corp. Mountain View, Calif.). Differential blood cell counts were determined utilizing stained blood smears (Geisma-Wright quick stain) and direct microscope examination of 100 cells. For laboratory assessments, all calves were monitored at the beginning of the study and again three to four weeks later.

The results were analyzed using the unpaired Student's t-test for comparison between any two groups. Group means were statistically tested by least squares means (two-tailed t-test). Data analysis and graphical presentation were done using a commercial statistics package (Graphpad-Prism V 3.0, GraphPad Software Inc., USA). Unless otherwise specified, p<0.05 indicated statistical significance. Results were reported as the mean±standard deviation.

Three different studies were done. Eighty-six multi-sourced, commingled commercial weaned beef calves were enrolled in the study and randomized into either treatment or control cohort. When analyzing the results, animals that arrived to the feedlot as TP, were discarded from the analysis which left 40 control animals and 42 in the treatment group. As can be seen in Table 2, the remaining animal cohorts were not significantly different in any of the parameters tested. No significant difference was found between average weight (p=0.81) of the two groups with values of 287.7 kg (SD 37.8) for control and 290.9 kg (SD 46.8) for treatment. All baseline blood work including total white blood cells and specifically neutrophils, lymphocytes, monocytes, eosinophils and basophils were not significantly different between the two cohorts. Three animals in the control group and none in the treatment group were identified by the pen keeper using normal commercial criteria and were rescued with conventional antibiotics and categorized as treatment failures for statistical analysis.

TABLE 2
Demographics - the average value for treatment or control groups for weight, temperature and all blood parameters that were tested.

| | Weight | Temp F. | Wbc | Neut | % Neut | Lymph | % Lymph | Mono | % Mono | Eos | % Eos | Baso | % Baso | Rbc | Hgb | Hctm |
|---|---|---|---|---|---|---|---|---|---|---|---|---|---|---|---|---|
| C avg | 633 | 103.0 | 8.3 | 1.1 | 13.6 | 5.4 | 65.1 | 0.8 | 10.5 | 1.0 | 10.1 | 0.1 | 0.7 | 9.8 | 13.3 | 39.1 |
| C std | 83 | 1.1 | 1.9 | 0.9 | 9.3 | 1.7 | 12.5 | 0.3 | 4.3 | 0.9 | 7.4 | 0.0 | 0.3 | 1.2 | 1.2 | 3.2 |
| Tx avg | 640 | 102.9 | 8.3 | 1.1 | 13.7 | 5.4 | 64.5 | 0.9 | 11.0 | 0.8 | 9.7 | 0.1 | 1.1 | 9.6 | 13.2 | 38.5 |
| Tx std | 104 | 0.9 | 1.6 | 0.9 | 10.3 | 1.7 | 14.4 | 0.5 | 6.5 | 0.7 | 8.1 | 0.1 | 2.2 | 1.2 | 1.3 | 3.8 |
| T test | 0.8 | 0.6 | 1.0 | 1.0 | 1.0 | 1.0 | 0.8 | 0.6 | 0.7 | 0.4 | 0.8 | 0.3 | 0.3 | 0.5 | 0.7 | 0.5 |

All animals tolerated the nitric oxide treatments well. Some of the animals sneezed but none exhibited coughing or other clinical signs of distress. However, behavioral differences in the tolerance of treatment between the cohorts were not quantified. There were no adverse events nor serious adverse events observed in either cohort. No animals died during the time of the study. Mean salivary and cortisol levels were equivalent in each group (Control 5.4±5.7 nmol/L; Treatment 6.66±5.5 nmol/L) without significant differences (p=0.09).

As can be seen in Table 3, during days 1-14, 13 animals from the control group and 5 animals from the treatment group were identified as TP. The table shows values recorded for all 4 parameters determining TP/TN for all TP animals. Temperature, clinical score, white blood count, neutrophil/lymphocyte ratio were also included. All sick animals had 3 or 4 parameters recorded below or above the defining value for TP. This scoring approach provides a more robust definition of sick animals as compared to looking at just a temperature threshold alone. All animals had clinical scores above 3 and 15 out the 18 animals had temperature recorded as 103.5° F. or higher. Thirteen out of the 18 TP animals were also recognized by the pen keeper as sick.

TABLE 3
Values recorded for all 4 parameters determining TP/TN for all sick
animals in both groups during the first 2 weeks after arrival to feedlot (TP
indicators are highlighted).

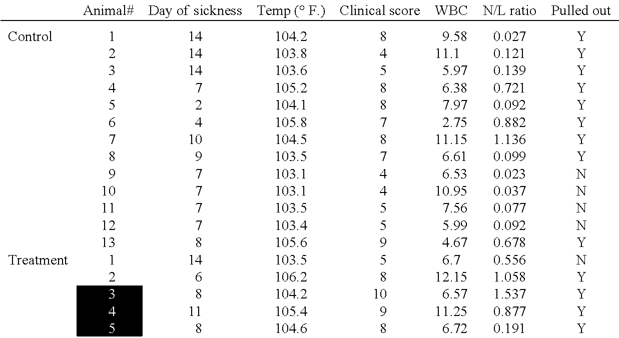

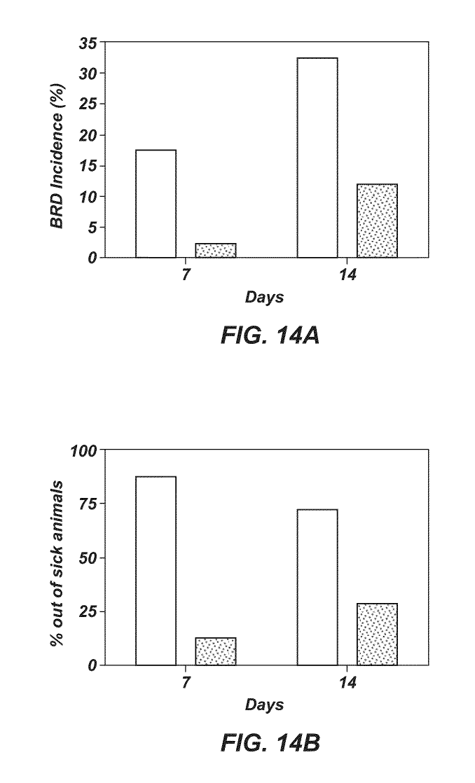

In terms of a BRDc incidence in this model, of these 82 calves evaluated, after 7 days post arrival, 8 displayed true positive for BRDc (10%). As shown in FIG. 14a, 7 animals (17.5%) out of the 40 in the control group and 1 (2.4%) out of 42 in the NO treated group were identified as TP in the first week. Another way to look at these data (FIG. 14b) is that of these 8 animals, one (12.5%) was from the NO treated group and seven (87.5%) were from the saline control group. This represents a very significant reduction of the incidence of BRDc between the treatment and control cohorts with a single NORS treatment upon arrival into the stockyard (p<0.001). During the first 14 days, 18 animals (22%) had an incidence of BRDc and of these 13 (72.2%) were in the control group whereas only 5 (27.8%) were in the treatment cohort (FIG. 14b).

TABLE 4
Day of sickness. Table shows the day in which an animal
was recorded sick, post arrival to feedlot.

| | | Day | Day | Day | Day | Day |
|---|---|---|---|---|---|---|
| | | 0 | 1-7 | 8-10 | 11-14 | 15-28 |
| Con- | # New TP | 3 | 7 | 3 | 3 | 0 |
| trol | Cumulative sick (from day 1) | | 7 | 10 | 13 | 13 |
| | Remaining | 40 | 33 | 30 | 27 | 27 |
| Tx | # New TP | 2 | 1 | 2 | 2 | 6 |
| | Cumulative sick (from day 1) | | 1 | 3 | 5 | 11 |
| | Remaining | 42 | 41 | 39 | 37 | 31 |

Table 4 shows new sick animals per time period, defined at either testing day or when animals were pulled out. Animals were pulled out of the study and deemed clinically sick when the animal herdsman determined that the animal was deemed sick by normal commercial feedlot assessment. Looking at the first 2 weeks after treatment, 13 (32.5% of total control group) animals out of the control group had a TP score, while only 5 (11.9% of total Tx group) had a TP score out of the treatment group. It should be noted as well that 3 more animals out of the control group (and none of the treatment) were pulled out during these 2 weeks although they were borderline and did not turn out to be TP. These animals as having a TP incidence of BRDc in the above analysis were included in the results. The median day that an animal became sick, after arriving at the feedlot, was 8 days in the control compared to 18 days in the treatment group. When looking at 15-28 days post treatment, there was no effect seen. Further, when looking at the cumulative number of sick animals from day 1 to day 28, 33% of the control group and 26% of the treatment group were identified as TP.

These data, collected from three separate randomized and blinded studies performed in a conventional feedlot, show that NO significantly decreased the incidence of BRDc, as defined by true positive rigor, by a difference of 75% as compared to a saline placebo (87.5% of sick animals were from control vs 12.5% from treatment group). This test used a naturally occurring BRDc model from multi-sourced co-mingled animals acquired from a commercial auction and transferred to a research feedlot. Further, duration of effectiveness for NORS treatment was up to 14 days which is similar to most antibiotics.

Additionally, once an animal was treated with NO, if it did become sick, the illness was delayed. The average day of an animal from treatment group to get sick was 18 days post arrival to the research feedlot while it was 8 days for the control group. Reduction and delayed onset of BRDc observed in this study is likely to be related to nitric oxide released from NORS. Release of NO from NORS was verified, prior to the study, in a bench test model where a continuous flow of air over NORS resulted in 160 ppm nitric oxide.

These results are similar to those reported in metaphylactic use of antibiotics to treat BRDc in clinical trials. Metaphylactic use of antibiotics has been shown to reduce and delay the incidence of BRDc as defined by an undifferentiated fever of greater than 104° F. (41.7° C.) in beef cattle entering feedlots. Cattle presenting with undifferentiated fever treated with metaphylactic antibiotics have lower incidence of mortality, a higher weight and quality of meat when they are dressed.

Unlike antibiotics, requiring pre-slaughter withdrawal periods (as determined by the FDA), nitric oxide is unlikely to have any residue in the meat product, due to its short half-life. Moreover, antibiotics and NO have different mechanisms of actions; antibiotics are classified based on specific targets whereas gNO possesses a wide-ranging antimicrobial targets that are essential to the basic biochemistry of the microbes. Recent studies in bacteria have suggested that NO has an affinity for reduced surface thiols and divalent metal centres in intracellular enzymes. It is predicted that nitric oxide will attach to surface cysteines causing the formation of S-nitrosylation (-SNO) sites, which perturb enzyme structure and/or catalytic activity. Another mechanism is the reaction of NO with oxygen or superoxide to spontaneously produce reactive nitrogen and oxygen intermediates, resulting in the formation of multiple antimicrobial intermediates. These reactive nitrogen oxide species cause oxidative and nitrosative damage by altering DNA, inhibiting enzyme function, and inducing lipid peroxidation, which account for the majority of NO cytotoxic effects. As a result, nitric oxide seems to be effective against a wide spectrum of bacteria, viruses and fungi while antibiotics are specific to bacteria. Therefore, the potential added advantage of NO, over antibiotics, is its anti-viral effect. NO may ameliorate the pathogenesis of BRDc by lowering the viral load and thereby reducing susceptibility of the animal to bacterial infection.

A major concern in food producing animals is the emergence of drug resistant bacteria. Nitric oxide production and use as the first line of defense in the immune system has been preserved genetically across many species. Since NO is a broad non-specific antimicrobial, rendered by its multiple intracellular biochemical targets, the risk of developing resistance to NO is likely to be ameliorated. It is postulated that the microbicidal activity of the NO released from NORS is fundamental to the biochemistry of both bacteria and viruses that survivors are unlikely to induce microbes to become “drug resistant”. This is evidenced by the lack of reported resistant bacteria/viruses in the human infant population that has been receiving suboptimal “antimicrobial” doses over the last decade.

The foregoing serves as evidence that NO treatment is well tolerated and does not result in undue stress of the animal as measured by salivary and serum cortisol levels. No adverse events were observed.

### Example 6

This study was conducted at a commercially registered feedlot facility in Western Canada (Westwold, British Columbia). All management practices followed the Canadian Council of Animal Care guidelines and Canadian Beef Cattle Code of Practice guidelines. In addition, the research protocols adhered to the Experimental Study Certificate approved by the Health Canada Veterinary Drug Directorate and the Thompson Rivers University animal care committee. Thirteen, crossbred, multiple-sourced, commingled commercial weaned beef calves were procured through a conventional auction system. All animals were exposed to approximately 4-6 h of transport prior to the study. These calves were chosen in order to provide study groups displaying a BRDc incidence range of 30-60% which is typical of the beef industry in Canada for this type of a cattle population. On arrival at the feedlot the calves were off loaded, randomized into one of three cohorts, received ear tags, were vaccinated (Bovi-Shield® GOLD FP™ 5; Pfizer, INFORCE™ 3; Pfizer, M. Haemolytica Bacterin-Toxoid; Pfizer) and weighed.

Calves consisted of 3 groups as follows: (1) Control group—received saline as placebo (n=4), (2) Treatment group—2 sprays of NO treatment in each nostril—32 ml in total (n=5) and (3) Treatment group with 5 times the normal NO treatment dose of 160 ml in total in each treatment. All groups were treated with NO on arrival approximately 2 min after giving the vaccines. Animals were then placed into 2 outdoor corrals, separated into control or treatment groups. They were fed chopped hay, grain screening pellets, along with alfalfa/grass and barley silage to provide a complete ration which met or exceeded National Research Council recommendations. The animals also had free access to water and were provided with sawdust bedding.

While contained in their receiving pens the calves were monitored daily by trained personnel for clinical signs of illness. Animals displaying overt clinical symptoms of BRDc upon initial screening received immediate antibiotic treatment as recommended by the owner, followed by continued monitoring and retreatment if required. These animals were excluded from the study analysis. Animals that were enrolled into the study and subsequently identified as being sick, both at the weekly handling time or during daily monitoring, were rescued and received an antibiotic (EXCENEL® RTU). These animals were categorized as treatment failures.

The breed, weight, and rectal temperature were recorded on arrival at the feedlot. Temperatures were measured by three different methods for use in another study on disease detection (orbital infrared thermography of the eye, rectal temperature and by reticulum bolus). Reticulum bolus temperatures were used for analysis in the study as they were found to be the most accurate and reliable measure. Conventional rectal temperature was initially taken upon screening and then taken once a week during study interventions. Reticulum bolus (Bella Bolus, Bella Ag LLC, CO) temperature measurements were obtained using a system that incorporated a bolus, receiver, base station and computer Infrared orbital eye temperatures were taken at the squeeze by a certified infrared thermographer. The bolus was administered orally using a standard balling gun and permanently resided in the reticulum of the animal throughout the entire duration of the study. Data was collected utilizing a micro radio transmitter to send out temperatures and identification readings from each of the animals. After insertion down the animal's throat, the bolus monitored the reticulum temperature and transmitted it every 5-6 min to the receiver at 300-450 MHz. The receiver then retransmitted the data to the base station at 2.4 GHz where an attached dedicated laptop computer logged it to a database. The receiving station containing the receiver and computer was located between the two pens.

A nitric oxide releasing solution (NORS) was supplied in a 5 L spray device, which contained 2 L of the NORS. The solution was prepared on site just prior to administration. This solution consisted of a nitrite strength of 60 mM which was previously tested to release 160 ppm NO in a 3 L/min flow of gas as verified by chemiluminescence analysis (280i, General Electric, CO). Animals were briefly restrained in a conventional hydraulic cattle handling squeeze and given either saline or NORS by a trained research assistant. Each animal in the control and normal treatment dosing groups received 1 spray (8 ml), alternating into each nostril, for a total of 32 ml of either of the interventions before being released into the feeding lot pen areas. Each animal in the second dosing group received 5 times the above-described dosing volume, for a total of 160 ml. Animals received these treatments weekly for four consecutive weeks.

Blood samples were collected on day 14. Blood was collected by a licensed veterinarian via jugular venipuncture before treatment, 5 min post treatment and 30 min post treatment interventions. Each sample was placed in one of 3 appropriately prepared collection tubes—one for each measurement: Cortisol, methemoglobin percent (MetHg) and nitrites. Serum cortisol was analyzed by Kamloops Large Animal Veterinary Clinic LTD. (1465 Cariboo Place, Kamloops, BC V2C 5Z3). All blood samples were transferred to Thompson River University (TRU) on ice for measurements of MetHg. Blood gas analysis was done for co-oximetric measurement of MetHg using an ABL 800 FLEX analyzer (Radiometer America Inc., OH, USA). Blood gases including arterial oxygen, carbon dioxide, pH, bicarbonate and electrolytes were also measured at that time.

Fractional exhaled concentration of NO (FENO) was measured using a chemiluminescence analyzer (280i, GE, CO). A FENO baseline measurement was obtained for each subject by recording for 1 min before and after treatment intervention until FENO levels returned back to baseline. The sampling tube had a water filter to prevent liquid from getting into the device. The filter was at the distal end and was held as close as possible to the animal's nostril. The same person did all of the animal handling to reduce handler variation. The machine was calibrated before each use with standard calibration gases as per manufacturer's instructions.

On day 21, all 13 animals were monitored to examine the behavioral response to each of the different treatment types. Monitoring was achieved by utilizing a 1 min video recording, collected immediately after administration of the treatment intervention. MPEG video recordings were given to an experienced animal ethnologist in the Biological Sciences department of Thompson Rivers University to score who was blinded to each of the treatment types. To determine the behavioral response to treatment administration, 6 variables were scored: blinks, nods, head movements, head jerks, vocalizations and overall post-treatment response. Each treatment administration response was scored on a range of 0-2, with 0 being no observed response to the treatment, 1 being movement of the head when the applicator was placed in the nostril, and 2 being strong jerks or movement away from the applicator. After administration of the treatment the number of blinks and vocalizations were recorded during the observation period. Nods and head movements were recorded when the subject moved it's head more than approximately 10° in any direction. Head jerks were recorded when the subject moved rapidly, generally appearing to attempt to pull its head out of the restraint or rearing upwards. Finally, an overall post-treatment response was scored on a scale of 0-4 (0=no response, 1=slight response, 2=response, potentially some agitation, 3=response, clearly agitated, 4=strong response, highly agitated, aggressive, upset).

On slaughter day, 7 days post final treatment, the respiratory tracts as well as the heart, liver, spleen and kidneys from twelve feedlot calves were examined by a board certified veterinary pathologists during slaughter at a Canadian Food Inspection Agency inspected commercial packer, in Chilliwack, British Columbia. Each respiratory tract examined consisted of the larynx and adjacent pharyngeal tissue, trachea, and lung. Following gross examination and prior to collecting tissue samples for histology, photographs were taken of each set of lungs and the ear tag from each animal. The entire heart, liver, spleen and both kidneys of all animals were also examined, but were not photographed.

Samples of trachea and the anterior, middle and caudal lobes from both lungs of each animal were collected, fixed in 10% neutral buffered formalin and held to process for histopathologic examination. Samples of lung and each of the other organs listed above from each animal were collected, frozen, and forwarded to the Division of Respiratory Medicine at the University of British Columbia for further nitrite residue analysis.

Following fixation, the trachea and lung samples were forwarded for tissue processing to the Advanced Microscopy Laboratory, Department of Biological Sciences, University of Alberta in Edmonton, Alberta, Canada. Each of the fixed tissues was reexamined by a veterinary pathologist who then sectioned them and placed the selected sections into tissue cassettes. Samples were then processed into paraffin blocks, sectioned at 5 lm, stained with hematoxyin and eosin and cover slipped using standard histologic techniques. Following preparation, microscope slides were examined by the pathologist, who was blind to the study group assignment of each animal. All slides were found to be well stained and representative of either the trachea or lobe of the lung from which they were taken, and therefore were suitable for comparison to similar sections for the same organ from the other animals in the study.

For gross changes, in order to determine if there were any differences between the three groups, scores were assigned to the amount of consolidation observed according to the following key:

0=Normal lobe.

1=Some consolidation of individual lobules within a lobe.

2=Confluent consolidation of lobules within a lobe, less than 50% affected.

3=More than 50% of the tissue in a lobe consolidated.

In addition, scores were assigned for the degree of pleuritis as follows:

0=No pleuritis.

1=Scattered fibrin exudate on pleural surfaces of one lobe.

2=Thin sheets of fibrin on pleural surfaces of one or more lobes.

3=Mass of fibrin greater than 1 cm thick, fresh or undergoing organization on more one or more lobes.

A separate scoring system for the microscopic changes was devised for each of the trachea and the lungs as follows (Pleural adhesions were not included in the scoring system as they are secondary events):

Tracheal Scoring:

0=Microscopically normal.

1=Effacement of brush border or submucosal mononuclear cell infiltrate or acute neutrophilic inflammation.

Lung Scoring:

0=Microscopically normal section.

1=Bronchial cellular reaction, no parenchymal involvement.

2=Bronchitis/bronchiolitis with parenchymal involvement.

A multiplier based on the microscopic assessment of mild (1), moderate (2) and severe (3) was then applied to each score assigned. As with the gross findings, the resultant scores for each individual animal in the group were added to give a group score.

All frozen organs collected at slaughter day, as well as meat samples, were tested for nitrite residue. Organs/tissues included: kidney, liver, spleen, fat and meat. Two grams from each tissue section was placed in a tube and homogenized with phosphate buffer solution (PBS). Nitrites were then extracted with Ethanol.

Blood samples for nitrite analysis were collected on day 14 (as mentioned above). All samples for nitrite measurement were placed in heparinized tubes and centrifuged for 5 min at 5000 RPM. The supernatant was recovered and placed in Eppendorff tubes, placed on dry ice, and then samples were immediately transferred to a −80° C. freezer until processing. Nitrite measurements were performed using a chemiluminescent liquid interface technique according to the manufacturer's instructions (280i, General Electric, CO).

Data in all the above exposure experiments were expressed as mean standard deviation (S.D.). Statistical analysis of data obtained in all experiments, were performed using a one-way analysis of variance (ANOVA) and Tukey's Multiple Comparison Test. A value of P<0.05 was considered statistically significant. Data analysis and graphical presentation were done using a commercial statistics package (Graphpad-Prism V 3.0, GraphPad Software Inc., USA).

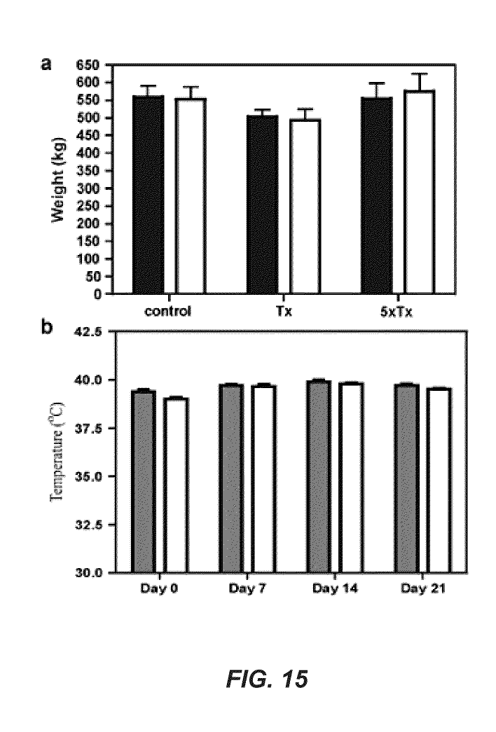

As can be seen in FIG. 15a, there were no significant differences (P=0.05) between average weights of the cohorts upon arrival to the feedlot versus at day of slaughter for the 12 animals sent to the packing plant. No significant difference in rectal temperature was found, during the 4 weeks, between the control (4 animals) and the treatment group (5 animals)—FIG. 15b.

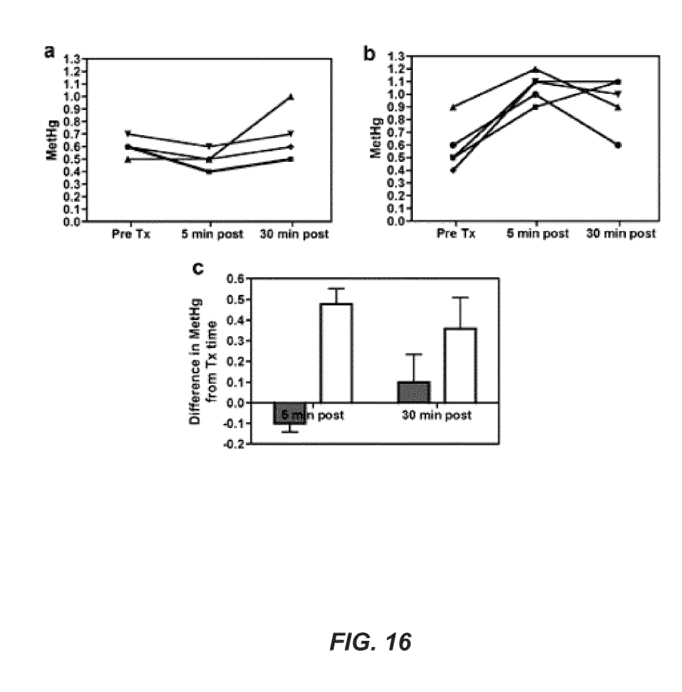

All three parameters measured for bioavailability (MetHg, FENO and nitrites in serum) showed biochemical changes within 5 min post treatment. MetHg was measured using Cooximetry on blood samples taken before treatment and at 5 and 30 min post treatment with either NORS or saline control. The saline control group (FIG. 16a) did not have any significant difference between MetHg values before and after treatment. On the other hand, the NORS treatment group had higher values of MetHg 5 and 30 min after administering the treatment (FIG. 16b). FIG. 16 shows the values of MetHg for the control and treatment group animals (FIGS. 16a and 16b), and the average difference between the MetHg value at 5 and 30 min post treatment, compared to baseline (FIG. 16c). There was a significant difference observed at the 5 min post-treatment time between the NORS and the saline control group. The MetHg value was, on average, 4.8 points higher in the treatment group compared to 0.1 lower in the control group. Small but insignificant differences were found after 30 min between treatments, although for the NORS treatment group, values stayed significantly higher than the baseline measure.

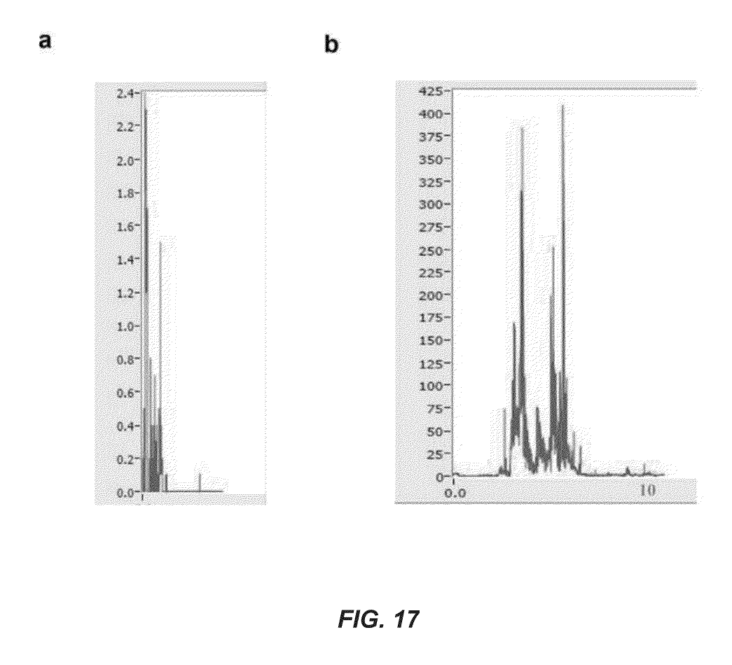

As can be seen in FIG. 17, when administering NORS to the animal, the FENO were high enough to be detected by chemiluminescent analysis within seconds to minutes following the NORS treatment. FIG. 17a shows the FENO measured after giving saline to the animal (2.4 ppb) while the FIG. 17b shows FENO after giving NORS (around 400 ppb for approximately 5 min). This was measured outside the nostril, while diluted with air and thus, levels are much lower than actual FENO levels, but it still shows the NO gas is present compared to the saline control.

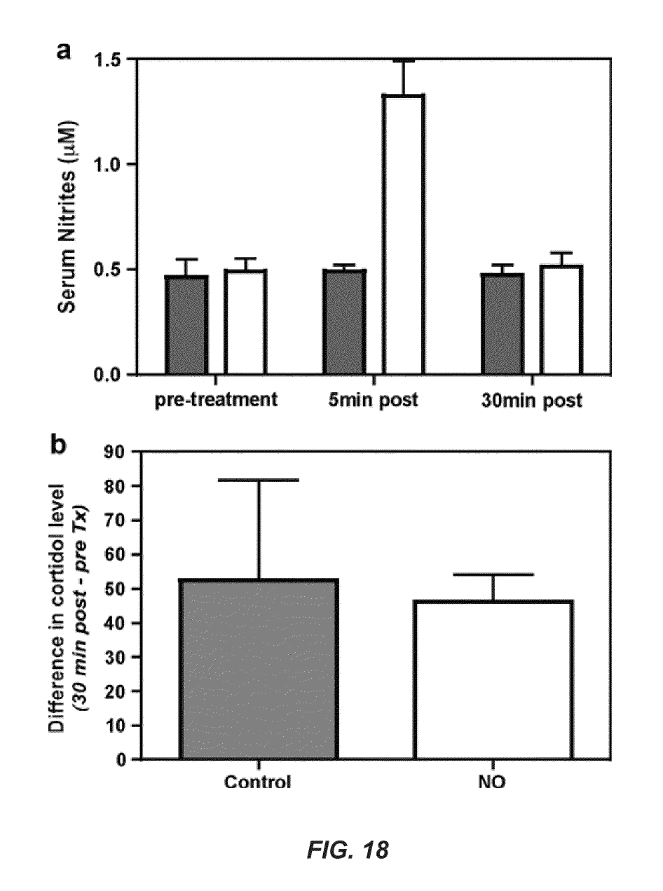

Nitrites were measured using the chemiluminescence liquid interface technique. Samples were extracted with cold ethanol and 50 μl was injected. As seen in FIG. 18a, 5 min post treatment there was a raise in the nitrite concentration in the animal's serum. By 30 min post treatment there was no significant difference (P>0.05) from the control group.

For toxicology purposes, another group of animals that received 5 times the original dose was tested as well. In general, treatment was well tolerated and no residues were found. The pathology did not show any significant (P<0.05) difference from the control.

Blood cortisol levels were measured as an indicator of increased stress level during and after administration of treatment interventions. Animals, in general, had slightly higher but not significant (P>0.1) cortisol levels at 30 min compared to pre-treatment levels (both control and treatment group). As seen in FIG. 18b, there was no significant difference in the average cortisol change. A change was observed in cortisol levels 30 min post treatment, compared to the levels measured prior to treatment.

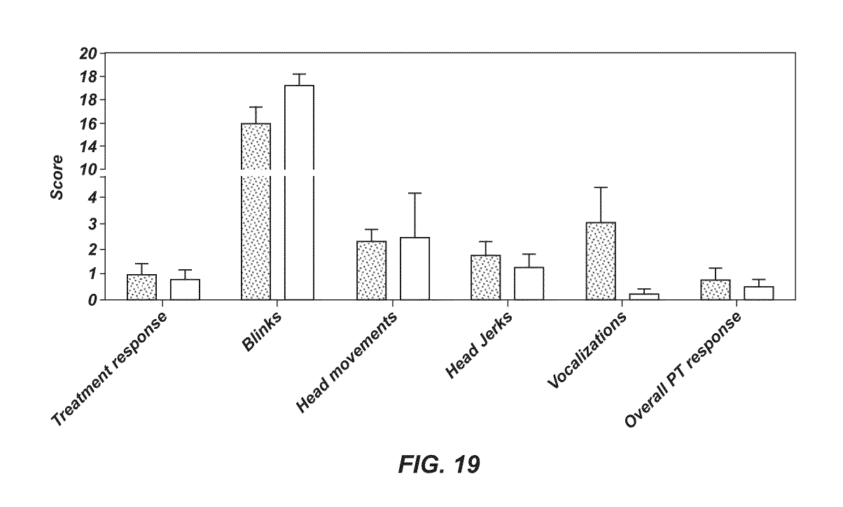

Six parameters were observed to monitor potential changes in behavior as a result of treatment. In five out of the six animals, there was no significant difference between the control and the NORS treatment group scores (FIG. 19). Increased vocalization was observed in the control group compared to the treatment group. This was determined to be unrelated to the treatment regime and likely attributable to difference between the breed types.

The gross lesions observed in each animal are listed in Table 5. Lesions were typical of those observed in feedlot calves in Western Canada in the fall. They consisted of bronchitis, bronchopneumonia and pleural fibrosis. All lesions were in various stages of resolution, indicating that there had been an outbreak of respiratory disease in the feedlot in the recent past, and that the affected calves in this cohort were all recovering.

TABLE 5
The gross lesions observed in each calf.

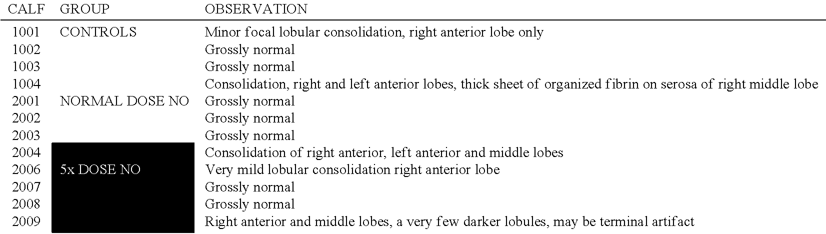

There are six main lobes in the bovine lung (assuming the right middle and accessory lobes are one). Therefore, each group of four animals has a total of 24 lung lobes. Using the key to assign a score to each group, no difference was observed between treatment and control score (9 and 10 respectively).

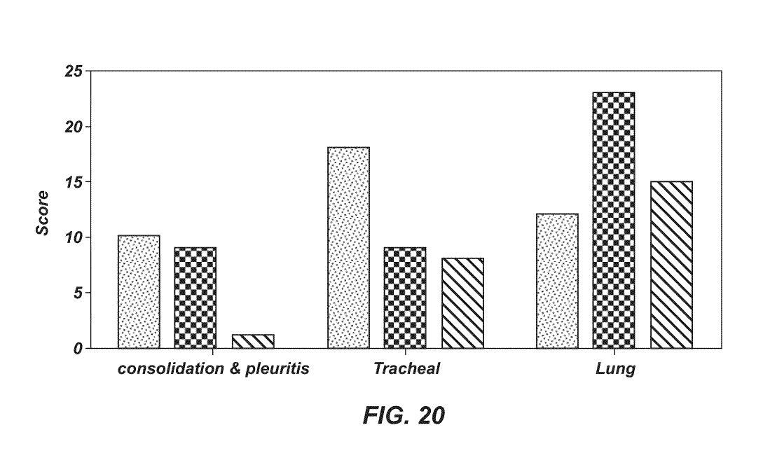

Histopathologic findings for each animal are presented in FIG. 20. All animals had some infiltration of the tracheal submucosa with mononuclear inflammatory cells while sections of lung lobes varied from entirely normal to complete consolidation. In general there was no histopathologic effect seen as a result of the treatment (FIG. 20). The group scores assigned for gross observations are essentially the same for the normal dose group than for the controls, but much lower for the 5× dose group than control.

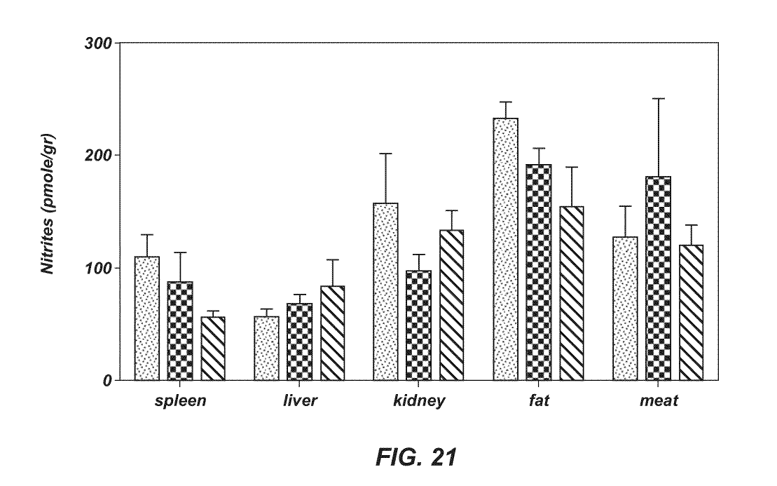

As can be seen in FIG. 21, there was no significant difference found between the control and treatment nitrite content in all organs' sampled.

These studies demonstrate that treatment with NORS is well tolerated and did not result in undue stress on the calves. There was no difference between treatment cortisol levels and control groups, which implies no extra stress on the animals due to the NORS treatment. There were no behavior changes either, due to the treatment. There is consistent support in the literature for the link between cortisol and anxiety-related behavior in beef cattle, especially when cattle are restrained in a squeeze chute. When cattle are stressed, which is often paralleled by observable changes in behavior, cortisol levels are generally expected to elevate. As there were no differences observed in either cortisol levels or in the observed behavioral measures between treatments, it can reasonably be concluded that the NORS treatment caused no additional stress to the cattle over and above that which was experienced during application of the control treatment. This is important, as stress is known to exacerbate and increase the incidence of BRDc.

Safety of NORS treatment and lack of toxicological residue is clear from both the gross and microscopic pathology findings. There were no adverse anatomic effects of the NORS treatment, even when 5 times the dose was used. Interestingly, the tracheal scores for both treatment groups were half those of the control group. It is important to note that these numbers have not been subjected to statistical analysis. It is possible that the effect of NO might be expected to decrease with distance from the nasal cavity, and perhaps this finding might indicate a beneficial effect of NO on reducing inflammation in the upper respiratory tract. Metabolite residues from NO were not found in any major organs or in the meat itself. Together, these results suggest that NORS treatment during feedlot arrival is safe and well tolerated by the animals.

Additionally, NORS resulted in NO bioavailability that was confirmed by the rise of F_{E}NO in the treated animal and by the expected transient rise in MetHg percent, indicating that NO was available within the respiratory tract and metabolized in the serum into increased nitrite levels. F_{E}NO is often used to measure changes of endogenous NO production as an inflammatory marker in diseases such as asthma (low parts per billion (ppb) levels) or to monitor NO therapy treatments for neonates with persistent pulmonary hypertension (high ppb levels). The significant rise in F_{E}NO to high ppb levels after treatment with the NORS nasal spray shows that the solution was producing NO and that animals were indeed treated with gNO.

The corresponding rise in MetHg percentage confirmed that there was sufficient NO in the respiratory tract to be absorbed into the blood stream and metabolized. NO has a half-life in the body of less than 6 s, and a radius of biological action of approximately 200 μm from its site of origin. Beyond this point it is inactivated through binding to sulthydryl groups of cellular thiols or by nitrosylation of the heme moieties of hemoglobin to form MetHg. MetHg reductase reduces NO to nitrates in the blood serum. In this study, the gNO was detected in the exhaled breath immediately after administering the NORS, and MetHg levels in the NO treated group were raised 5 and 30 min post treatment. Blood nitrite levels were also raised post treatment. A major metabolic pathway for NO is the conversion into nitrites and nitrates, collectively termed NOx, which exist as stable metabolite of nitric oxide within blood, tissue and urine. The rise in blood nitrite concentration in the NORS treated group is further proof, showing that inhaled NO was biologically available.

### Example 7

M. haemolytica bacterial cultures were isolated and obtained from the Agriculture and Agri-Food Canada Research Centre (Lethbridge, Canada). Bacteria were grown to 0.5 McFarland standard. 1 ml aliquots of these preparations containing approximately 2.5×10̂8 cfu/ml were stored at −80° C. On the day of the experiments the fresh stock was removed from the freezer, thawed, and 2 ml of Brain Heart Infusion (BHI) was added. Cultures were further diluted with BHI to achieve OD₆₀₀ of 0.1. Two different serotypes of M. haemolytica were used. These serotypes were originally isolated from bovine nasopharyngeal swabs, and subsequently confirmed by biochemical and PCR assays as M. haemolytica. They were serotyped in the laboratory, against reference sera, which was generated in rabbits.

NORS at different strengths was tested for efficacy against M. haemolytica serotypes. Saline was used as control. NORS (900 μl) was added to separate 1.5 ml sterile Eppendorf tubes. One hundred microliter of culture containing each serotype at 106 cfu/ml (OD₆₀₀ 0.1) was then added to each tube and incubated for 30 s, 1, 2, 5, and 10 min. Following incubation, samples from each tube were serially diluted and were plated on both BHI and blood agar sheep plates. Plates were incubated at 37° C. overnight (O/N). Each experiment was done in triplicate and repeated three times.

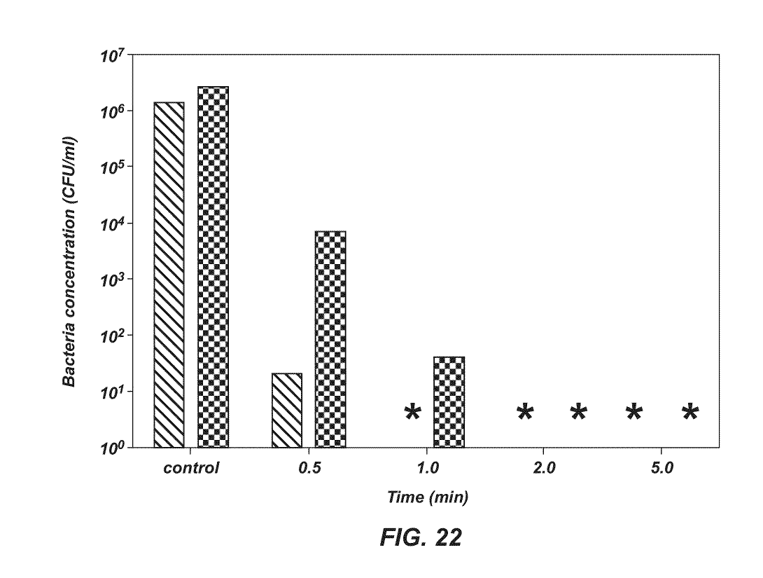

Using NORS, even for 0.5 min, resulted in significant (P<0.05) inhibition of M. haemolytica, compared to the control. Using NORS for 1 min caused a complete eradication of one serotype of this bacteria and 2 min for both serotypes (FIG. 22). Both serotypes that were used here are isolates from feedlot cattle.

Madin-Darby bovine kidney (MDBK) cells (ATCC CCL 22) were grown in Eagle's minimum essential medium (MEM) containing 10% fetal bovine serum. Infectious Bovine Rhinotracheitis (IBR), Bovine Respiratory Syncytial Virus (BRSV) and Bovine parainfluenza-3 (PI-3) were used throughout the experiments. These viruses were propagated in MDBK cells in Dulbecco's modified Eagle's medium (DMEM) supplemented with 2% fetal bovine serum and stored at −80° C. until use. The amount of virus was measured by a plaque assay on MDBK cells.

Virucidal activity was tested using equal volumes (0.025 ml) of virus suspension, containing 10̂3-10̂7 plaque-forming units (PFU/ml) of each of the 3 viruses and NORS. The two volumes were mixed together and incubated for 1 or 10 min at room temperature. The viruses were diluted with PBS containing 2% fetal bovine serum (FBS) and the number of infectious virus in each preparation was measured by a plaque assay.

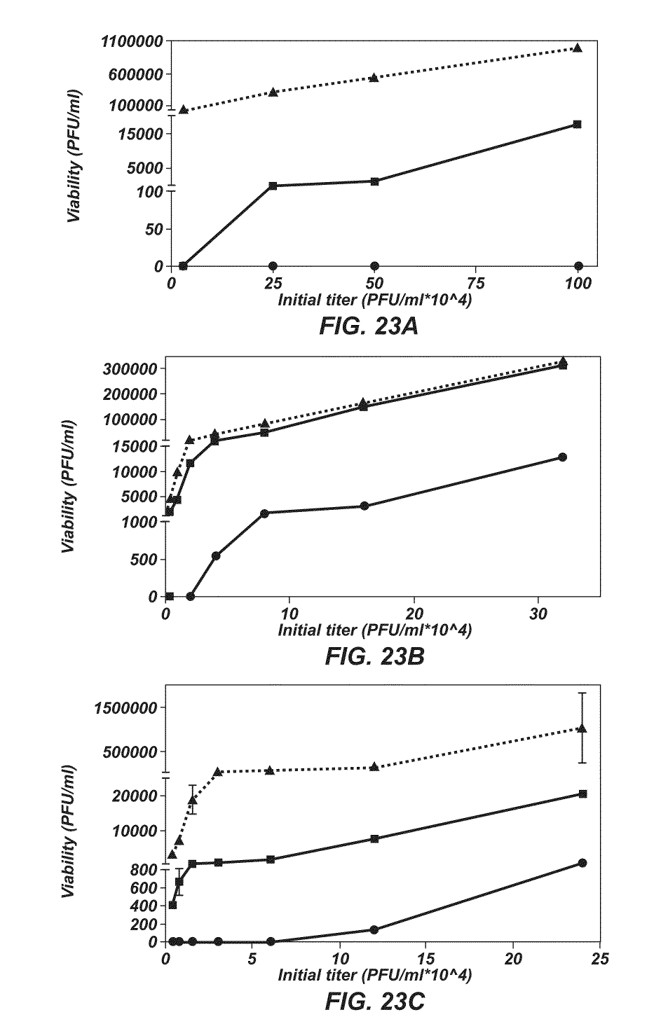

As can be seen in FIG. 23, IBR is the most susceptible virus to NORS, with a complete eradication with all initial titers after 10 min exposure and a significant (P<0.05) reduction for all titers after 1 min. PI3 was a bit less susceptible but still with significant reduction in viability at all titers, both after 1 and 10 min. The least susceptible was BRSV with no significant difference after 1 min exposure but a significant (P<0.05) reduction in viability following 10 min exposure at all titers. The ability of NORS to eradicate the virus was found to be in direct correlation with the initial titer.

Data in these exposure experiments were expressed as mean standard deviation (S.D.). Statistical analysis of data obtained in all experiments, were performed using a one-way analysis of variance (ANOVA) and Tukey's Multiple Comparison Test. A value of P<0.05 was considered statistically significant. Data analysis and graphical presentation were done using a commercial statistics package (Graphpad-Prism V 3.0, GraphPad Software Inc., USA).

These results indicate that NO releasing from NORS has an antimicrobial effect on two serotypes of M. haemolytica and three different viruses that are playing a major role in the BRDc. Additionally, these data show that NORS can eradicate (bacterial) or reduce (viral) these microbes within minutes. NO may act directly as an antimicrobial agent within the nasal mucosa and reduce bacterial/viral loads to a level that can be overcome by the body's immune system. Further, gaseous NO escaping from the surface, as confirmed in the exhaled gases in these animals, may have an antimicrobial effect throughout the entire respiratory tract.

The bactericidal mechanism of NO action is multifaceted. It is possible that NO produces a chemical modification of surface thiols or metal centers involved in critical enzymatic or regulatory function. Inactivation of cysteine proteases could be a general mechanism of NO related antibacterial activity. Further it is possible that when thiol levels are saturated, bacterial cell death occurs. Eukaryotic cells have much higher thiol levels and can cope with high levels of NO better than prokaryotes/microbes. Thus, host cells should tolerate NO resulting nitrosative stress more effectively than bacteria.

NORS results in the complete eradication of viral titers of 5.0×10̂4 PFU/ml, which are associated with viral infection. This virucidal activity occurs within 10 min for IBR and PI3 and 99% inhibition for BRSV. It has been suggested that NO inhibits viral proteins, RNA synthesis and viral replication by modifying molecules such as reductases and proteases required for replication. However, little is known about the antiviral mechanism by which NO acts. One of the plausible mechanisms of antimicrobial activity of NO involves the interaction of this free radical (and a reactive nitrogen intermediate) with reactive oxygen intermediates, such as hydrogen peroxide (H₂O₂) and superoxide (O₂) to form a variety of antimicrobial molecular species. Additionally, nitric oxide may be able to affect surface proteins, by nitrosylation of the cysteine moieties within its structure. This could alter or prevent the fusion of the virion with the epithelial cell membrane. Each virus has its line of surface glycoproteins to which NO could bind and disrupt the infection process. Enveloped viruses enter cells by fusing with the cell plasma membrane in a complex process of attachment and penetration. Virus entry requires the presence of complementary binding partners on the virus and on the host cell. NO may act as a neuraminidase inhibitor in Influenza virus. PI3 has both neuraminidase and haemoglutanin and thus its fusion may be inhibited through those being nitrosilated by NO. IBR has glycoproteins such as—gB, gC, gD, gE, gH, gK and gL that are required for virus entry. BRSV has two major membrane proteins, the fusion protein and the attachment glycoprotein, and also the small hydrophobic protein (SH) and M2 protein. Nitrosylation of these proteins by NO may reduce their ability to infect host cells.

Cytotoxicity caused to microbes does not necessarily have the same effect on host cells. Host cells are not immune to the high levels of NO released, however, unlike bacteria, they have evolved multiple defences against nitrosative injury. Eukaryotic cells have much higher thiol levels (glutathione) and can cope with higher levels of NO nitrosative stress better than prokaryotes/microbes. Thus, host cells should tolerate gNO stress more effectively than bacteria.

### Example 8

Commingled, multi-sourced, mixed-breed beef calves were purchased from auction markets for these studies. All studies were conducted at Cattleland Feedyards Ltd., a 25,000 head full service feedlot, located near Strathmore, Alberta, Canada, with all management practices adhering to the Canadian Council of Animal Care and Canadian Beef Cattle Code of Practice guidelines. In addition, the research protocols were reviewed and an Experimental Studies Certificate issued by the Veterinary Drug Directorate of Health Canada. Animals identified for the study were to be approximately 6 months old with an estimated initial mean body weight of between 400-600 lbs. All animals were transported via commercial cattle liners to the feedlot. These calves were chosen in order to provide study groups with an anticipated high incidence of BRDc ranging between 30-40%, which is representative of the beef industry in Canada for these “assembled” herds of cattle typically acquired at auction marts. After a 24-hour acclimatization period, the calves were screened, enrolled, randomized, processed, weighed, and temperature measured with blood samples taken by a veterinarian.

During the 35 day trial, all animals were provided with a total mixed ratio and permitted ad libitum feed consumption according to the standard operating procedure of the feedlot. The final finishing ration met or exceeded National Research Council guidelines for beef cattle. The animals also had free access to water and were provided a straw bedding area with a roof covering.

Animals were screened for enrolment criteria into the study. Animals with a fever of 104° F. and higher were excluded and were not enrolled. Following screening, calves were enrolled and randomized as they were processed in the catch chute.

During processing calves were weighed, labeled with numbered ear tags, received routine vaccination (Pyramid FP 5 with Presponse SQ—Boehringer Ingelheim, Vision 7 Somnus—Merck, Bimectin Pour—Vetoquinol) and then administered a randomized single daily treatment of either a non-antibiotic, NORS or tilmicosin (Micotil®), a macrolide antibiotic. The animals were consecutively grouped into 8 separate pens: 4 pens containing NORS treated animals, and 4 pens containing Micotil® treated animals an average of 135 animals per pen. Of the 1080 enrolled animals, 541 received NORS and 539 received Micotil®.

Animals were restrained in a conventional hydraulic cattle-handling catch chute and given either treatment: (NORS) or control (antibiotic—Micotil®), 24 hours post arrival to the feedlot. NORS was delivered with a spray device specifically designed for the study. This solution was previously tested and verified to release 160 ppm NO in a 3 L/min gas flow of medical air. Each animal received 1 spray (8 ml), alternating into each nostril, twice, and 1 muzzle spray (8 ml), for a total of 40 ml, before being released into the feedyard pen areas. The duration of treatment administration was less than 5 seconds. Animals in the control cohort received an injection of Micotil® of 2 mL/100 lb.

While contained in their receiving pens the calves were monitored daily, for 35 days, by trained pen keepers, whom were blinded as to the treatment interventions, for clinical signs of illness. Animals identified by the pen keepers as appearing to be sick were “pulled” and brought into a barn. The animal was then placed in a catch chute and assessed by a veterinarian, who was blinded to the study, and assigned a clinical score to each animal. Briefly, clinical scores were designed to identify BRDc and were based on the appearance of four criteria as follows:

Lung Sounds (crackles, wheezes, plural friction rubs)—0=normal, 1=Mild, 2=moderate and 3=severe.

Depression—0=normal, 1=avoidance, slight head droop, normal movement, 2=head down, slow movement, will not face up, ear droop and 3=all previous but more advanced and difficult to move to chute.

Rumen Fill—0=null, 1=mild decrease, 2=moderate decrease, 3=empty.

Nasal discharge—0=none or small amount. Serous, 1=mild serous or cloudy, 2=moderate amount, cloudy or mucopurulent, 3=severe—purulent.

Ocular discharges—0=none or small amount. Serous, 1=mild serous or cloudy, 2=moderate amount, cloudy or mucopurulent, 3=severe—purulent.

Temperature—core temperature (rectal) above 103.5° F.

Rectal temperatures for the calves were collected at the start of the study and at “pull” time.

BRDc was diagnosed as positive based on temperature greater than 103.5° F. and a clinical pulmonary score of 3 or greater.

All pulled animals were rescued with a florfenicol antibiotic (Resflor).

During the follow-up period (day 35 to day 60), relapses requiring antibiotic intervention were recorded as part of the normal feedlot routine. At day 150, all animals were assessed for average daily weight gain and recorded.

Blood was collected, in heparinized tubes, from all animals while in the catch chute during processing by a veterinarian. Blood was immediately placed in centrifuge on location at 5,000 RPM for 10 min and serum was collected and frozen (−5° C.). The same procedure was repeated for all “pulled” animals at the day of the pull, and for all pulled animals on the last day of the study (day 35). Samples were sent to Guelph University (Ontario, Canada) for haptoglobin measurements.

Animals that died during the first 35 days of the study, for any reason, were sent for pathology testing (done by a board certified, blinded, veterinary pathologist). The following sections were tested: Lungs (left anterior, left middle, left caudal, right anterior, right middle, right caudal), heart, nasal mucosa, pharynx, Retropharyngeal L.N. and trachea. Diagnosis for the cause of death was determined by the pathologist.

Immunohistochemical staining for Mycoplasma Bovis, BHV-1, BRSV, M. haemolytica and H. somni was conducted at Prairie Diagnostic Services, Saskatoon SK on an automated slide stainer. Using the Code-On stainer, binding of the primary antibody was detected using biotinylated horse anti-mouse or goat anti-rabbit immunoglobulins and an avidin-biotin immunoperoxidase complex reagent, with 3,3′-diaminobenzidine tetrahydrochloride (DAB) as the chromogen. Using the Benchmark stainer, binding of the primary antibody was detected using a streptavidin-biotin amplification system.

Deaths of animals from day 35 to 150 were recorded and the farm's pathologist identified the cause of death.

In order to evaluate the difference in BRDc positive rates between NORS and Micotil® treated animals, a non-inferiority analysis was conducted. Specifically, the null hypothesis that the BRDc positive rate in the Bovinex group (pB) is more than 7.5% higher than the rate in the Micotil® group (pM) was tested using a nonparametric bootstrap:
H0: pB−pM≧7.5%
   vs.
H1: pB−pM<7.5%

This non-inferiority test is one-sided, and compared to a 0.05 Type I error probability. All other hypothesis tests presented in this study are 2-sided and compared to a 0.05 Type I error probability. Point estimates and 95% confidence intervals for the percentage of positive animals in both groups, are also calculated using the bootstrap method, and account for the correlation among animals grouped together within the same pen.

In order to evaluate the effect of treatment on 35-day weight change, a linear mixed effects model was fit to the data. Specifically, the effect of treatment on weight change was evaluated while controlling for baseline weight (fixed effect) and pen (random effect). Confidence intervals are based on the linear mixed effects model and account for the possibility of correlation of animals grouped together within the same pen.

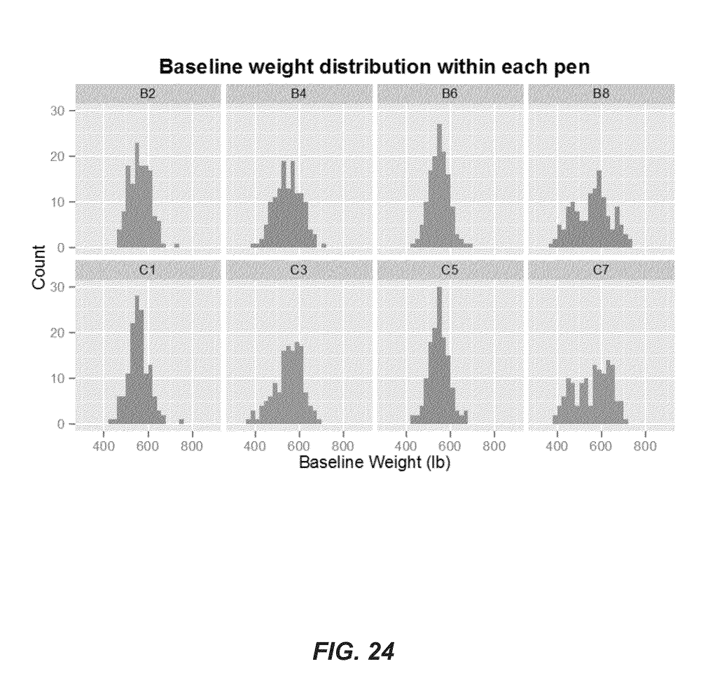

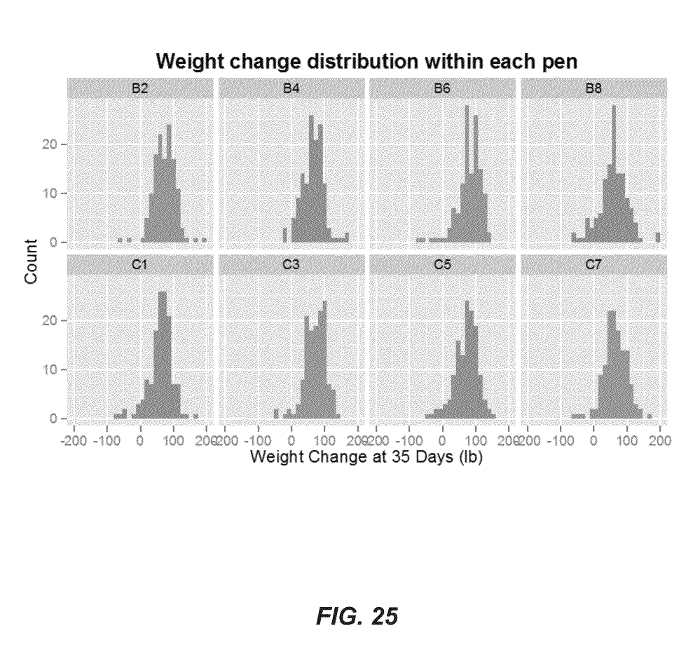

Initial weight on arrival and Weight gain appeared relatively consistent across pens. The average animal weighed in at 554 lb on arrival and gained approximately 68 lb. by Day 35 (FIGS. 24 and 25).

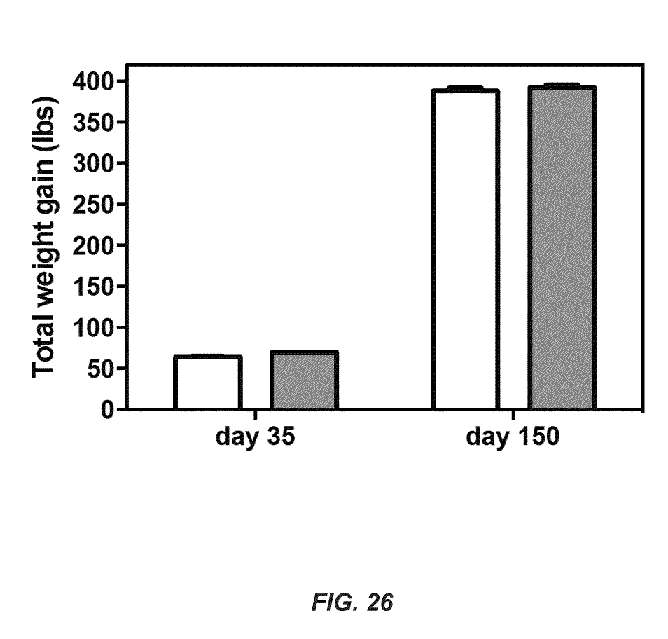

The average 35-day weight gain for the treated animals (NORS) with baseline weight of 554 lbs is 64.5 lbs, with an average daily gain of 1.84 lbs/day; and of a control (antibiotic) treated animal with baseline weight of 554 lbs is 70 lbs, with an average daily gain of 2.0 lbs/day (FIG. 26). Animals treated with antibiotic gained on average an additional 5.5 lbs over 35 days relative to those treated with NORS. Statistically, this difference in weight gain between these two cohorts is not significantly different than 0 (p>0.05).

When assessing the final weights at day 150 (FIG. 26), prior to the animals being sold and sent to slaughter, the average gain for NORS group is 388 lbs (2.59 lbs average daily gain) and 392 lbs for the antibiotic group (2.61 lbs average daily gain). There was no significant difference between the two groups (p>0.05).

The average daily weight gain (ADG) at day 35 and 150 were not significantly different between NORS and antibiotic treatment groups, both statistically and clinically. The health of the animal has been reported to directly affect the weight of the animal at finishing; and also the amount of days on feed required to attain the target weight. These are important economic metrics in the beef industry. While it is important that the health of the animal is protected, any alternative to sub therapeutic antibiotics must also preserve important economic cattle growth metrics such as the end weight, ADG and total days on feed. Although the study did not extend to finishing (180 days), the total days on feed to achieve the target weight gain would likely be the same for both cohorts.

Thirty-three animals were “pulled” from the antibiotic group for suspected illness, out of these 17 (1, 3, 5, 8 respectively per pen) were identified as BRDc positive. Sixty-three animals were “pulled” from the NO group for suspected illness, out of them, 28 (12, 0, 6, 10 per pen) were identified as BRDc positive. The prevalence of BRDc between the antibiotic and NORS did not differ significantly (p=0.3).

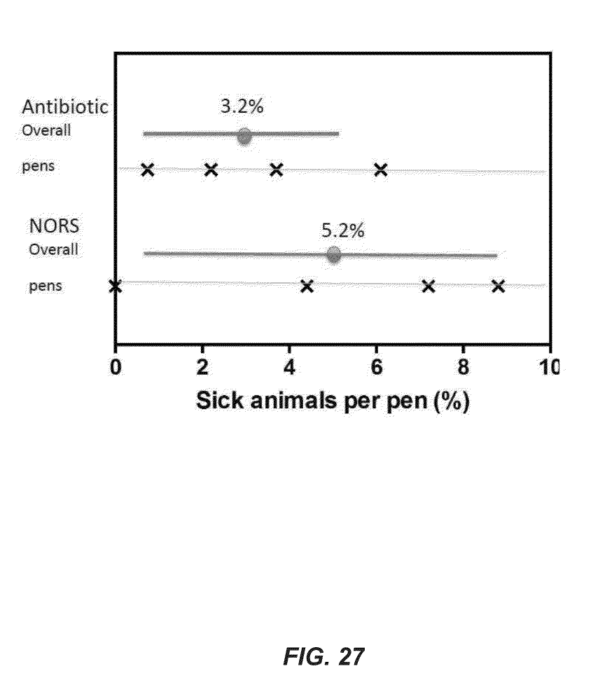

BRDc positive incidences varied substantially across the 8 pens (FIG. 27) with incidences ranging from 0% (0 infected among 136 animals in pen B4) to 9% (12 infected among 136 animals in pen C1). The average BRDc positive rates are 3.2% for the antibiotic group and 5.2% for NORS—Table 6 and FIG. 27. This difference was not found to be statistically different (P>0.05).

TABLE 6
BRDc positive incidences in each one of the 8 pens and a total for each cohort.

| | Control (antibiotic) | | | | | Treatment (NORS) | | | | |
|---|---|---|---|---|---|---|---|---|---|---|
| Pen | B2 | C3 | B6 | C7 | total | C1 | B4 | C5 | B8 | total |
| # of BRDc positive animals | 1 | 3 | 5 | 8 | 17 | 12 | 0 | 10 | 6 | 28 |
| Total number of animals | 135 | 135 | 135 | 132 | 537 | 136 | 136 | 137 | 134 | 543 |
| % of BRDc positive animals | 0.7 | 2.2 | 3.7 | 6.2 | 3.2 | 8.8 | 0 | 7.3 | 4.5 | 5.2 |

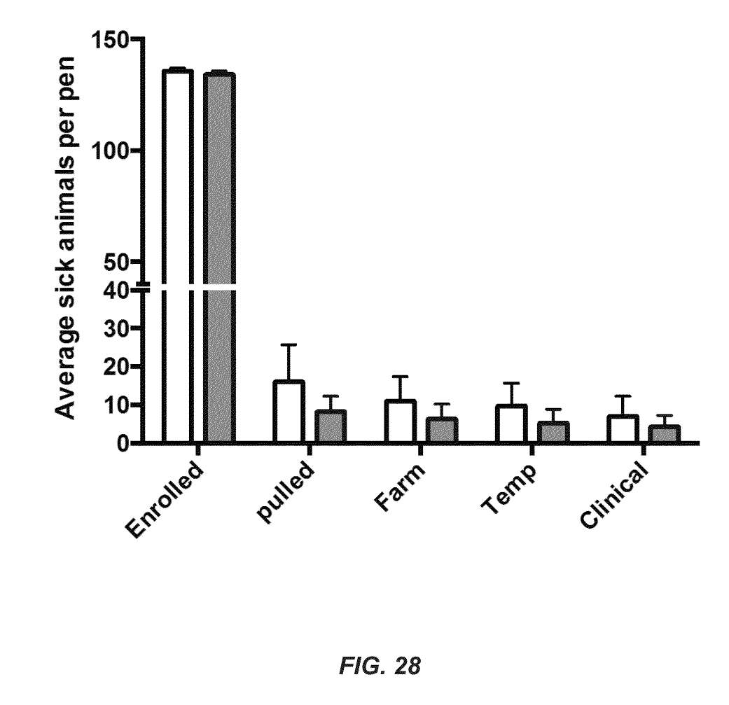

The definition of BRDc can range from a practical qualitative assessment to scientifically measured quantitative measurements. A quantitative definition of BRDc was used in this study, as described above. However, because this approach is still limited by the accuracy of the number of animals identified for general morbidity then “pulled” by the pen keepers as being “sick”, four definitions of BRDc are included as a comparison. The first definition of BRDc is the general morbidity pull rate (“pulled”) by the pen keepers (cowboy). These animals may include lame, bloated, and animals with an overall appearance of being “sick.” The second definitions of BRDc (“farm”) are the animals that were identified as BRDc positive as defined by the feedyard (taken from raw data score provided by the feedlot veterinarian). The third definition of BRDc (“temp”) is an undifferentiated fever based solely on the rectal temperature of 104° F. or higher at time of “pull.” The final definition of BRDc (“clinical”) is the study definition, which includes temp of 103.5° F. or higher and clinical score of 3 or greater. FIG. 28 compares these different diagnostic definitions of BRDc. There were no statistically significant differences between diagnostic definitions for BRDc (Table 7). In addition there was no statistically significant difference between NORS and antibiotic cohorts within each of the individual diagnostic definitions. These data validate that whether BRDc was qualitatively diagnosed by the pen keeper, or scientifically diagnosed using more quantitiative methods, the NORS was at least as effective in reducing incidence of BRDc as the antibiotic treatment.

TABLE 7
Statistical ANOVA analysis of the four different methods to assess
BRD.

| Turkey's multiple | | | |
|---|---|---|---|
| comparisons test | Mean Diff. | 95% CI of diff. | Significant? |
| pulled vs. farm | 3.500 | −29.12 to 36.12 | No |
| pulled vs. fever | 4.625 | −30.71 to 39.96 | No |
| pulled vs. clinical | 6.500 | −47.86 to 60.86 | No |
| farm vs. fever | 1.125 | −1.593 to 3.843 | No |
| farm vs. clinical | 3.000 | −18.74 to 24.74 | No |
| fever vs. clinical | 1.875 | −17.15 to 20.90 | No |

Following the initial pull and assessments all animals received a different antibiotic (Draxxin) treatment. As part of the normal commercial tracking in the feedyard, the animals were followed until day 150 for relapse and further antibiotic administration recorded. The number of relapses in the antibiotic and NORS cohorts were 10 and 11 respectively.

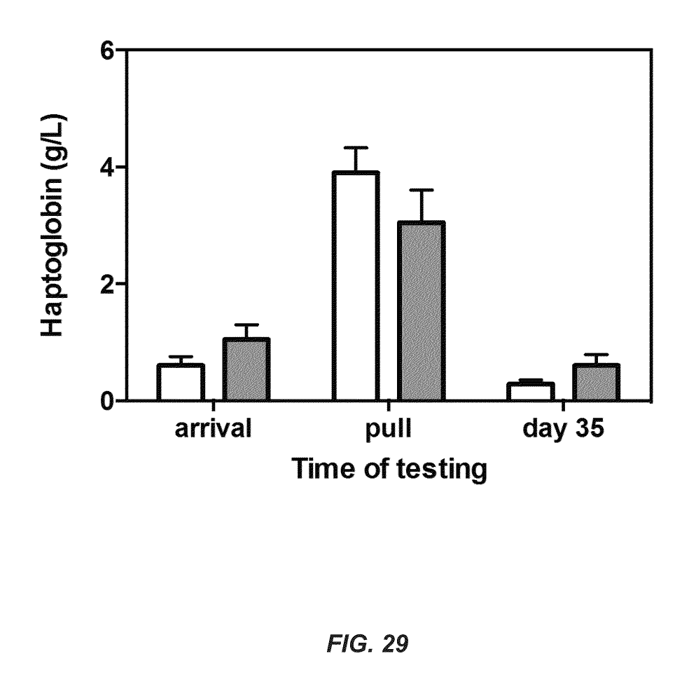

Blood samples for haptoglobin analysis were obtained from 1) healthy animal at processing identified retrospectively (controls), 2) sick animals identified as having BRDc at the time of pull, and 3) samples taken on day 35 from the same BRDc animals identified as sick. As can be seen in FIG. 29, there was a highly significant difference in mean haptoglobin levels between groups (ANOVA p<0.0001). End of study haptoglobin levels was not found to be significantly different from arrival levels. Both cohorts demonstrated a higher mean haptoglobin level on the day they were pulled, and diagnosed as having BRDc, as compared to the levels on day of arrival at the feedyard, and on day 35 when they were presumably healthy. On day of arrival the NORS group started with average value of 0.6 g/L, then had 3.9 g/L average on day of pull, then returned to 0.3 g/L at the end of the study (day 35). The healthy control group had a similar trend, starting with 1.05 g/L, rising to 3.05 g/L at pull time and then returning to 0.6 g/L on day 35. There was no significant difference between the control (antibiotic) and treatment group.

Further analysis revealed that animals that were identified initially as being sick and pulled when stratified as having BRDc, based on the study definition, had a mean value of 3.61±2.3 g/L whereas the animals that did not meet this definition had a haptoglobin level of 2.68±1.8 g/L. However, this difference was not statistically different. When looking at arrival haptoglobin values of 20 control animals that were not pulled at all during the study, the value (0.69±0.8 g/L) was statistically similar, though slightly lower, to arrival value for animals that got pulled during study (0.83±1.1 g/L).

Acute-phase proteins, such as Haptoglobin, are synthesized by the liver as a portion of the immune system's acute response to infection. Haptoglobin, in cattle, has been found to increase in response to multiple pathogens during natural or experimental exposure. Determining haptoglobin levels as a diagnostic tool appears to be promising in early detection and in assessing morbidity due to BRDc, specifically attributed to M. haemolytica infection. Results found in this study agree with others, showing increased value of haptoglobin at pull time. Pulled animals with higher clinical scores (3 and above) and temperatures of above 103.5° F. showed a higher haptoglobin level as compared to general “pulled” animals. No higher values of haptoglobin were observed upon arrival in the group of animals that got pulled later as compared to animals that never got pulled. This confirms that animals were healthy on arrival and enrollment.

TABLE 8
Immunohistochemical staining for 5 different bacteria and viruses
associated with BRDc.

| ANIMAL | Mycoplasma | | | | |
|---|---|---|---|---|---|
| # | bovis | BHV-1 | BRSV¹ | M. hemolytica | H. somni |
| 1024 | +++ | − | − | ++ | − |
| 1124 | − | − | + | ++++ | ++++ |
| 1305 | − | − | ++ | − | − |
| 1325 | − | − | ++ | + | − |
| 1353 | − | − | − | ++++ | ++++ |
| 1391 | − | − | − | ++++ | ++++ |
| 1487 | − | − | +++ | + | ++ |
| 1508 | − | − | ++ | − | − |
| 1518 | − | − | + | − | − |
| 2006 | − | − | − | ++++ | ++++ |
| 2023 | ++++ | − | ++ | + | ++ |
| 2152 | | − | ++ | ++++ | ++++ |
| 2279 | +++² | − | − | ++++ | ++++ |
| 2300 | − | − | ++ | − | − |
| 2315 | − | − | + | − | + |
| 2326 | ++++ | − | ++ | + | −³ |
| 2382 | − | − | + | ++ | − |
| 2501 | − | − | − | − | + |

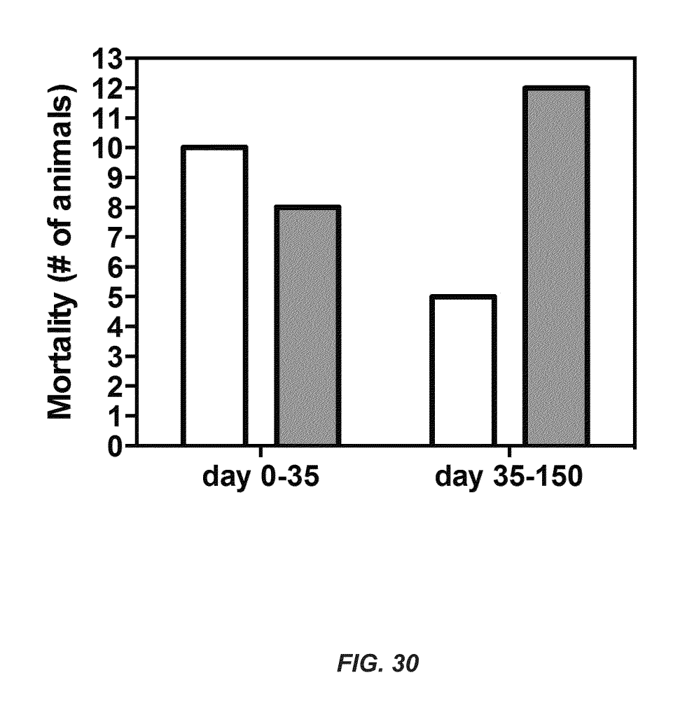

18 animals died during the 35 days of the study. Out of them, 8 were from the antibiotic group and 10 were from the NORS group (FIG. 30). Table 8 summarizes the immunoperoxidase results, an assay used to confirm the presence of infection, for the bacteria and viruses that were tested and Table 9 summarizes the pathologist diagnosis of cause of death.

TABLE 9
Cause of death (as defined by the pathologist) for animals that
died in the first 35 days

| ANIMAL # | DIAGNOSIS |
|---|---|
| G1024 | 1. Necrotizing myocarditis |
| | 2. Suppurative bronchopneumonia |
| G1124 | Fibrinous pneumonia |
| G1305 | 1. Necrotizing myocarditis |
| | 2. Fibrinous pneumonia |
| G1325 | Fibrinous pneumonia and septicemia |
| G1353 | Fibrinous pneumonia |
| G1391 | Fibrinous pneumonia |
| G1487 | 1. Necrotizing myocarditis |
| | 2. Fibrinous pneumonia |
| G1508 | 1. Necrotizing myocarditis |
| | 2. Fibrinous pneumonia |
| G1518 | Free gas bloat |
| G2006 | Fibrinous pneumonia |
| G2023 | Fibrinous pneumonia |
| G2152 | Fibrinous pneumonia |
| G2279 | Fibrinous pneumonia |
| G2300 | Necrotizing myocarditis |
| G2315 | 1. Free gas bloat |
| | 2. Bronchitis |
| G2326 | 1. Peritonitis |
| | 2. Bronchopneumonia |
| G2382 | 1. Necrotizing myocarditis |
| | 2. Fibrinous pneumonia |
| G2501 | Fibrinous pneumonia and pleuritis |

Immunohistochemical staining was conducted by a certified third party diagnostic laboratory (Prairie diagnostics, Saskatoon, Canada). As can be seen in Table 8, most mortality was correlated with high BRDc associated bacterial loads of M. haemolytica and H. somni. There was only one exception (2501) based on cause of death with “Fibrinous pneumonia and pleuritis” that was not associated with a high bacterial concentration. Animals (1518, 2315) that had very low to no microbes detected, and cause of death according to the pathologist report was from free gas. Four animals (2300, 1508, 2382 and 1305) died from necrotizing myocarditis, and thus did not have high concentration of microbes associated with BRDc.

When looking at the mortalities from day 35-150, 12 more animals died in the antibiotic group while only 5 died in the NORS treatment group (FIG. 29). However, only 3 deaths in the control and 1 in the treatment were diagnosed by the feedlot as BRDc related deaths.

This large controlled 1,080 calf study showed that NORS treatment was non-inferior, as compared to the common practice of metaphylactic antibiotic administration, in preventing BRDc in cattle arriving at a commercial feedlot. This study was statistically powered, with enough animals enrolled, to sufficiently evaluate cattle mortality and other secondary health and economic metrics. Mortality was similar for the two cohorts during the first 35 days. Interestingly, at day 150 there were significantly (almost 50%) more deaths in the antibiotic cohort. However, when histopathology and respiratory pathogens were factored into the cause of death attributed to respiratory disease, the difference in mortality between groups was not significant. It is unknown why overall mortality was lower in the nitrosylite cohort, but, without wishing to be bound by theory, it is believed that the NO not only had a direct effect on the microbes and innate immune system, but also had a positive effect on the acquired immune system, which may have lasting immunological benefits.

The exclusion criterion of febrile animals as used in this study is divergent from antibiotic studies where animals are enrolled only if they have high temperatures. Antibiotic studies are generally designed to test therapeutic effect rather than prophylactic effect (although in practice they are being used for prophylaxis). In contrast, this study was designed specifically to test the prophylactic effect of NORS to reduce BRDc. It was surprising, that NORS not only demonstrated equivalence to prophylactic antibiotic use, but also may have had a lower mortality rate than antibiotics. In addition there were no differences in relapses between the two cohort groups. Antibiotic effectiveness to “prevent” BRDc is linked to how long the antibiotics persist within the host; so loading doses are high enough to prevent the exacerbation of specific bacterial species. For example, it is claimed that tulathromycin remains in the pulmonary system for as long as 28 days in order to prevent BRDc. NO, on the other hand, has a short acting bioavailability and tissue residue (released from NORS) but also has a wide variety of immediate and long term beneficial characteristics that may help prevent BRDc. To name a few, NO is anti-viral, anti-bacterial, a mucolytic, a bronchodilator and a key. Without wishing to be bound by theory, these collective short term effects of NORS on the pulmonary microbiome, and the long term immunomodulatory effect triggered by the NO, may enable the host to resist the complex insults associated with the pathogenesis of BRDc.

BRDc is a medical challenge in veterinary medicine since clinical diagnosis can be difficult. The clinical diagnosis in scientific studies of BRDc is typically based on a combination of clinical signs including lethargy, anorexia, abnormal breathing patterns (all assessed by the pen keeper), and increased rectal temperature. However, in commercial feedyards, pen keepers routinely identify sick animals. In order to address this diagnostic spectrum, 4 different definitions of BRDc were compared, which showed that there were no significant differences among them. Importantly, the way BRDc was defined did not change the primary outcome-comparing incidence of BRDc between NORS and the tilmicosin metaphylactic treatments. From a practical perspective, the total number of pulled animals by pen keepers were close to double those clinically defined as having BRDc. However, the ratio of incidence of BRDc between the two cohorts remained the same.

A population of calves at high risk to develop BRDc was evaluated. The calves were weaned early, obtained from auction markets, commingled and had up to 24 hours of transport time to the feedlot. All of these factors are known to contribute to higher incidence of BRDc. In addition, the location of the study was in Western Canada which entailed harsh winter conditions. Daily temperatures during the study were as low as −32° F. and two severe snowstorms occurred. Despite these harsh environmental and predisposing conditions, it is unknown if the sample population represented calves at high-risk to BRDc. In order to approximate the incidence of BRDc in a negative control, data from published studies evaluating tilmicosin was analyzed. By extrapolating BRDc incidence in the negative controls from over 30 studies with over 18,000 animals, without considering how many animals were in each study, it was determined that the average incidence of BRDc in those combined studies was about 49±23%. As a conservative estimate, it was concluded that the incidence in a negative control in this study could have been 26% or higher. However, without a true negative control, it is impossible determine the actual BRDc risk level of the calves enrolled in this study.

Nitrosylites, such as NORS, could help protect the health of all animals including both natural antibiotic-free as well as animals placed in high-volume, high through-put feed yards. Antibiotics could then be saved for pathogen targeted therapy for the small portion of animals that do succumb bacterial respiratory infections.

### Example 9

Six 10-12 week-old ferrets were purchased from Triple F Farms and acclimated for 5-7 days prior to challenge. They were housed loose and together in a 12×18 ft. room for the duration of the study.

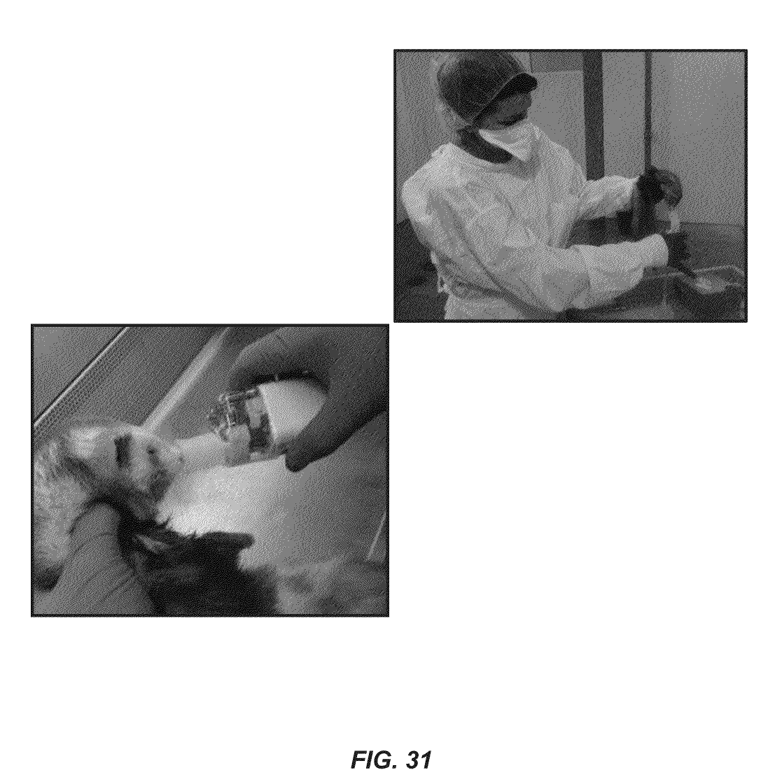

Six ferrets were stable for the study and were anesthetized with ketamine-xylazine and bled for nitrite baseline values. All animals were challenged with Influenza A/California/04/2009 virus by intranasal instillation of approximately 7.5×10⁴ pfu in 0.5 mL. Animals were then bled again to assess serum nitrite levels. Blood samples were obtained 30 and 240 minutes post treatment and analyzed for serum nitrites with a chemiluminescent analyzer. Within 5-10 minutes of inoculation treatment interventions were administered. One cohort of ferrets (n=3) received approximately 2 mL NORS over a ten minute period with a small volume nebulizer at 7 L/min. Another cohort of ferrets (n=3) received approximately 2 mL saline over a ten minute period with a small volume nebulizer at 7 L/min (FIG. 31).

The results were analyzed using the unpaired Student's t-test for comparison between any two groups. Group means were statistically tested by least squares means (two-tailed t-test). For experiments with multiple (more than 2) sets, Statistical analysis of data obtained were performed using a one-way analysis of variance (ANOVA) and Tukey's Multiple Comparison Test Data analysis and graphical presentation were done using a commercial statistics package (Graphpad-Prism V 3.0, GraphPad Software Inc., USA). Unless otherwise specified, p<0.05 indicated statistical significance. Results were reported as the mean±standard deviation.

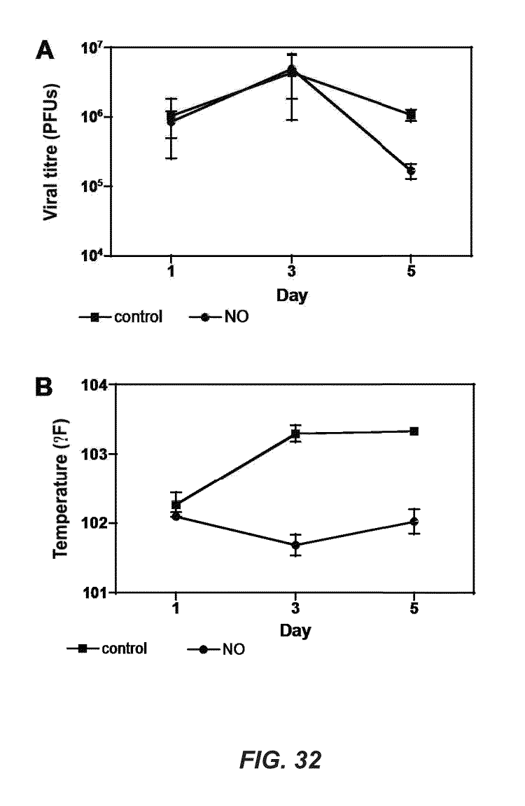

Nitrites in the serum were significantly elevated at 30 minutes (p<0.05) and returned to baseline after 240 minutes post NORS treatment as compared to baseline values (FIG. 32A). Moreover, when the NORS treatment was administered, there were significant levels of NO detectable by the AeroNOx™ (Pulmonox, Canada) device. These results show that the bioavailability of NO produced from NORS was demonstrated in the serum after treatment.

The average temperature for control versus treated animals, after 3 and 5 days was significantly (P<0.05) higher (FIG. 32B). This shows a systemic effect of the NORS treatment on the ferrets.

While the forgoing examples are illustrative of the specific embodiments in one or more particular applications, it will be apparent to those of ordinary skill in the art that numerous modifications in form, usage and details of implementation can be made without departing from the principles and concepts articulated herein. Accordingly, no limitation is intended except as by the claims set forth below.

What is claimed is:

1. A liquid nitric oxide releasing solution (NORS) for veterinary use, comprising at least one nitric oxide releasing compound and at least one acidifying agent, wherein the NORS provides an extended release of a therapeutically effective amount of nitric oxide gas (gNO).

2. The solution of claim 1, wherein the at least one nitric oxide releasing compound is selected from the group consisting of a nitrite, a salt thereof, or any combination thereof.

3. The solution of claim 2, wherein the nitric oxide releasing compound is sodium nitrite.

4. The solution of claim 1, wherein the amount of the at least one nitric oxide releasing compound is less than about 0.5% w/v.

5. The solution of claim 1, wherein the at least one acidifying agent is an acid.

6. The solution of claim 5, wherein the acid is citric acid.

7. The solution of claim 1, wherein the amount of the at least one acidifying agent is less than about 0.5% w/v.

8. The solution of claim 1, wherein the therapeutically effective concentration of gNO is about 160 ppm.

9. The solution of claim 1, wherein the NORS is a saline-based solution.

10. The solution of claim 1, wherein the gNO is released over a period of at least 30 minutes.

11. The solution of claim 1, wherein the gNO is released over a period of at least 4 hours.

12. The solution of claim 1, wherein the gNO is released over a period of at least 8 hours.

13. The solution of claim 1, wherein the gNO is released over a period of at least 12 hours.

14. The solution of claim 1, wherein the gNO is released over a period of at least 24 hours.

15. A liquid nitric oxide releasing solution (NORS) for veterinary use comprising at least one nitric oxide releasing compound and at least one acidifying agent, wherein either the at least one nitric oxide releasing compound or the at least one acidifying agent is present at a concentration of less than about 0.5% w/v, and wherein the NORS releases a therapeutically effective amount of nitric oxide gas (gNO).

16. The solution of claim 15, wherein the at least one nitric oxide releasing compound is selected from the group consisting of a nitrite, a salt thereof, or any combination thereof.

17. The solution of claim 16, wherein the nitric oxide releasing compound is sodium nitrite.

18. The solution of claim 15, wherein the at least one acidifying agent is an acid.

19. The solution of claim 18, wherein the acid is citric acid.

20. The solution of claim 15, wherein the concentration of the at least one acidifying agent is less than about 0.5% w/v.

21. The solution of claim 15, wherein the concentration of the at least one nitric oxide releasing compound is less than about 0.5% w/v.

22. The solution of claim 15, wherein the concentrations of both the at least one nitric oxide and the at least on acidifying agent are less than about 0.5% w/v.

23. The solution of claim 15, wherein the therapeutically effective concentration of gNO is about 160 ppm.

24. The solution of claim 15, wherein the NORS is a saline-based solution.

25. A method for the treatment of a disease or disorder in a veterinary subject comprising administering to the subject a liquid nitric oxide releasing solution (NORS) having at least one nitric oxide releasing compound and at least one acidifying agent, wherein either the at least one nitric oxide releasing compound or the at least one acidifying agent is present at a concentration of less than 0.5% w/v, and wherein the NORS releases a therapeutically effective amount of nitric oxide gas (gNO).

26. The method of claim 25, wherein the disease or disorder is a respiratory disease or disorder.

27. The method of claim 25, wherein the concentration of the at least one nitric oxide releasing compound is less than about 0.5% w/v.

28. The method of claim 25, wherein the concentration of the at least one acidifying agent is less than about 0.5% w/v.

29. The method of claim 25, wherein the concentrations of both the at least one nitric oxide releasing compound and the at least one acidifying agent are less than about 0.5% w/v.

30. The method of claim 25, wherein the NORS is administered as a spray.

31. The method of claim 25, wherein the NORS is administered intranasally to the veterinary subject.

32. The method of claim 31, wherein the veterinary subject is a bovine.

33. The method of claim 32, wherein the disease or disorder is Bovine Respiratory Disease Complex (BRDc).

34. The method of claim 25, wherein administration of the NORS at an administration situs provides a therapeutic effect at a situs distal from the administration situs.

35. A method of treating a respiratory condition in a veterinary subject, comprising administering a therapeutically effective amount of gaseous nitric oxide (gNO) to the subject from a nitric oxide releasing solution (NORS) deposited in the subject's nose, wherein occurrence of the respiratory condition is reduced.

36. The method of claim 35, wherein the subject is a bovine.

37. The method of claim 36, wherein the respiratory condition includes at least one of bovine respiratory disease (BRDc), bovine herpes virus 1 (BVH-1), bovine respiratory syncytial virus (BRSV).

38. The method of claim 35, wherein administration is metaphylactic.

39. The method of claim 35, wherein the administration is prophylactic.

40. The method of claim 35, wherein the NORS is dispensed into the subject's nostrils.

41. The method of claim 40, wherein the NORS enters the subject's nasal cavity.

42. The method of claim 40, wherein the NORS enters the subject's sinus cavity.

43. The method of claim 35, wherein the NORS has a nitrite content of from about 51 mM to about 100 mM.

44. The method of claim 43, wherein the amount of NORS deposited is from about 1 to about 100 ml.

45. The method of claim 43, wherein the amount of NORS deposited is from about 1 to about 50 ml.

46. The method of claim 43, wherein the amount of NORS deposited is about 32 ml.

47. The method of claim 35, wherein the NORS has an acid content of from about 5 mM to about 600 mM.

48. The method of claim 47, wherein the acid is citric acid.

49. The method of claim 48, wherein the acid content is from about 5 mM to about 50 mM.

50. The method of claim 47, wherein the acid is ascorbic acid.

51. The method of claim 50, wherein the acid content is from about 100 mM to about 600 mM.

52. The method of claim 36, wherein about 40% to about 100% of the NORS is deposited beyond the nasal vestibule.

53. The method of claim 36, wherein about 40% to about 80% of the NORS is deposited beyond the nasal vestibule.

54. The method of claim 36, wherein about 40% to about 60% of the NORS is deposited beyond the nasal vestibule.

55. The method of claim 35, wherein the therapeutically effective amount is from about 40 to about 400 ppm gNO.

56. The method of claim 35, wherein the therapeutically effective amount is from about 100 to about 220 ppm gNO.

57. The method of claim 35, wherein the therapeutically effective amount is about 160 ppm gNO.

58. The method of claim 35, wherein the occurrence of a respiratory condition is reduced by greater than about 40%.

59. The method of claim 35, wherein the occurrence of a respiratory condition is delayed by at least 7 days.

60. The method of claim 35, wherein the occurrence of a respiratory condition is delayed by at least 10 days.

61. The method of claim 35, wherein the occurrence of a respiratory condition is delayed by at least 14 days.

62. The method of claim 35, wherein blood MetHg levels of the subject increase upon administration.

63. The method of claim 62, wherein blood MetHg levels return to about pre-treatment levels within about 60 minutes of administration.

64. The method of claim 62, wherein blood MetHg levels return to about pre-treatment levels within about 30 minutes of administration.

65. The method of claim 35, wherein blood nitrites of the subject increase upon administration.

66. The method of claim 65, wherein blood nitrites return to about pre-treatment levels within about 60 minutes of administration.

67. The method of claim 65, wherein blood nitrites return to about pre-treatment levels within about 30 minutes of administration.

68. The method of claim 35, wherein a fraction of exhaled nitric oxide (F_{E}NO) is increased upon administration.

69. The method of claim 35, wherein gNO or NORS do not substantially affect blood cortisol levels in the subject.

70. The method of claim 35, wherein the veterinary subject is a commercially-salable animal.

71. The method of claim 70, wherein nitrite levels in meat obtained from the commercially-salable animal are substantially unchanged post-treatment.

72. The method of claim 35, wherein gNO is a microbicidal agent.

73. The method of claim 72, wherein gNO is a bactericidal agent.

74. The method of claim 72, wherein gNO is a virucidal agent.

75. A method of treating bovine respiratory disease in a veterinary subject, comprising administering a therapeutically effective amount of gaseous nitric oxide (gNO) to the subject from a nitric oxide releasing solution (NORS), wherein at least one treatment outcome after treatment with gNO is equivalent to the treatment outcome found after treatment with tilmicosin.

76. The method of claim 75, wherein a dosage of tilmicosin is from about 10 mg to about 20 mg per kg of subject body weight.

77. The method of claim 76, wherein the dosage of tilmicosin is from about 11 mg to about 15 mg per kg of subject body weight.

78. The method of claim 76, wherein the dosage of tilmicosin is from about 12 mg to about 14 mg per kg of subject body weight.

79. The method of claim 75, wherein the treatment outcome includes at least one of subject weight gain, morbidity, mortality, or a combination thereof.

80. The method of claim 79, wherein the treatment outcome includes subject weight gain.

81. The method of claim 79, wherein the treatment outcome includes subject morbidity.

82. The method of claim 79, wherein the treatment outcome includes subject mortality.

83. A method of treating a respiratory condition in a veterinary subject, comprising administering a therapeutically effective amount of gaseous nitric oxide (gNO) to the subject from a nitric oxide releasing solution (NORS) administered to the subject's nose, wherein at least one indicator of bioavailability of the administered gNO in the subject is increased.

84. The method of claim 83, wherein the NORS is deposited in the nose of the subject.

85. The method of claim 83, wherein the NORS is deposited on the nose of the subject.

86. The method of claim 83, wherein the NORS is deposited both on and in the nose of the subject.

87. The method of claim 83, wherein the at least one indicator of bioavailability of gNO includes at least one of increased MetHg levels, increased blood nitrate levels, increased fraction of exhaled gNO, or combinations thereof as compared to levels present in the subject prior to NORS administration.

88. The method of claim 87, wherein the at least one indicator of bioavailability of gNO includes increased MetHg levels.

89. The method of claim 87, wherein the at least one indicator of bioavailability of gNO includes increased blood nitrate levels.

90. The method of claim 87, wherein the at least one indicator of bioavailability of gNO includes increased FENO.

91. The method of claim 83, wherein the at least one indicator of bioavailability of gNO returns to about baseline levels within about 90 minutes of administration of the NORS.

92. The method of claim 83, wherein the at least one indicator of bioavailability of gNO returns to about baseline levels within about 60 minutes of administration of the NORS.

93. The method of claim 83, wherein the at least one indicator of bioavailability of gNO returns to about baseline levels within about 30 minutes of administration of the NORS.

94. The method of claim 83, wherein the respiratory condition includes inflammation of the upper respiratory tract.

95. The method of claim 83, wherein the respiratory condition includes a microbial infection.

96. The method of claim 95, wherein the microbial infection includes at least one of a viral infection and a bacterial infection.

97. The method of claim 96, wherein the microbial infection includes a viral infection.

98. The method of claim 96, wherein the microbial infection includes a bacterial infection.

99. A method of administering a nitric oxide releasing solution (NORS) to a veterinary subject comprising:
providing a nitric oxide releasing agent;
providing an acidifying agent;
combining the nitric oxide releasing agent and the acidifying agent to provide an activated NORS up to 24 hours before administration; and
administering the activated NORS to the subject.

100. The method of claim 99, wherein the NORS is activated up to 8 hours before administration.

101. The method of claim 99, wherein the NORS is activated up to 1 hour before administration.

102. The method of claim 99, wherein the NORS is activated up to 30 minutes before administration.

103. The method of claim 99, wherein the NORS is activated up to 10 minutes before administration.

104. The method of claim 99, wherein the NORS is activated up to 5 minutes before administration.

105. The method of claim 99, wherein the NORS is activated up to 1 minute before administration.

106. The method of claim 99, wherein the NORS is activated during administration.

107. A nitric oxide releasing solution (NORS) for veterinary use, comprising at least one nitric oxide releasing agent and at least one acidifying agent, wherein the NORS provides a therapeutically effective amount of gaseous nitric oxide (gNO), and wherein the NORS has an antimicrobial effect on Mannheimia haemolytica.

108. The solution of claim 107, wherein the NORS has an antimicrobial effect on at least one of Infectious Bovine Rhinotracheitis (IBR), Bovine Respiratory Syncytial Virus (BRSV), and Bovine Parainfluenza-3 (PI-3).

109. The solution of claim 107, wherein the nitric oxide releasing agent is present at a concentration of from about 51 mM to about 100 mM.

110. The solution of claim 107, wherein the nitric oxide releasing agent is present at a concentration of less than 0.5% w/v.

111. The solution of claim 107, wherein the therapeutically effective amount of gNO is from about 100 ppm to about 220 ppm.

112. The solution of claim 107, wherein the therapeutically effective amount of gNO is about 160 ppm.

113. The solution of claim 107, wherein the acidifying agent is present at a concentration of from about 5 mM to about 600 mM.

114. The solution of claim 107, wherein the acidifying agent is present at a concentration of less than 0.5% w/v.

115. A nitric oxide releasing solution (NORS) for veterinary use, comprising at least one nitric oxide releasing compound and at least one acidifying agent, wherein the NORS releases a therapeutically effective amount of gaseous nitric oxide (gNO) that is less than 160 ppm for an extended period.

116. The solution of claim 115, wherein the therapeutically effective amount of gNO is from about 40 ppm to about 155 ppm.

117. The solution of claim 115, wherein the therapeutically effective amount of gNO is from about 60 ppm to about 120 ppm.

118. The solution of claim 115, wherein the therapeutically effective amount of gNO is from about 80 ppm to about 100 ppm.

119. The solution of claim 115, wherein the therapeutically effective amount of gNO is released over a period of at least 30 minutes.

120. The solution of claim 115, wherein the therapeutically effective amount of gNO is released over a period of at least 4 hours.

121. The solution of claim 115, wherein the therapeutically effective amount of gNO is released over a period of at least 8 hours.

122. The solution of claim 115, wherein the therapeutically effective amount of gNO is released over a period of at least 12 hours.

123. The solution of claim 115, wherein the therapeutically effective amount of gNO is released over a period of at least 24 hours.

124. A nitric oxide releasing solution (NORS) for veterinary use comprising at least one nitric oxide releasing compound and at least one acidifying agent, wherein the NORS releases a therapeutically effective amount of gaseous nitric oxide (gNO) for longer than 30 minutes.

125. The solution of claim 124, wherein the therapeutically effective amount of gNO is from about 40 ppm to about 400 ppm.

126. The solution of claim 124, wherein the therapeutically effective amount of gNO is from about 100 ppm to about 220 ppm.

127. The solution of claim 124, wherein the therapeutically effective amount of gNO is about 160 ppm.

128. Use of a nitric oxide releasing solution (NORS) for the manufacture of a veterinary medicament for treatment of a respiratory disease or disorder in a subject, wherein the NORS releases a therapeutically effective amount of gaseous nitric oxide (gNO), and wherein at least one treatment outcome resulting from treatment with the NORS is equivalent to the treatment outcome resulting from treatment with tilmicosin.

129. The use of claim 128, wherein a dosage of tilmicosin is from about 10 mg to about 20 mg per kg of subject body weight.

130. The use of claim 128, wherein the dosage of tilmicosin is from about 11 mg to about 15 mg per kg of subject body weight.

131. The use of claim 128, wherein the dosage of tilmicosin is from about 12 mg to about 14 mg per kg of subject body weight.

132. The use of claim 128, wherein the treatment outcome includes at least one of subject weight gain, morbidity, mortality, or a combination thereof.

133. The use of claim 132, wherein the treatment outcome includes subject weight gain.

134. The use of claim 132, wherein the treatment outcome includes subject morbidity.

135. The use of claim 132, wherein the treatment outcome includes subject mortality.

136. Use of a nitric oxide releasing solution (NORS) for the manufacture of a veterinary medicament for treatment of a respiratory disease or disorder, wherein the NORS releases a therapeutically effective amount of gaseous nitric oxide (gNO), and wherein the NORS is deposited simultaneously in both nostrils of a subject.

137. The use of claim 136, wherein the NORS has a nitrite content of from about 51 mM to about 100 mM.

138. The use of claim 137, wherein the amount of NORS deposited in both nostrils is from about 1 to about 100 ml.

139. The use of claim 137, wherein the amount of NORS deposited in both nostrils is from about 1 to about 50 ml.

140. The use of claim 137, wherein the amount of NORS deposited in both nostrils is about 32 ml.

141. The use of claim 136, wherein the therapeutically effective amount of gNO is from about 40 ppm to about 400 ppm.

142. The use of claim 136, wherein the therapeutically effective amount of gNO is from about 100 ppm to about 220 ppm.

143. The use of claim 136, wherein the therapeutically effective amount of gNO is about 160 ppm.

144. Use of a nitric oxide releasing solution (NORS) in the manufacture of a veterinary medicament for the treatment of a disease or disorder in a subject, wherein a nitric oxide releasing agent and an acidifying agent are combined to provide an activated NORS up to 24 hours prior to administration, and wherein the activated NORS is administered to the subject.

145. The use of claim 144, wherein the NORS is activated up to 8 hours prior to administration.

146. The use of claim 144, wherein the NORS is activated up to 1 hour prior to administration.

147. The use of claim 144, wherein the NORS is activated up to 30 minutes prior to administration.

148. The use of claim 144, wherein the NORS is activated up to 10 minutes prior to administration.

149. The use of claim 144, wherein the NORS is activated up to 5 minutes prior to administration.

150. The use of claim 144, wherein the NORS is activated up to 1 minute prior to administration.

151. The use of claim 144, wherein the NORS is activated during administration.
